SEQUENCE LISTING (106 sequences)

SEQ ID NO: 1 - DNA, 42808 bp, Bacteriophage phi eiAU
catcggtacacgaagccgatcaggttctgcggatggtatctgacgatattacaaccttacgcgatgtgcgccgcgacgagtggcgcaatgcccgtcgcgcgggcacaagccgcgatcgcgaattcattaaatgggatgagaatgtgtcgctagttttccaaaaactttgcgatttgcgcgatagcgcacagtttttcatcgacacccatacgccaccggctattgccgagctttatttccggagaatttaccgtgattagaaaagtgcgccttaaaaaacgcatcctgaaaatgtgccgctgctgcggcgtggaaaagccgttgtacgaattccacaaatacaccggcaccacctgccggtcgccagacggacaccgggcgatctgcaaggtgtgtcgcaatgaacaggcccgcgagtatgcgcgccgtaaacgtgcaaagaatggagaataaaaccatggccactattaccaaaaaacaacgcgcagaacttcgcatgaaatttggtggccgctgtgcttattgcgggtgcgaactttcagatagggggtggcacgccgatcatgtagaaccggcattgcgtaagtgggagttcgttaaaaataaaacaagtggagtgctacaaactgcttctacgggggaattttggcgacctgaaaatgatacgctcgaaaacctgttcccatcctgtgctccatgcaatctatttaaggcaacttttagtgtagagatgtttcgagaacagatcgcagaacaggtaaaacgcgcacggtcacgcagcgtaaatttccgcacggcggagcgattcgggcttattaaggttattgatatgccggttgttttctggtttgagcggtatcaggaaggagcagatcaccaaggcgatagtagaaaagctagccgtaattgggaaaggtactcatgatgaatcacaaattattgcgccatcttggctacggtgaattcccggacgcggtcatcgatgccgaactgtgccgagtgatggccgcgaagtacaaaaactcaatccccggtgctctgcgccatttcgcccgagcgcgagccgcaacagtgcgcaatccgtcgctaaaatcggcactggtcaagatgggtgcgagtatctacccggaaaccgggatcgccaccctgcgcgcttgcctggacaagatgcacgccgctgcggtgcgtgaactgcgcgcgcaaggcattacgcccgatgaatatatccgggccgcgggggagcaacatggcacagtttaagcgccgcccgtaccagaaagcgatcacgggccacatcatcgcgcatgctcggtgcaacgtgtgggctacgatgggcagcgggaaaacgggcgcgacgatgtgggcgctagatgccatgtttagcaccggcattctagatgagtcggatcgcgttctgatcctcgccccgttgcgcgttgcgtctggcacttggccggaggaacagcgcaagtggaaatttcccgcgctgcgggttatcgatgccaccggtaacgccgagcaccggatcgaggcactggcaacatcggcgaatgtggtatgcctgaattacgacgtgctggaatggctggtcgagtattacggcaacgattggccgtttactgtcgtagttgccgatgaaagcacgcggttaaaatcgtatcgtagccgcggcggtagcaagcgggcccgcgcattggcgaaagtggcgcataagaaaatccgcaggtttatcaatctgaccggtacgccagcgcggaacggcttaaaggacgtgtgggggcagatgtggtttctcgatgcgggcgagcgccttggcaccagttatcaatcattctcagatcgctggttcgtcagtaagcaagtcggctcgtcaccacttgcgcgccagatatcgccacgcaccggggcggaaaccgagatccaccagaagtgcgcggacctcagcatcacgatcgacgcggcggagtatttcgggtgtgataagccggtagtcgtaccgatcgtagtcgagttgccgaagaaagcgcgcaagatctacgacgatatggaaaacgcgcttttcgctgaattggaaagcggcgaaatcgaggcctcgaacgcggcggcaaaaacggccaagtgtttacagatcgcgggcggcgcctgttacatcacgaccgacgatggcgaggcatccaaagagtggacggaaatccacaaggccaagctcgacgcgctggaatccatcatcgaggagctaaacggcagcccgttgctagtcgcgtaccagtataaacacgacctggtgcgcctgctaaaacgcttcccgcagggccgcgcgatgcgcaaggggttaaagggcaacaatgacatggccgattggaacgccggcaaggtgccgatcatgttcgtgcatccagccagcgcgggccatggcctgaacttgcaggacggcgggtgccatctggcctttttcaacgatacgtggaactatgagcaatatgcgcagatcgtcgagcgtatcggccccgtccgccagcaccaagccgggcacccgcgcacggtatacatatacatcattcaggcacgcggaacacttgatgaggttgtcgccctgcggcgcgacgacaaggccgaagtgcaagacctgttaatggactatatgaaacgcaaaaagaggagtaaatgatgacccgcatgctacggtctaatcccgtcgttgccgttcctgtaggcggtggcccggccatctacataccatgcccaaaggccattatgcggcgcggctttttgccagctggcgttagccaggtattgcagggccataaaaaatcgcaccgcgggtatgtgttccgccgggctaccaatcgtgagatcgcggcgttcgattgcgatatcggctatctcgcgccgtcagagttcagccactgagctattggcctcactgctgacgtaccacccgcgcaccggtgagatccgcgataaacgtaccggaaagcgcaagggggcctctaccccctccggtggggtaacagtcatcgtgaacgataaaacgatgtgggggccgcgtgtggcatgggtattacacactcggcaacctgtgccggatggcctgacggtgcgctgcatcgacggagggatcggacattatgcacagcgttggaccaatctggaattatgcaaacaggaagatattcgccttgacgaaagcgcgatagacggctacagttaattcgatttaacaaccggtgccgaatatatgactgcctattacaatgaaattgacccctacgccgcgcaatggctgcgtaatcttatcgcagaagggcatatcgcccctggtattgttgacgaacgatcgatcgaggatataacaccaaatgaactcaccgaatttacccagtgccacttcttcgccggaattggagtatggtcgctcgccctgcgccgcgcaggatggccggatgatcgaccggtctggaccggatcttgcccttgccagcctttcagcgcggcaggcaaaggcgcaggggttgctgacgagcggcacctgtggccggcattcttccatctcatcagccagtgcagccctagcgtcgtctttggtgagcaggtttcaagcaaggacggcctcggttggctcgacattgtacaaactgacttggaaaacgcgggatacgccagcgcagcggcagatttatgcgctgcgggcgtcggtgcgccgcacatccgacagcgattgtactgggtggccgacgccaaccaccagcgacaggaagggaagcagccccgccaccatgcggaaggatgggaaggacaggactttctgccgtctcgactatgcgacggagcaggggttagtagtgtctccgccgagagcggacgggtcgcaacgattcgatcaattaccgagacagggcaatctctgcgggtggccgacgccgagggcggtagatggggagaaaaactccagaacattggcgggggcactacgggagttggagcgcgggaaattatcttgcctgccggggtggtcacaaatgacgctgccgggccgactaacggccactggcgagattctgactggctcagctgccgggatggaaagtggcggccacttgaaccctggcacattcccgttggctaatgggattacctcccgagtggggacgattgcgcgcctacgggaactccatctgtgcgcagctcgcagaagaattcatccgggcatattctcgacacggagaaaaattaaaatgtctggctatcacgattcaaaaaccggcacccagaagattagaaaaattaaaatgtctggctatcccgattcaaaaacggcccccgaagataaagattgctggcgtacccccccggaggttttcagggtatgctgttcgtacatggggttctttcgaaatagacgccgcagcggcagatcacaatccccttgttgccgattactggacgctagcagataacgcgctggtgcaggattggagcggaaaacctgtatggtgtaatcccccgtatagcgacatcggcccctgggtagagaaagccgctacggcggaattttgcgtaatgctagttcccgctgacacgtcggttaagtggttcgcccccgcgggagaactcggggcgtccgttatttttatcacgcgtggccgtttgcggtttatccataacgcaacgggaaagccggggccgagtaacaaaatggggtcttgctttctggtctttggcggtagtcgacccggacgggtagattttgtaacgcgggctggcgtttatcaaatcggcgccccccgcaaagtgacggttaaaaggcgcgtccgtgcgccgcccaatgcaacataattttaacccaataggccgctgcgtctaccattaaaaaaaaaaatggttgcaaagttggcggcctacgcctatagttaaatcaacttaacaaagaaaagaggaatcgcagaaaatgaacgacgaaactcaataaaattgatagatctgattaaaggcccgagacgatatcgcatgcaaaacttagcgcactacataagcgagattctgtagccttacggtagaaacttaaaagccgagaatagggcgaattgttgagggtgcctgcgctttgggtgggtattcggtggaggaatatattaacagtgacggtatgagggccttataacgatgaaacgaatcaccgcaatcgcaatcataaccgccgctatcatcggcagttcgtatgtcggcactgtaggcgccgaggacatgaccgcgcatgataagtgcgaatacctggcgtataacggcccgtcggcaccagctagtgcagacgaccgcgacacggcaacgcttctatgccttaacgccgtaacagttgccgaagaaaaccccggcgtatcggttgacgtcctccgcggcattctcagcttgcaaggtgcgatgcagcacaacccggaaaaagaagccgatcaccgctggcgttcgctagccatcctgcacggtttcaacatccaacgcggcaattacaatacgggcggtgcaaaatgacctacttccttgccatgatcgcaatcatgctgacggcagtcactatcggcactatcgacaagaaagagaaagggctgtcggggctatctcgcgggttgctgcgtgtgctagcgatggccgcaatgtgccttagtttcttcgtggcgttcgatatggtggattttaagagcccgtattacgggtacgtaaaggatcagcacaagttaacgacagcgcttgtttttggcctaggtgccatcacgctatcgatcatatcgacgttcggaaagcgtaaataaccaaggggccgctaggcccctttttcggcttcatgcattcccattatcgcaagtgccttaaccgcaatccgctgtaggcgctccatctgtgccgtgttgagcttcccggtagaggccaccgccataatacccgataaactccctagcgccttaaaatcggccatggcggattgtatgatagccgtctgcgggtagccctcgtcgagtatggcctgcgcccgttccgccagattatcggcgacagctttaaccccgtcactcggcgtcatcttcgcttacctcgcctttttcttttcccttttgcggtaccgctgccggttgcggagggaaagggctgatcggctcgcgctgttcttcctgcgcggctggcggtgtgatatctaaaaattgtttgatgaatgacccagcgaaatcgatatagtcgcaagtgtagacgtgcatgcggtcttgatcagcgggcgtcaacatgttggggctcatcatgatcttgtgttggtggaaaccgacatagaggacgttaatcggtgctttgatcgatccgggcggcggaacgataacgcccaaaatccaacaataatgcacctggccatttccgaggctgatcggttcgcaaccgacgttcaaccactgcacctccgagtgcgggaaagttgcagcgatatctgttgcgtccttccgcaaatgccaatctacgccggctatgcggatcacttttttaggcgtcatcccgttgaactgagtcacgcccacgccttggctttggtattggtttgtcattgtgtcggctccaccgtgtttttacatcttgtgcattttttctagtgcggcaatacacgccgcggctagatcaacgagttcgtgctcgataccttcgcgcgacccttgctctttcttggtcatcagctcatggtattccatttctacaacggccatcataccgcccggctgggaaatatgacttgcccaagttgcaggatgttcatgaactcgttcccgtacactctccataaccgcactatgcgaatcatggtgtttcggatgacgataaccagtgacatcttccataacctccgtgccgccggatagcggatgatagtaatgatgatgttctgacaccggcgcgccatggtggcggcgcatccggcgatacattaatactcgcatttttacacctcgctaatcgcccggattgctccgggcgcgggttagacaggcttacgcggatttggtgccgagatgtgccaaaatcgtgttgatgatctggttagtctgggccgcctggctgttttgcaggaagatttccccggacagcttggcgttctgcgctttctggtcgcacaactgagtttgcagatcggtgatgaagttctgctggatcagttggcgagtggcggcgccctccgccgcgatggtcttctgcgtctcgcagcaacagcgggccatatcggcgctgacaccggcgaaaccgctggccacggcaaagcgggtatccgcaccgttgcggtcaacggtggcattaacccctgcgaagccctggcacaatgcggactggatgcccgcctgcccctggaagttcgcatactgagccgcagacagcccattagcgatcgtcatgttggtaccgttctggccttgcagagtttgcagggtgcccgcattcactgcggacgcgacggagttgataccatcgagcacggcggtagtcgcgatagccgcttcaccggcaacgccaccacggttaccccagccgccgccaaagccattgccgaaccaagagccgatcaggccaccaaccgcaccgccaagacctgcggcaccggcttcgccacccatcccgccggcggggatcagagtcatatcagacatgttaacacctctttgtgtttgttgaatttttgtaacaactaagttacacaaagagaataccccgcagatcggcgcttgccaatcacgcggggtggtatttttattaaattattgataactattgtattatactcggttcgataccaacaccctataccggcggggtggtgggccattgaatatcgggataactagccggattaatctcccgcaattttttaatatagcccatccattctaccaatttcgccttatcgccatcgcttatgatacctagtgatagctccgtttgccaatcgttcgtggtcttgcgggcctcttccaatagcgtattttttttatccagggcctgagttattaactcttcttgggttggcggtgggttagcaagtgccatcgcctccgtttcagaaataggcattaacccttctttaatgtaatgatcttgggacccatcggcgagataggcgtacaccatatttttagaatctttgaaatatttcatcgcatttcaacccagttaactagcgttcctgtgggacatgatacagaataagttgctccttgaggtattattgcgtaaagataatgccattgtgttagccatgatacgttgggggtattaccgccggccacgacgatatcgcctacttttatcgtagcttctacgctagatggcaccgttacctgtacttttattgcaattggccgggtggatgaatttgtatagataacaccgattgaccggtcggcggtgacatccatatacgcttggcctacgccgatcgttacatcaccggcggcaacagtgccggatgctacgcctacattgagttttgatgcatctcctaatttaagatgcgctataatctcatcggcatttcttttacccagcaattcttcgctaaaaacgcttaaatctgccgtttttgcggaatctttgcccgttaacaccaggatcttattagccgcggcctcaagaattgatatggccgcgattattggtgttttttccgcttttttatttagctcggcatctacataggtcttgtctgcctttttgtctaactcggtatctacgtaggtcttgtctgcctttttgtctaactcggtagtgattttctggaagctgctgcccgtatattgtacgccgtctatttcgaccgtgatatccccggtggcagaatagaactgttttagcgcgtcggattgctcctgataacttcgcaatgtcgcggcgagctggcgggcaaagtccgggcgtgactgcccgtagaagctaagtattgcatacgcagaccctgacggtaagttgctggcgtccgacaccaatgtcatcgacgtgtcgccgtttacacgcgctatctcatagatttttaccacaccggacgacggaacgagaagcgcctgaccctctcctatacccgcggatggatcagcccattttgtacccgaaccggttacctctttgccgtttatggcgattgttcctgttttgtaccatgccattgttatgtattcctatggttagccctctaaaatttcggcactaccgcgtctagcccgccccattaataattgggcaccgtcatatgtgagcagtgtatgcggtttttccggacggattgtattaggccaagtcttggtgaactctattcgtatccatgtgccgttggcaccaggcggaagagtcaaacccgatagattaaatttatattccgccgtttccccgttagaccataattccgtcaagacttttcgcactccggaggattcaacgtaacatgatattctagtcataccgccactaccggcaaaaaactttacatagccattggatataatggtctgttcaaaatcttcagcggggaccttaaataggactcttgtggcaccattgcccacgcttacatctttacttgaaaatgtaaagtttgagtattgtactacgtcgccaactattctattggcgtgcaccgtccctttaaaatcgccattttccgcgtacacagtgccgcgtatcgtggcgttattgaactctacggccccagatttattaatagtccagccggcgccacccggggtgtagtcatccgattgaaggacgcccgcaattttagcactaccgatcgaggcgtcacgaatgaatgcgtcacgcagatatgcctggtcaccaaccaccccaaacataagttctggtttatctcctactttagccataacggcaaaacgatcggccagcatggcgacttgcgtagagacctcgccctctttcacctcagcggaaattgacatgccagcagaataatattccccgttgtatgttatccccgcgttagtatcccataccgcgtaaccgttgccgtcgtaatcgaacttcgctgtcatttttgttgctacggctgcttcgttttctgccaccgaggctttaaccgcagttaacgattcggccagggcgccaacttcattcaccacgacattatcgattctaacgatctctgcggatagttcgcctaattttttggattttcgttgtatgtcgccgtcattggcgattgcgttctgctcaatggcatctatcgcggcattaatgctcgtctcgagcgccttacctccttcagatgacatgacctgatccccgagcgcatccataatcgaatccgtgtcgattgatgcccgcccctgtacactggcgccccattccgatttattgccgaggcggtcaactaaacgtgcgcgataccaaaagacggcattagctttaagaccactatgggcgaaggacttgctcgggtatggggataaactaagcgttgtaaaggcttcttcagtgtttgtcggtgcgtattggatctcagtgtattcggtgtcctcggcatctttggggaacgcccacgcgacatcaataccaaagactacatcgctagatgctctcagggatactggggcgggtacttcaccctggcggccgtcgatatgcgttaatacactggatgcccacacactagacgccccgaaagacgtgaccgcacgaacgcgcaccagataatcgccggcaaatacaccttgtacctcaaaaccatttacactgctcgccggcacattaacccaatcattggcccctctgcgccattgtgcttcatacgcgacaatatccggctgtactttaccgtcggccatgcgcgacggctcccaggtagcgcgcatcgtcgttacacgctggttttgtcgcacctgctcgtaactggtgatggcgaccgcggtcggtgcgttgacaattccggtcggcaaaaggctaactggtggcttgtctaacctggcgccatcgtctacggcatcatatttcgagtcgttatactccgctgcgctaatctcgaacgtgttattttcgtcgtcgaaatctagcgttaacacgcggaatttttgcaacgccaattcgcctgagtcaactacccaaaccgcatttaccgcgggcgctgccgtgaatggctcggcgatagttacaacgctatccccgaccgaagcaatttttctggactctaccgcgccgccagtggtgcggatcagcaaatcatcgcccggtttaccgtccgtagtacgatcgagcattacctgtttggttttctcgtcatatcccgcgacgcggccacccataacacggcccccgatccgctcgtcggccaaggcgaagaccgtaccgggcagaaaggcgaaaccctccaaccctacgcggagttttaccagccgatctaccgagttggtaagcaccgcccatgatgcgcggcgctgcgcttcgctttcgcgcgtacagccgatcgctgtcagttgggtctgttcgaaaccgagttgcgccacaagctcctggaacataacggccgtcggggtatctgcgtaatggttttgggcgtcactataattaacaagagccgagctaaaacgcgttttacggctgccgcttgaataggttggcttgccgatgatagaggcgcgggtgacgatctgcgatggtgctttcaccggcatatcggaaaccacattgaacatgttgtttccccaatacgtcagcccgttaaatcccgccgcgatatcacggatcaccgtccaggcatcggcctgcgcctgcaaataacagttgaactcgaaacgcggctcaacaccaccggcaccatccggcaccatctgatcgcagcgctgtgcgatgcggtacaactcccatttatctagcatggccgccgtaacgcgcttaccaagcccgaaccgcggttgagtcaatacgtcgtaccatacccacgccggattgttggtgtaggcccatttaaacgacccatcccaattgccggaataggtgcgggtgtctgggtcatagttacttggtactcggataatacgccctttggccaggcacgatattttcgggatattctggaatgattttgcgttgaactcgataaacaacaaggcggtatgcgggtatcgaaaacgtgcgtcgataacctccgtgatggcctgaattttaagcgtgttggcgagacgtgcgctatctgaatccggggtttcacgcacaacgcgaatccgccagccggacgagctacgcggcaggttgacacgtatcgaccgctcatagagcgtagttgttttacctgagatggcgaatttaccctgtgtactaaacgtgcctcccccaacggcaagatcaatccggaacgataccgaagtgccgactacgtcgccgtcatcctcctggtacattagcgcgggaacgcctacgcgaacgacgacagcgtcgatctcggttctggttagcgcatgcacccacgggctggcctgtttaatttcagtaccgaagcccgtctcgttttctactgcggggaaacccgagatcgggtcctgctgctgtacgccagggcggacttcccaacgcacgccgtcgaagttacgcgacccgtccggattgcctagcggcgttccgtccaggaaaattttagtgtcatccaaccccagcgccatttccccctcgcctaatgcgatgagtagacgggcctttgcctggctacgaatactatccggttgctctaccggtgtatgcgaatcgccgccaccaccctttgcgcctttgatgtaaaaaagttgtctctgcattatgctacgtcctcggcaacaattccggcactgattatagcaccgccgatctctctcgtcccccatagcactccgatcgggttacccattgccgtggtgttaacagggccgccgaaagcgtaggatggcctattatccggatcctcycgcgattgtaatccgcgcggctgcggggatagcatctggtaaaggcccccagccataacggccgcacccgcccaagccatttgcgcaccgaatgacgccaacgcaccacctgagaaaatcgtaagacccacgcccgccacgacaagaacggcgccgagaatggtctgaaatagaccgccttttttcgatccctcgatcaccggggcgatgcggatctcatcgttgcctatgccatcgtgcaattcttccgcgccgatgtttttacgtccgcgaaatacggcgaacgtcataccgttctttttggcgtgcaagagatactcctcgaatccatcgagcgttacgcacagcgcctttaccgcttctgtcgtggtcgacaccgcgcgacgatgcacccggccaaatgccgcgcccagtttgccgtacaggcggattgtgattaatctctccacggttttaattcctccggtaaatctcgatgacggatgcataacacggtgcggtccacataatacccgccatagggtatctcctccgacgcgcggccatataggtggtgcaaaagcccacactcggtaagtacaccggcatgattcggaacatcggcccgcacctgcatgatgacgacgcagccgggcgcaggatccgcctcgacgaaacccgctgcggcccaattatccatgtataggttttcgccgcgctcccaccaagggtaatttacacggaaatctaataggtcgatgccttgccttttatgccaggccatgactagcccgtaacaatcatcggcgcccaacacgaacggccggccgattagcggcggatcctgcggtgagatctcgcggtactcgtcgcaatctggcgcatataccccccatatgattccgcttgcattacacgcggccttatcagcatctgatggctctgccgtcgccccgtcgcccgggtgactgtgtatgacgcgcacaatgtcgccctcgtcggcagccgccgcaaagtcagccgcagagatccggaaatgttctgtcggcatttcatgcgtattggtaactctgatatatcgcagcgcgcggccattttgtaccactaggccgcagcattcgttatagccagactcggcggcgtgttttttgatctcgcctagaataattttgttcatggcatcaccggttgatcagttgggctgcaaggaatccgccaaaatccagtgctgcggccttggggtcggccatatcggccccgaagcgcttaacgcaatcggagtaacacccaccacagcgatcaagcgccgggtctgaaacagggttgcccttagcgtcgaaatacgcgttgccgttgtaggtacagccgtcgccgctgcggtactgtccgcgcatcgcccaggtgcatagcgatgtgatctgacgtgtcggaatttttagcccttgtaaatccgccggactgcttaacgtccaggtgatcgactcgtcatcctcggctgatttagtatcgacccaaaaagtttgataactgcaagcgtccggatcggcattggggttaccgtcggggaagttgcgcgcgtcaaggtattcggcgtaggtgtaaatcacacgtactttcgcgtttatcaagtcgcgataatccagacataagcggctcaaatacccggccaagttcgaaaccgtgatcgtaggtgtcgcggcctgttctgtagacagcgacaacccggacacgctaaacggccagaagtcgaacattaacccgccaaaatatatcggcttcggccctaacttagcctcgtcgccggccgccgcttcgatctcggcagcggagtgcgggaagggggcataatgcatgcggtgtacgccggccccgaactcggaggcatccacctcgactaaaacgatacgcccggacgggtctagttttgccgattggtctatgtacgacgtcatgcgaacaccccatacgcccggttcagcgtgaaggtaacctcggcggtatgccccgttaggttaaacgccaccgattcagcatcgacgcgatacagccccttttcttcgccgggcggagtgaagataaatgccttgacgacgtgccgtaagaggaatgcccgcacctcccgcattcgagcgttgtcaccagcgcaacgcaaggctatggtatcggcgacggagttgataccgttttcggctacctgttcgtagccgtcgcccatcttggccgagcgtaccgattttttataggtgatcggcgcggtcaattcgcataattcagtaaaagtttctacggtcatctaaaaatgcctccgctatttctggaggcattttaacaaacgttggttgcgctgtcgaatcacgccatccgcgatttgtatagcaacccgcccggctgtagcatacgcgtaacttctgccgttacggtatccttcatctgcttggcgatcgacgcgccaagggcaccgccggttgacgttgctcggcccattgcaccaccatcagaaaccgtaaccgagatgttattaatgatcgatgaacccgttccaccttctgccgatatgcctaaacgtccatcggcggtgcgcttcaacggcataatggcctcaggtccggcttctcccatcaacccggcgcccttggcgaacttcgggacggcgtcgaaattaaacatggttggctggtttaccacctgcccagaataccgagaaagatcaccaccggaataaacgccgcctttcgcgttagcgaacaacccgcctagccccaatgcattagcagccgatttgattccttgcatcactaatagttgcgttatgatatcggcgatcatctttagcacgtcggcggtgaacgacttaaaatccattttgccggaagtcacaaaatccgagacggctttgacaccgcgattcattgccgattctaccgcttgccccgcgatatccgcgtaattcatgctggcttcggcccaatccttaagccctttgatcgccccagcttgccagtttttatccgcggcgtcctgctcggcccataatttgcgctttgcctctagcgccttctcgatatccgccgttgtggcgccctgagcggccagtgtagcacgcgttaccgcttcatcgttgaaccgcttcgcctctttggtgctcatgccgtaggtggccgctagggcctgcatttgggcgtccgtctcggcaacggcttttgacacattatcatgagcctcagcctggcgctgcagcacaagcagttgatcgccggcatccgccagttgcaccttagacgcccggatctgctcatagctcgccatgatttgtttttcggcaagtgtcagttgccgtttttgcgaggcttccaccaacacgctatgctcggcctcaaatagcaggagagcgcggcgctgctggctggcgttgcggtcatacgtgtcacgctgcttcatcaatgcgatctgcgcttccagcgcgcgtagttgggcggctgactgctcgtcaacacggacgccggcatcgactttaacggccttggcgcgtacaggcttttcctcatactttttgcggatctcctcaacccgacgattgtactcggccaggctgatcaggttggcatctagcaggcgcttctgtgtgtcgatctcacggttttgtttctgcgtgttagtggcgaacttctctgcctccgatgccgccgtcgcgttgagcacgttttgtttttgctgctccagcgcggaggctttgatcgccgcagcttttttatcctcggcctgtgtctgctgccgtagcgtttccacctggcgccgggcagtctccatctggtcatatagcgcggcctttccggcctccgttactaccttgctatctttttcaagctcccagcgctttttatacgccgtgtataccttctcggcagcggccaattgcacaccgaccgattccgggcgcccgatatctaagatagcgtcccacatattcttggcggcgttacggatcgacatcatagcgagctctaccatgcctaactgcccggtaacattagcggcaagatcagtaaatgacgctgaggcgatgcggttggcctccgcgatggcccgcgtttgctgcccctcgtcgatcagcttctgaacacggataatctgctcttcggtcaccgccttgtattgcgtctgcaaggcgcgtaaaccgccgaccgggtccgttgacagtttggcaacttgcccgattacatcgtccagcgattgccccgacgctttggcgaacgcctgaactgatttgcccagtgcgctaaaatccgcgttggcggatacaccagcggcggcgagcttctgcactgccgatactgtgccacggaaagacccgccggagcgttcggcggattcccctaatttaagaatttcattagcagttaggccggagaaatggctcgtcatattcagcgtcttattgagacttgagatctgacgatcagcattcgtcaccccggcaccgatagtggcaagggtcgcacctacggcggcgatggataaccccaccgggccgattgtggacgctagggcgcgcagagcattgccgatgccgccaaacatatcgcgcaattggccgccctgctggataagtaccgttaacgggcgctggccaccctgcaaggatgttacgatgtccgtgatctgcgccggcacgccgcgcatggtggcagtgagctgcttctggctcatgccgaatttgtcggccagttctatctgccgttgctgcgccttagccgcgcgctcggtggcggcggaatttttctctaatgcggtcgccgccgatccagctgctgcgcctgcgccagccactccggacccagcctttttcgcggcgtcgcctaacttatcattagcccgttcggctttttccgccgctacggtaagtttgtctagctcggtgcttgcttttcgatacgctgccgacgtcggcgcgcaaactgaatgcttgcgatctcttgggtcatgatccgccctcccgtgatgttgccgatattgtacccgctttatcagggcgcatgcatgctcggttatttgcataactgcgtatgcgctttgcgcttcgtgtaatcgagtagaggacgaggggtggctgaatcacccccgtctcctcttacgaagtaccggggcgattttggggcgggcggcttttttctttgtaaatcaataactgccccaggggcgggcgctcggggcgatttgttcggggcgagttaaacgggcgttattttattgttaaattcggtgttggttgcattgttggttgcgcttatttattcaaatggttgccgtaattattcatactataaaattggttgcattgttggttgcacactgctatagttaattcaacttaacaggaggagcagaaatgattaaatctaaccacgctgacgcctacacaccggcggcgcaatcactattggcgactcgttcgaaagagtggctaaatcggtcatacccgcgtaacggagaggcgatcccattctatccttacggcagcccggtttatcagttcaaccagttattgcgggaggtgcgccggtgatcttcgataccctagaagatgcgatcgattacgccaacacccggcgcgcattatccttcggcgcgtcacatggggcccgccactactgtgtgtatctcgacacaagggaaaatatgcatgtcgcatcccatgttgatctgccttacatcaagcactttaaccgcgtgatatggtccacccgtgcagaagtcggtgctactgggtcacgggggaggccacgcgcgctcacacggtgtcaggaagcggaattgcggcgtatgcgtaatgatgggttgccgtatgctaagctcggcgcttacttcggcgtaactgacatgacggcgtttaggatttgtaaccgccgataaacaaaaagcggggcagctaccccgcttttttcatttcttctttcgccacttcctcaagaacgcggatgtcgtctagcgcctggcgctggtttgtgatgccgtgaatgtcgaacatccaccgcaggacgccatagtcgagggcgtaagccccgcacggcccaacgcgccattgcccggccatcttggtgaatatcgacaccaccggccatacgtccggccacacttctacggtcttgattaactggtccggcgaaacgccatatagctcttgcgcaatgtcttcactcggcgggacggcatacagcgccgccgccgcctctcttagtttttttcgcggaggcactgcaacgttgtagtgtattccttggtgatctccccgtacgcccgcgggaagttcttcaccaatagcaatgcgttttctttattcagcggatcaccaccatcgacgcgccagtcggccagaaggtatagcaggctctcagccatgacgtcgtaggcgtccttatccgggtcttgtaatgcttcgtaggtgtcggccatattacgctgatattcatcaaccggataatgccggaaaaccacatctaacggcacgggcttatcctgtcccgcgtttggcaccatgaccgtggcggggaacgtcggcgcggggtttaacttaaacggtgatttactcattgtgcgaagtcctcttaataaatagccgccatgcggcggccttagtatggcacggttgccgttacagtgcatccacttcggatttaatatagatgcgcataccggattgcatcgatagcgctactgacaccgtttcgacgttgttaacttccgatgacgggatcggctggaatgacaccttagcggcatacaaagcggatctcgcctttaccgccagccgccgccgggttgatgaacttgatcgccgcaaaagcctgtgtctggtctgctttttccagcaccgggcggattgggtcctcaatatcatgggttagcgtgtaggtattaacgatcgggtttttgaacgtgttcaggttgatcgcctgcttcgattgcagcggctgaaatgaaaccgtctgctggtcgccgccgctggtagctacgttcgtgatatacgggaaatcgacgaagcccgagatcttgaccatttcccccggtaccgacggagaaaaggcggacgccggataataagtggtatccgtggtgtcgagttttagcatggtgatcgttttctgcgccgtgtctacggccttcacgatcccgcacaggttgagcgcctgagtccacggcgatttagtaagcatcaccgtatcgccttttttcaccgccgcgcccgctacggagcttgattcatcgtaggtgaaaacacattcagccgcattggtcgccccaatcactttgatcgggtccgacagcgtagcgcccatctgcacactactgccgtttggtaattgataccccatgttatgatctcctcatagcccccacggggcgattaagttaacgctctgtaggttacagagatagggatattataccccgtgtctgtcgccagtccattaaacaccgaagccgggcccgaaacaccaacggcaaaccccgaggcgtcgcgcagcatcagtgttgccgggaatgccgcggcgatgtcgtctgcaagtttttcaggtgcgcgtgttccatggcccgccggaatcatcgcggttatttggaaaataccctgatagatcacatcggtttgctgtagcgatatcgaatcactctctgctggcatgaggttcggcactaacataacattggtcagcgagggccgatccgtcggggtgttttcccacgctaccagaatatcgccgagggggtatttcaaccccgccactacggctgatagatggccctctagcagggcgcgtatacgggatacgctcattgtttagattctcctattgcctgtgcaatataacggcgtgcttccgcagcggttatgcgcacgatcccgttaggggcttgattagaccaaccgaactccaggcgacgcgcatacggcacattattgcaaaaccaaacgctatgcacttggtccaagtttagcccagcaagaacggcatcacccgccgccagtgtagccccgcccgatttatcgatgcggtcgattgccgtatctatcggggcgttaaacgacacctgccagttacccctgaatcggccgcctgtatatccgcgtcccacttcgcgccgctgaatgaaggccaccgttttaccgttgcgcgtcttgaattctctgcgcactccggcatgcaccttttgaccgcgctttaatcgccccgtttttgtaactctacgcggatctttacgccgcatagcgttaatccggctggcacgtgctttagacttagacagttcggcattaactttccatcgcgaggggtcgccgaccggggataacgtaatcaggcggcccagtatcttcatgccgaaggcccgcactacttgatccatacgctggtttgtcttatccgcgaaaagccgtaccgactccgaaaatgcgcccacagtcaccccctaagcttcaaattatatgcgatcactgtcaagccattcggtgctgccggattgggggtgatcacgcggtactgtttccctcgaatggtgaccaggtcacccgtttttacttccgcaccggcaccagacgcagcaaaacgcatatcgccggccataatgcgggtgccgtctacctccatgggcttgtattgcgtcaacacgccagtgacggtgaacgtttccgctgggtggataatctcatctggcccgacacgctccacccacgacgggcgggtgacggttgcggccatgccgttttgtgataaaagccgatccgctgtcttttgtagcttacggtaattcaaagccattatcgttatcctcgacgttttccatcggcggtacggcggacgtttgcggtaccggatcgaccggagccgaaggcggcgtctgttgctctgccggttgcaccgcaattagcgctgcagttactggaatgctaatcatgatcaccccctcgacacgctaaagccaaaggccatgccgtcggattccgtccacgacccgatcaacccatcaagccacggaatgtccgcgcgcatgccgatcgtgtccttgtcgtaggtcacggataccgcattggccgccgacacactgatctcctgtgccccgctaatgctcggcataaggtcgaacccgtcggcgcataatagcgccagccgataaaccgccgtaaacacgtcctgtggtgttcctgtcacggtgcgcgggtccacaccttccggaacgtcgatcggcgtctctgtggcgtccagcagcgcacaatcaccccacgcgatgccgatacgtggccaggcgtcggcctgatatttgtccgctttcttaccggaccattttttcgagttgatgaaatccgtagcctttgtcagcgcgatcgctgcgtcagcggtagtgatcgtcatcccgcgttccgcggcgaacgctaccagtttttcaggtttacccaacatacaacccccaatacgaaaaaggcggggcaaaagccccgcccctagtccggcatagtgccgggttatgccaccgcacccgcagtggtggcggtaagtttcatcagtacgccggcggtttctttcaggttcttaaccggtaccttttgcgcaccgcctgaatttttcacaggagcgttatccacggcgccctggtccagctcccagtttttgtaagatgacacatcagccagtttagcggagcgcaggccctcgatttcagcgcgcagggcggattttacgcgatagcccttaacggcaacgttgaaatcaaactcgccctgccaccagcgctcgatgttttcgttgccgcctttctgttctgccagcatgtcgaggctagtagtcgtgatcgcaaccgcgcccggtaccagaccgataattgcatcggggccgagtttggtcgaggtgctggacgcaataacatcggcaagcgcagtgccaaccgcatcagagatcaggaagcgacggccaaggccatcttgcagcacctcgatgttgccgatcgcgaagactttctctgcggacggtacggcctgataggcgataaattgcgcccattgggtacctgacatcgcccaggtcttgatgttcccagcggcgtcgccgaacaggctagcggctaacgggaagtcggccagggtagggaacttcatgccggtagcggtaacgttaacgcgtgccggttgggtctactgcgatgccgcgttactgcacagcgcgccaccgacggcaccgacggcacccttgatataatgctggatgatcgcttctgtcgccagcgcggatacttcacccgcagtctggttaacgtccgtttggatcttagccatcatgccggacgtaatagcaaccggacccactttcgcagacaggttaatgctgttggtcagcatgcgggccagcactttggcgtcggctgcagtgcctaccggcgcataggcgttacgatcggacaccaggccgtcgataataccaacggtgactttttcgatcacatctttcagcacggtaccgctacccatgacgatagcgccgccgctcgacgcgttccacacgttcagattatcggcaaccaattgaatggtagtgccaattaatttttcctgaaatacaggtaagctcatctttatggccctttaatttatgggccgtgcggcccggttgtaaccaaatgtcaaacacataataccacaggtcgcacgacggccaaattattcgccgataccactagcgatctcggtcgcgcgttgcgccaaggtactctgcggcatagcgccgccgccagcaggctgaaagccacctttaggcgtgccgcccgcgcctgaggcaacaacaaccgcggcataatcggcgttagtgcggaattctttttcgaggtcatccagtgacatcgccgacggtttgccgtctttcatcacgcgcactttaaaatcgccgtccacctcttccaagctcaggcgcggtgcaacatggggaagcatcaacccggcatttttaccgaaaacgcgggatgccagatcgcgcgcggtggcgccgatcgtcagatcctgcactttcttactcaacaattcggttcggccaccggcctcggcttgcactttagccagtttttcctgccaggatttatccagcgcatctacgtcgccggccttgcgcgcggcttcttctcgcgcttgtttctcggcttcttctgcggcgcggcgcttctcggtttctgttttcttttcattcagtagcgcggctacctgtgattttaaaccggatacgtcttccaggccttcaacggccaatacataatcgtcgccggacttgttgtaaagccccttaacggcatcatccagcgcgtcgaacgctgcggaatcgatcttaaacttcaacataactttgccccctaggcaaaagttaaggcagggccaccccgccttttcatcatggaatactatcgcctcgattggttgcgtgcaacttagtcacgccacagccccttatttttaagctgcgctatggtgtacaactcgccactatcgttgaacatcttcggcactttaagcccgcgcagaatctgatctgcgcgctcgatgccgtatatttcttcgaggacgtgccgcggctgccgttgtacccactcgaaataggtcgtgccctcggacaccgattccgacatatagcgcccggtcgcgggatccgtctttagcgcagggcgtgttgaatcaggccaatcatccaagccgcgaataacccatgtctcggtactccggcagcagtaatgcaatttacccggcccggcgccgtactgactgcccgcaatatgctttttcagttcacgatctgccgatccttcggtgtccgctttcacttttagcgggtagaacagccgatcgcgtaattggcacatcggcgatgtatgcgtgtcgagagtggataaccaccgccggcacttcactacatcggcattggcagacaccatcaactcgcgcgcggttgcgctatagtgattcacggcagatttcaccaccgaagacacgccagatgagaatttaccctgccaggtggcttttactcccgcgacgatctccatagtgggtttacccatcaaatagcccgcgcgcacctgattggtgatcaagcgcttcgcccaggccgccaggtcatcaggccatgacagtagcgtattaccctggaacggcgcagagaacgccgccgcggctacctgcgcgcctgtaatggccgcgatgggtactaccgcacctggtatcaacgcggggcgaattgccgttgttagcgcatccgcgtaaaacgatgcctcggtgtcggcgaactcttccatggatgtggccagctcggcgaaatcatcgcgcaaagcctcgctaatcgtgctgtctacctttcgcagggcgcggcgtaactgcgccggggtgattactgcgcctctcaccttctccgacgcaagggccgcggccagcacttcggcaacacgcggcgcgagccgtttaatcgcgcgcagcactttagccgcttggccattcgagaaacgctggacgaaaatatgccggcggatcagcagatccgccagcttatcactggccttcatcattcacgcccccggtcggcggttcattctgcacgcgtagcgcctccatgatatcttctacgctgcgcgaatcatcctcaataccctgacgcatcagatagcgcacaaatgattccaacgtaatgacaccagtttgcacaccggccatcagcgcggtgatcgcctgcgggtcgatatttagcgctgtataggtgcggtctaacatcacggagccctcaccgacaacaaaacgcccggcgatctccagcgctttgttaaatgccgcctctacgttgcccgccgccagggctaaggcagaattatctgtccgggcatcaaaatcggcctcagttgccgttttaggggcggatccgcgctcaatcaaggcggcaccgatcatcgccatctgtttttcgcggcgctcgcacagggttaaacatacgtttcgatcttcggcctgcaacaacgaggccgcgctatcctgcggcagtggtaggccgcgagtagcccctagtgcgatgccgttttgcaggtttttatctacccaagtctgcgtaagtccggagactactaacgttggctgcccgactacatgcgccagctcggctaaatcggcctcggcggcgaaatgcttgatgttcagccccgccaaatctgccagcggcgccgggtcaacactggcgttattgtcgaatgccccgccccatgcccagggcaacgcctcgagaggcgatccatcacggtcgcgcaacggcacaaggtcggttttagtgaagccggacggataggcgccaatggttgcaccggtgttgtgaatccagcggcggcaatgtgccacgccctcgacgagacgcaactcgacatactcggtgacgctgtgcatagcgaaatcatcgacatcatcgggcatcacctctacggtgcgttgtgttacgacgagcgtagtccgcccattctcctcgcgccagttgatcacctcgcgggccgaatacaggtcgatcaacacgcgttgccccgccgcctcttcggcggtcatcggtaccggattaccctccgcgtcgtattttgccggtctgctgaaatctaccaggaagccaaagcggccgcctttcagcgcggcggatagtgccccgcgcaacacctgagcaatcggcagccctctgccgtccgcgttctctcgcagcacatccagcgcaccagacaggctaacctctaccggcttggcgaacgcgacgcccaatagcgcttgcagcgtgcgcccggtagcgttgaggaacggcgcacgggctagataactgtcgtagcgcttcgacgccatcgggtcttgactcgggtcaaaccccggatgcggcaggtacttcgtcttctgtgccttaacggcgcgctctcccgctacgcaatcgtcgatccgcgcccactccggcgcgtatcgcgcataatccgggtgtttggtatccacaccagccataatgataatcctcagtagaaattaacgacgatcgaatcgttggccgcagggcgaacaatcggatgacagaatgcaatcgggtagccgatggcgtcggccatatggtcaacacccgacgttttatccggcgcgcctgttttcgggtcccagatttgctgctctagtgcctcggtagccttggggcatcgagccacgttcacttttaaacggcgttggcctttgccgtttaggatcatgccgtttacgtcattgacgcgatcgcgcaccgccgggttgacgctattgtatgacaccgtaaagcctgcgtcctcaagcatcgcaatatcggacgtattggcgttcgttgtcttgcggttctttccgctggagtccgggaaaatctcaattaacccgcgcgcatggtgatccgggtatcgctctttaatcgcgtcgatcatggcgtcggtatccagtaagccgcaaaactcgtctaccaggtgcatttcctcgacgccatctgcgatccgctggacgtacaccgcgccggccatctgcccgacgttaaagtccatgccgatcattaaggtatcgtcatcctcggcaacggtatcacagttattcagcgtgcggctgaaattgcggtatacagcgccgccggtcaggttgacgaattcaccgtttaggtaagcctcgatgagctcaggtggatattgcgcccgcagcgtgtcgatgtagtcctgcggcaaatagtgattatcagtggtgcgggccttgatcaggcgcttctggtcgtccgcttccgatatgaagatctgatacatcgcccggaatccttccggagtggacacgatcaccatctggcggacattaccagtacgcaaacgccccagcagtttgcggtatgcctccatcgcaatatctggcttggtagtatcgaactcgtcgcacacgcaccaggcggcgttaacaccgatcaggcgggtgtagttctccatcgagtcgcagataatgcgcgtcatctgccccgcaatacggcaatggtagatcttatcctgcttgttgaacttccattttatcccagcttcgtttaacgccttctccagctccggatacatgatcttgacgagtagcggtattgtcggctcggtgataatgccatcgcaacctggattcaggatcgcgagttgtaccgccttgcgtgctgccgaccatgttttaccgccaccgaagcccgagcacaggcctagtattttggtcgtcgtatcgttgatcaggccgcgctggtgcggcagcgtcttgatccgccatgtgcggcttttccgctcctttttcaccggcgcggggacaccaaggcgcattacctgccgctcggctttagcaatacgcgccagcaattcgcggtcgaagtcatccatcacgcgttaccgtcagcttctgcgaccacattcacatcggaatcatcctcatcgctaaccgtatcaggttgctgatccgtgatgccgtggtttgctctcatctggaagatcgcccaggcggacggcatcagacccaccccggcggcctggctgatgaattcctgttgcagttctttgcagatggcgtaggcctctgcgaagtccggattagcgcgcgcccataacatcaggttccagcgcgtcaccccgatcaggcgagcgaattttacgaaggacggctggttatcgcgggggatcacctgggcattgcccttgtcggtgtagttgagctgccaggcggtcgcattctcgaagtattgcaccattttctcggcgtactccgggcggtagttggtagggcggccgtacacgtaaccttccggcatttcaccatgacggcgttttgtaggcatgcgcaccccgtttactttaggcttgagctttgcttttagtgccataaggcccccatgttgctgctctgcggtggaccatccaaacgcgagaggcggacccctggcccgccccttgattatatagcgcgtagaaaatggcgcaatgtagcaaaagtcattgcaacgcagcagttaaagtgctttaatacccttacacaaaagctctttcccataagccgtaggtgttccaccaccgaaagctacaaaaaggcccgcattatgcgggccttttcttttgcgtcatccagggtacacatcccggcgtaattcgaagtgcgggccatcgtaaaagcgctcgtcatcgctgcgcccgttctgattccagtcgccaccccagcggatcgcaacgccctgctctttggccgccgcaaacatcgcatcggacactgacttaaatggtgcgcggtcgttccacgggatagcaccggcaaccaacggcgcgcagtctaccgcatgcccggttagatggcggctgttcatcgtctgcgatttgccagtagcaaccatttcgcgctgccgttctacggaccgtcgcccctcgatcactgtgaaatcgaccggtgtcagttcgatcgcgcgcttcactacgcggactaggtcagggtgtacgccgtccagccgtgacaggctgcgactgcttagtttgaacattattcctccacgggatcataggtaatatggaaaatatccggtttgcacggataaaattcacccttcacgcccttgataatgtaatccccctctgttgctacgtgacgtgcgcgcttatccggcccatcctcaagggttactacatcacaaaatgcaacggcgcccgggtgtctttgtttgcgaatgtttttaaccgcgcctccgcaccactctaaaaatcccgcggatggcggataagtaaaacgcatagcttctattttaactggctttttcacaaaataacgtcggcattttaataccctctaaaagttaattcgatttaatactagcgcatttcttgctgtttgtcatagctcaattcgcagagattggccgcttcccaggatcgatcggcgaaagccgccagctcttcatttcgtcggacagatttttcgagcacgtcggtaagcaatacggcggtttttccggtgctcgagccagcgggctgagagtcggaatactgcaagatggtgcgacgtatcgcggcgatttctgcgcgcagcccatcagcagtacgacgggcagcgtcagcatcggcaacagcctgattgcgttctgccagggcccgcgcttcgtcttcagcctttgcggcgtggcgtcggtcatcttccgctttgcgcttcgccagggcttgtaggtcccgctggttctgctctgctcgatatcgttcatagccgttcgtatccccccgctggtatagccaatagccggttgccgttaagccccccgctagggccagcccgctcaataacgcgatgatggttttattcattgcgcaccttgcccttggccctgctcacgatgcccgcccactttccgcaggcgggcttccagaatgcatcccggataagctcccgcgccatcatgaacgcgctgcacaccagcaatgtggtaattagccgttctggtagttcgcctgtctgagagtgcagcaggccgccgagactggtcacggcagccgccatgtacagaatacgcccgattacgccgtcgcgcacttttgggctgtagatgccccatagcgccatgaggaagatggccaatagcgccaccgagtagatcacgttgtgctcgatcatgattattgcccccgcttaaaggccccgattaggtccttccatcttgcgccggcgatcgcgccaatgtccagcgaatttaacgtttctttaacacgctgcaaaaggctcatgccgaacacgccgatgagaaagccgatagtggcgatactgcgctcgtcagtcagtgccagatagtggccaaccggctcgctgaggtagaacgcggtaaccacaccgccgaggaggaaacatgcgcgctgccggaaggtgcggatctcggtatgcaccaccaaccccacgattgcgcccataacgcccgccaggagcacccaagacgctgatgatactttctgccataagtccataagaaaaggccccgtaatgtgaatacagggccatggtaacattcgatgcgttatcgcgcgattttagcgtttaaacgcaccgccgatgtgctgaaacagttcatcgattttatcgttgatgcgatagtcacggctaagcttcttagcatcgagggtgccgttgataccaattggttctacgccggctttcgcagcgcggcgatccgcgcgttccgtaatctcggcaacgatatcgacggtaaaagcgtgcgggccggacaggctttcgtattggcgcgttgccgtctctacctcggccatcgcgttattcagacggtaaccggctttcgcgtactgcgccaggcgggcgacggctgcgccatagaaatcatctgttaaatcacgtccgtgatcaccaaaaactttgcagtaatggtgatggacatcccgcgcgagccgctcatacttcccatcagatgctgcgagcagttcgcgggtctcctccagcgcgcgcagtcctagatcgacgggcatagctatgcacggctcgcccgtagcgttagagaaaacccccgacagccgatcggcctcgcgctgtttcagctctgcgatcagcgagtcggcagcggccaggcgggtgcgaagcgcctcgttttcggcgtgtagttctttcgtggttgtaacagccttatccaggtcaatgaatagcgccgattcaaattcgttcattatctgctctccagtatgtaaattgcggtgtggcgttatgccggtcaccgcgttgagtatagtcaatttaacaatcttcgcaaggcatgttgccgatccgcagtaattcgtcgatcgtcgcctgctttttggctagctcctcttcgagttgccgattgcgctctgccagctggtggcgctcgcggtcgcagcggttgaacatggactgtaaacgggcgtattgcgggttcattgcaatctcccgtccatcaattccagttcgcagcgcatagagtcgagctgccgctcaaggcggcggcgttcggcgttatacatcttggcacgcggcagggtgcgccactggtagtccgccttttcgattgccgattgtaagaatgcgcggcgctttgcctcgcggcggtacttcatgatggcccgcataggagccgtgatatacgcgataagtttttgcatggcgctactccttcggcaatctgatcatcgcgttaaggtcttcgcgcgataaggctagtcgcgtgccagttgctgcggcttgcttctcgggcagcaggtctttcgcttcgggccaggcctcgaggagcttctcgacggtgcggaatttgctgatggttgctttaaccgaggccttgatctccgtatagcgattgcgcagtgccttatcctcggcctggaatacggagaattcatccagtgccgttgtatccgcgattgggtaccacccggagttagggcaaaacgcttcctcatcgaagtcaaccatgctcccctcatagagtggatgggcttcaccgcgcccattgcgataaagatacatccgcaggccattaaggttaacttgtacgtagcctacttgataaacgacaaaatatgcgttctggttagtgaaacacttcggtaaacccagctcctctatatgcttcaccaaggcccgcagcgcctcatcagacgttttgcattccgctagggcgcgctgccgggtacgttcggtgaagtcggcgcggcgggcgcacagatcgcgttcttgttcgggcaggccagcctgcaccagtgcgttagctacgatcgcgtcgcggattgctgcattcatacgtgttgccatttctgctattcctcgttgttgttaattcgaattaactatagccccgctcgcggggatgtgcaagcggtatttactcaatttaccgattcgcgcccctgggcgcgccaccaggttaggcgtaagggttgttgtctttctcaaagcggaaggcgcccttgtactccggcatggcgtgcatgattaccatcgcgtcgtaccatgctttcattagcggagccaactgtgccccgagcacccgacgatcggcgtaggtaatggccgggttttcgaactggcggtttattgcgtcgatatctttttgcatatccgcgatcatcccgcgctgttgttgatacgtggtcatttctgcaatccctctttcttgttaagttgatttaactatagctcgataaaatacggattgcaaggtgcaggcgcgcaaaagtgcctagttaccttgcgtgacaattcgcagataacacaacgcaatagccggcgataacacatctgtacttgcaaccaacccgcaatccgctatcacgtcgtgttgccgttttgattaaccaccgtccgctagcctcacctttcttttaaactgcggcctacgtagtgcaaccaaataaactgccgtactgacccagtgcgcattctgtcagtcgtacaggattacattacacaaccaaagatttctgttgagtgacccaggcgccttaaggcgcccatagggctaatcaacactgaatcaacaaatacctaaaacaatcggcaacgatccaaaatggttgccatgagaattacagagtgcatacatacatgctgtgaatacgcgagtgctatggtttattgcaagcatgtatgttgcaaaaccactctcccatttataatgggccccggggcacttattgatttataaggattttttggcgttttaacatatgtacggggcggttatctggctgtatgtatttacagtgttggttgcgcataaataagcgcaaccatacgcgaattatgcataattaatcaaaaacagtgcataagttaagcgcagtaaatttctgattattgcaaaatgttggttgcaacactttttccttataaatcaataagtgcccccggggcgggcgcggggcgatttttagcgctgtaaactttcgcgttggttgcggttttatttagacgtgcgaaagcaaccaacattatgcacgttggttgcagtatttgttggttgcagcgtagctaatcttctaaatccgagttggttgcacttggctgcggtggtttttcactatccgataattgttgcggcgcgcaatcccaatccttgaatagccacccggtgatctcaccgtcgtcattcgcggctgggatcactacgccggcctcctgggcctcgcgccatccctcttggaacttcttgcggtcgccatgtacctttttgtataccgagcccgccagggcctgcgatataccctcgggcgcttcgtcaagcgtctgtagcgcctcgatcagcttgtacagtgcggatgatgccttgccgttcttctctttgatctcgttggccatagcacgagcgggcgtgacggtgcccgcgtcgaacggcgccagggcgacagggacaaggtacagtgtctcgtctggcggggtggtcttgaacggcgccggctcgaagccggttagctccttggagaaatccggcgctgctgccgtggattggtattggccgacgagctcggattggttctcgtctaccgggatcttgcacgattgcaggatgaacccgcgcggcgattggcgcatgccaaaacgcgctttctcgtggtagaaattgagttggtgctctttatccggatcaggctgctcaaggaagaaagccgcatcaacggcagcatgcagggccccactacctcgcgccgtccggttgccgttgttgttggacttggccgggtggtggatgacgcccggtgatccgcctgtctctctggcgatgtctttcaggcaggccaccactttacccatatcggtagcgttgttctcgtcgaatggctcggcggcgagcgcggtagtggcgttcagcgagtcgaacgtgataatccctactggttcttcccccgccaggtcgttgatcaggcgcacgcacttcttgcgccccgctggggtggtaatgtcgatcccggcgccagcggtgtcgatgatgtgcaggcgggataagtcggactggtattttatctgtagcgcttttttacggcgcttggattcctccggcgcttcggcgtcgaaatagaaacagtgcgccgggatcaccgccttgccggcgaattggataccagccgcgaccgccgccatagttccgaggatgtggaaagatttcccgatgttcgatccgccggccgcgtaccaggtagagcggaagtttagcaacccctcaatgatcgggtcgtgctgcgtgaactgcgggcggctcggctcgtcttccagatcctcatcggtgcagacgtacaagtcctcgtctgctgccgcgtcgttctcggcctggtgcagtcgctgcaactcttcatcatcgattaacggcagcgcttcggcgagttgtgcgcggctgatcggatggggtgacggtagcaggtagtcgggtacgccgagcatacgcatagctaggtgctggtggcggttgatgttaccgtgcccggagcagttggcgtgctggcagtggaaatgcacctcggggtgcttggcattgggtaataggattgccgtggagctggtcccgtccgtgtcggtggagtggttggccgagttcgggcactctacaatgtaccctcggcggctcggcatgagttcgagccccatttcctcgcaccactccatgatcgcacgtccgttctcgtccgctttggcgaggtcgtcttctgatagcgtagggcgatccgatttttcggcgggtgcttcccacgccatggctaaaacgtcatccaccgccagggtgcggccagcgcccgtgcgaaatgccgcgttgcgatgcggtacgaacatcaagcgggcgcgctgataggcggtggcgtcggcgcagtcccacacgtccagcatatgcgccagcgtggtgtttacctgccagatctcatccgcggccatcgggcgatcggtggggatagcaaaacgcacgctgcgggtgtcttcacctttcagcgggtggcgatcacccggggtggtgtattcgagatagcgcaggttgaggcgcgacaatgcgcggcgcaccaggaaaaggcgtgctggtgtgacggcgtccaggtcgagccagcagatagatcggcagtctacgccatcgtcgccgcgcttggcgccaggtagcacagatgcacagatatatttttggcggctcttcttggcccggtattcgtcctcggtctcgctgccggtgaacgtcgggcggttctcgatttcgtcggggtcggtcatgtagtcgacgaatttgcgccacgtcatggtggtgttttttggcctggcatcagtcgcgctcgtcccgatcgcgaactgtaggcggattaattttgcattgtgcattgtgctgtcctatactcgaagacgtgcgatcgttatttctgctttcccacaccaggaacctttaccagtgcggcccgtatcatttttgatacgggtttttttttgtttttaaccctcggccagccagcgcggatcgcagcgcagcacatcggccatacgaaagaggtttttcgagttcatcgactccgcgttgccgttcagtagctgggaaataaaggggcgtgacaagcccgtagcgcgggcaagctgcgccggggtcattgcttgcttggtcatttcggagcggatgcgctcggccagggtggtagtaatcgccatggtaaattcctctatgaatgtaacgtgtgtgcactatagaatacacgccacgccaactttcggcaactcgaacgcaactcaaaaacataacaccatgattgtattaagctttttaataaagttgaaaaaacagcttgcaaacatggtaaattgatttacagttatcaacaccgaaacacggtacacactaaccaactgaggattacacgatgtttgagaaattactcgccctgttcgaacgcctggttatcgcacaagaagccatcgcagccgccggtaagaagtattacacggaagctgaagcggatcagaagatggccgaccatattgaaaaaaaagaggcttccgaaaaaccgaagcgcggtaaaaaagctgccgccgctgaaccggaagatgatccggttgatgacaagccaaaacgcggccgcaaaaagcaatctggccccgatcttggcgcgatgcgtaaagaagtcgaagagctggcccaggtattcgccagcgcggatgatgatgaagcgctggaggagttcaaaaaactcctggaagatttcggcgagcgcaccgtgaagaaaatctctgatgacgacctgccgggcttccacgaggagctgaaaaaactggccgatgagtttttcgagttcgaagaagaataacactacgctggcccggttatcgccgggccactttttagaggtgtaaaaatgagtgcttataattgggccctttgcgatcttttatgccgtagagatttcgtatggccgcgcagccttgcacaaacagtgtcgtcacgcactcgaccgccaaacggttggcgttggagtcttgtcgcgacgggtgatttcattatgcgtgggccaactgacgatttggttatgtacaaagacaactacgatgcggcgttgatgagtttgagaaacaaaaaagatatcaaaaaatacgaaacgtacacctcggctaaaccgtcaaaaagcgccgccgatatcctcacggcggcggcggaccatatggccgagcgcgccacacaacgcgatacacctggaggcgagcgcactatgtgtcgcacggtggccgcattcaacgcgatgtacggcaccaacttaaccgaggtgcagggctggcagtttatggtcctgttgaaaatgtcccgcgcttccgcgggtgcgcatgttgccgatgattacgaagatcagacggcgtattcggcgctagcgggtgaatgcgcaaatcgggaggattagcgatgcattatcaactctatatcggtactgatctgcgtgatggcgcgcaagcgttatggctcctgcgcggtccggtcgatgcgatgaccgagtgcgtggcgttgtcaccgaaagtatccaacgtcgatgtgatcatgaacacgcgccgcgagcgtgatccgtatgagttcatggcaatcgccattttcgaaaagcatgcgcacgccgtggcgccgcttacgtcctgggaggtttaaccgtggccgtcctgaaagcgaaacgcaaaaataaagatcgctccggtagtaacgaggagcacgcgctattgtcgccaagctccgctaaaaagtggctcggctgtcccgcggcgctcaccgctgaaatcgggatccccaacccgtcaaatcctgcggcggaagcgggaaccgcgatgcacgccgttgccgagattatggcgaataatttgatccgcgatggtgaaagcaaggctgcgtctgaattcgtcgggggctacccgctgcataccccgacgaagaaaagcaaggggccgaagttcaccgacgaaatggccaagatggtgcagggctacattgacacctgcgtagcgcccctagtcgatgccggcgccgaagtgtatatcgagtcgcgcgtagaccttagccgcccgctcggcgcacctaacactttcggcaccgcggacttagtggccgtcacagagctgaccgacggatcgaacatgctgatcgtcggcgacttgaaaaccgggcggcacccggtggacgccaaagaaaaccggcagatgatgatctacgcgctcggtttgctgaataaatatcgcttctcgcacgatatcaccaaagtgcgcttgatgatttatcagccgttttgcggtggcgttagttagtgggacacgtcggcggaagtcatcgagacgtttggcaagttcgcgaaagaccgcgccgctaaggccttggcgtgccacgccgccggtaaagccgcgttaaagcctggcgacttccggccatccgccgatgcgtgtcagtggtgccgttttcgcgagaagtgcaacgcagcgcgcaagttcaacgagcagatcgccgctgacgacctacgtgatgagtccggcgacgaaatgacgccagaggagctggccgaggcctacgccaagttaccggcgctgcgccagcacatcaaaaacatcgaatcggcaacgtataaggcgctgttagccggtaccaaactgcccgggctgaaactggtagccggtaaggatggtaatcgcacctggtcagatgaggcgcttgtgcaattgcgtcttgagcaaggcggcgttacgccggatgcgatgtacacgcagaaactgctaacgcctacccaggccgaaaaagcactaccggcgggcgcgtttgagtgggtggaagaactcatcacccgcaagccgggcgagccgtcgatcgcatcggcagacgacaagcgcccggaatacgtgccagttaaagacgacgatttagtcgattaaaaattggttgcaatgtcctacgtgttgtgacctaatacataagccgacgcggcggcccttaccgcgataaaaatgtgaattggagagtgttaaaatggctaaagtcaatctgaaaaatgtccgtctgtgtttcctccacgctttcgagcgcgccgagccgaaaaacaaaggggaaaaggccgcctacaaggtgtgtatcctcctggacaaagacgatcagcaggttgaaaaactggaagacaccgcgttagaggtgttaaccgcaaagtggggcaagcgcgaagttgccgagcgttggatgtcgcgtaactatgcgcaggatagcagcaaggaatgcgccgttaatgatggtgacctgcgcgaagaggttaccccggagtttgaaaacgcgatctatatcaatgcccgcagcccgaagcagccgaagattcaaacgtctttaggcgaggaccagaccgagccgggtatcacggttgatggcgatccgatcgagggcaaagaaatttacgctgggtgttacgctaacgtcagcattgagttgtgggcccaggataatgaacatggtaagggtctgcgcgctgcaatcctcggcttgcgtttccgtgccgatggtgaagcgttcggcggtggcggctcaacggcaaccgatgacgacctgagcgacgatgatgacgagccgcgtagcgtatcccgccgccgcagtcgtgacgacgaagatgacgcaccgcgcggtaagtctcgcaaccgtcgtgatcgcgatgaggatgaagacgatgaaccacgtgagcgccgccgtagcgtatcccgccgccgcagtcgtgacgacgattaataaaaatcctcgatagtacctacggcctcgcatgaggccgtttttctaagggccgcattatgccacaactcctatttcttgacttcgaaacattcagtgaagccgatttgaaaaaagtcggtgcctatgcctacgcagagcacgattcaaccgagatcctgttagcgtcatacgcgtttgatgacggccccgccaaagtgtgggacgctacttgcgcatcaggcgaaagcgatatcgatctagataacaattccgcccccgatgatctgctgcgtggcctgcgtcgtgcaaaacgcgggcgcgtcaaactggtgatgcataacggcttgatgttcgaccgcttgatcatccgcgaatgccttggtctcgatatcccgccggagcacatccacgatacaatggtgcaggcgttccgccacgcgctacccggcagcctggataaactgtgcgaagtgcttaacgtcgatgccgacctggcgaaagacaaagcgggtaaggcgctgatcaagcgattctgcaagcctacaccgaaaaactacaagatccgacgctatgaccgcaacacgcatccggacgaatggaagcaattcaagcactacgcgcgcaacgacatcacggcaatgcgtgagatctactacaaaatgccgtcatggggcgagatagacaaagaaaacgagatcttggcacttgaccagcgcattaacgatcgcgggttttatgtggacactgatttagctaaagccgcgaccgccgcggtggccgctgcgcgcgctgaactgcaggaggccgcgcaagcgacttacggcggcggccttaccggtgccgattttctccccctcctgcgcgatctggcacccgcgcatcacatcccaaacgcgcagaaatcaacgctcggtgacctgctggatgacgccgacttacccgacgaggcccgccaggtgatcgaaatgcggctaggcgcggccagtaccgccagcacgaaatatgcccccctgcttaatggtatgtccgccgacggccgccgccgcgggtgcctgcaatatggcggcgccaaacgcacactccgttgggcgggcaagggctttcagccgcagaacctggcacgcgggtatttcaaagaaaaaccgctagcccgtgggatcgaggcgctgaaacgcggcaccgcggagtacgctttcgacgtaatgaagctggcggcatccacggttcgcggctgcatcatcccggcaccgggtaaaaaattggtcgttgccgactactctaacgtcgagggtcgcggtctggcctggctggcgggggaggattcggcgctcgatactttccgcgcggggttggatatctacaaagtgaccgccggcaagatgttcggcatcagtccggacgacgtggatggctaccgccggcagatcggcaaggcctgcgaattgggtctcggctacggtggcggcgtggccgcgttcctgacattctctaaaaacctcggtctggatctggaggaaatggccgttacgatggctggcactttccctgattaccactggcgcgccgcgctacgcgcctatgaattcatgaagttgcaggaggtgaagcgcaagccgctacccggtaaaaaagacgatcgaacgaccgtcgtcctctctaaaaaagcgtggcttacatgcgattgcatcaaacgtatgtggcgggagtcgcacccaagaacggtgcaattctggtatgacctggaagaagcctgtttgatggctatcgacaatccaggggcgtcgtattgggcgggggccaaggttcgccaagacggcaaacgcgccatacgcatcgagcggacattaacgcggtctggcaagccgggcaactggctaaagatcgaattgccgtccggacgtatcctgtcctatccggggatcggcgtgtcgatggagaaaaccaacgaggacgatccgggcgagaaagcgcgcccacgcatcaaataccgtggagagaaccagttaacgcgtcaatgggggtggcagcacacctacggcgggaaattggcggagaacgtcacccaggcgctgtgccgcgacatcctagcatggtgcatgctgcccgtcgataacgcaggctatgagatcatcctgtcggtacacgatgagctgatcaccgagacgcccgatacggcagaatacaacgttgccgaacttgagcgcctaatgtgcgatttgcctgcctgggccaagggtttcccgctaaaagccgagggctgggaaggataccgctacaagaaatgatgggggctgtatgacgccagaaggtaaagtgcaggcgcacctgcaacgacggtttaaggcgatcggcggcttggtgcgcaagatatcctatgaggggcggcgcggctgccctgacctgtttatcgtgttgccgggtggggtggtggtcatggtggaggttaaaaagcctggcggtacgccggagccacaccaggtgcgcgagatagagcgcttacggcaacgtggtgtgccagtgtatgtaatcgacagtatcgagggtgcggataagttggttgcattttatagctgatttatctatagttggttgcaaggacgcaaccaggagcacgcacaatgcatgacatcttcg

SEQ ID NO: 2 - DNA, 42013 bp, Bacteriophage phi eiAU
catcggtacacgaagccgatcaggttctgcggatggtatctgacgatattacaaccttacgcgatgtgcgccgcgacgagtggcgcaatgcccgtcgcgcgggcacaagccgcgatcgcgaattcattaaatgggatgagaatgtgtcgctagttttccaaaaactttgcgatttgcgcgatagcgcacagtttttcatcgacacccatacgccaccggctattgccgagctttatttccggagaatttaccgtgattagaaaagtgcgccttaaraaacgcatcctgaaaatgtgccgctgctgcggcgtggaaaagccgttgtacgaattccacaaatacaccggcaccacctgccggtcgccagacggacaccgggcgatctgcaaggtgtgtcgcaatgaacaggcccgcgagtatgcgcgccgtaaacgtgcaaagaatggagaataaaaccatggccactattaccaaaaaacaacgcgcagaacttcgcatgaaatttggtggccgctgtgcttattgcgggtgcgaactttcagatagggggtggcacgccgatcatgtagaaccggcattgcgtaagtgggagttcgttaaaaataaaacaagtggagtgctacaaactgcttctacgggggaattttggcgacctgaaaatgatacgctcgaaaacctgttcccatcctgtgctccatgcaatctatttaaggcaacttttagtgtagagatgtttcgagaacagatcgcagaacaggtaaaacgcgcacggtcacgcagcgtaaatttccgcacggcggagcgattcgggcttattaaggttattgatatgccggttgttttctggtttgagcggtatcaggaaggagcagatcaccaaggcgatagtagaaaagctagccgtaattgggaaaggtactcatgatgaatcacaaattattgcgccatcttggctacggtgaattcccggacgcggtcatcgatgccgaactgtgccgagtgatggccgcgaagtacaaaaactcaatccccggtgctctgcgccatttcgcccgagcgcgagccgcaacagtgcgcaatccgtcgctaaaatcggcactggtcaagatgggtgcgagtatctacccggaaaccgggatcgccaccctgcgcgcttgcctggacaagatgcacgccgctgcggtgcgtgaactgcgcgcgcaaggcattacgcccgatgaatatatccgggccgcgggggagcaacatggcacagtttaagcgccgcccgtaccagaaagcgatcacgggccacatcatcgcgcatgctcggtgcaacgtgtgggctacgatgggcagcgggaaaacgggcgcgacgatgtgggcgctagatgccatgtttagcaccggcattctagatgagtcggatcgcgttctgatcctcgccccgttgcgcgttgcgtctggcacttggccggaggaacagcgcaagtggaaatttcccgcgctgcgggttatcgatgccaccggtaacgccgagcaccggatcgaggcactggcaacatcggcgaatgtggtatgcctgaattacgacgtgctggaatggctggtcgagtattacggcaacgattggccgtttactgtcgtagttgccgatgaaagcacgcggttaaaatcgtatcgtagccgcggcggtagcaagcgggcccgcgcattggcgaaagtggcgcataagaaaatccgcaggtttatcaatctgaccggtacgccagcgccgaacggcttaaaggacgtgtgggggcagatgtggtttctcgatgcgggcgagcgccttggcaccagttatcaatcattctcagatcgctggttcgtcagtaagcaagtcggctcgtcaccacttgcgcgccagatatcgccacgcaccggggcggaaaccgagatccaccagaagtgcgcggacctcagcatcacgatcgacgcggcggagtatttcgggtgtgataagccggtagtcgtaccgatcgtagtcgagttgccgaagaaagcgcgcaagatctacgacgatatggaaaacgcgcttttcgctgaattggaaagcggcgaaatcgaggcctcgaacgcggcggcaaaaacggccaagtgtttacagatcgcgggcggcgcctgttacatcacgaccgacgatggcgaggcatccaaagagtggacggaaatccacaaggccaagctcgacgcgctggaatccatcatcgaggagctaaacggcagcccgttgctagtcgcgtaccagtataaacacgacctggtgcgcctgctaaaacgcttcccgcagggccgcgcgatgcgcaaggggttaaagggcaacaatgacatggccgattggaacgccggcaaggtgccgatcatgttcstgcatccagccagcgcgggccatggcctgaacttgcaggacggcgggtgccatctggcctttttcaacgatacgtggaactatgagcaatatgcgcagatcgtcgagcgtatcggccccgtccgccagcaccaagccgggcacccgcgcacggtatacatatacatcattcaggcacgcggaacacttgatgaggttgtcgccctgcggcgcgacgacaaggccgaagtgcaagacctgttaatggactatatgaaacgcaaaaagaggagtaaatgatgacccgcatgctacggtctaatcccgtcgttgccgttcctgtaggcggtggcccggccatctacataccatgcccaaaggccattatgcggcgcggctttttgccagctggcgttagccaggtattgcagggccataaaaaatcgcaccgcgggtatgtgttccgccgggctaccaatcgtgagatcgcggcgttcgattgcgatatcggctatctcgcgccgtcagagttcagccctgagctactggctgcactgctgacgtaccacccgcgcaccggggagatacgtgataagcgcaccggaaagcgcaagggggtctccaccccatccggcggggtaacggtcatcgtaaacgataaaacgatgtgggggccgcgtgtggcatgggtattacacactcggcaacctgtgccggatggcctgacggtgcgctgcatcgacggagggatcggacattatgcacagcgttggaccaatctggaattatgcaaacaggaagatattcgccttgacgaaagcgcgatagacggctacagttaattcgatttaacaaccggtgccgaatatatgactgcctattacaatgaaattgacccctacgcagcacaatggctacgtaatcttatcgcagaagggcatattgcccccggtattgtagatgaacgatcgatcgaggaaataacacccaatgaactcaccgaatttacccagtgccacttcttcgccggaatcggagtatggtcactcgccctgcgccgcgcaggatggccggatgaccgccccgtgtggaccggatcctgcccttgccagcctttcagcgcggcaggcaaaggtgcagggactgctgacgagcggcacttgtggccggccttcttccacctcattagccagtgccgacctggcgttgtctttggtgagcaggtttcaagcaaggacggcctcggctggctcgacattgtacaaactgacttggaaaacgcgggatacgccagcgcagcggcagatttatgcgctgcgggcgtcggtgcgccgcacatccgacagcgattgtactgggtgggtcactccaaccactcgggactggaaagacacaggaacggatatcaaaccgagagcggacgggtcgcaacgattcgaccaattaccgagacaggccaatctctgcgggtggccgacgcctacggcgaacaatggtacgggggccggcacatcaggaagactcggcgggctgaacctacaaacagcgtcattacttgtggggcctatccgacgaacggccactggcgagattctgactggctcggctgccgggatggaatgtgccggccagttgaacccggcacattcccgttggctaatgggattacctccagagtgggacgattgcgcgcctacggtaacgccatctgcgcgccgctcgcagaagagttcatccgggcgtatctcgacacggagaaaaattaaaatgtcgggctatcatgattcaaaaacggcaccagaggataaagattgctggcgcactccaccggaggttttccggtatgcggttcgtacctggggtgctttcgaaatagacgccgcggcggcagatcacaatcatcttgttgccgattactggacgctagcagataacgcgctggtgcaggattggagcggaaaacgtgtatggtgtaatccaccgtatagcgacatcggcccctgggtagagaaagccgctacggcggaattctgcgtaatgctagttcccgctgacacgtcggttaagtggttcgccaccgcgggagaactcggggcgtccgttatttttatcacgcgtggccgtttgcggtttatccataacgcaacgggaaagccggggccgagtaacaaaatggggtcttgctttctggtctttggcggtagtcgaccaggacgggtagatttcgtaacgcgggctggcgtttatcaaatcggcgcacgccgcaaagtgacggttaaaaggcgcgtccgtgcgccgcacaatgcaacataattttaacacaataggccgctgcgtctaccattaaaaaaaaatggttgcaaagttggcggcctacgcctatagttaaatcaacttaacaagaaagaggaatcgcagaaatgaacgacgaactcaataaattgatagatctgattaaggcccgagacgatatcgcatgcaaacttagcgcactacatagcgaaatctgtagccttacggtagaacttaaagccaagaatagggcaattgttgaaaatgcctgcgctttgggtggggattcagtggaagaatatattaacagtgacggtatgagggccttttaacgatgaaacgaatcaccgcaatcgcaatcataaccgccgctatcatcggccagttcgtatgtcggcactgtaggcgccgaggacatggccgcgcatgataagtgcgaatacctggcgtataacggcccgtcggcaccagctagtgcagacgaccgcgacacggcaacgcttctatgccttaacgccgtaacagttgccgaagaaaaccccggcgtatcggttgacgtcctccgcggcattctcagcttgcaaggtgcgatgcagcacaacccggaaaaagaagccgatcaccgctggcgttcgctagccatcctgcacggtttcaacatccaacgcggcaattacaatacgggcggtgcaaaatgacctacttccttgccatgatcgcaatcatgctgacggcagtcactatcggcactatcgacaagaaagagaaagggctgtcggggctatctcgcgggttgctgcgtgtgctagcgatggccgcaatgtgccttagtttcttcgtggcgttcgatatggtggattttaagagcccgtattacgggtacgtaaaggatcagcacaagttaacgacagcgcttgtttttggcctaggtgccatcacgctatcgatcatatcgacgttcggaaagcgtaaataaccaaggggccgctaggcccctttttcggcttcatgcattcccattatcgcaagtgccttaaccgcaatccgctgtaggcgctccatctgtgccgtgttgagcttcccggtagaggccaccgccataatacccgataaactccctagcgccttaaaatcggccatggcggattgtatgatagccgtctgcgggtagccctcgtcgagtatggcctgcgcccgttccgccagattatcggcgacagctttaaccccgtcactcggcgtcatcttcgcttacctcgcctttttcttttcccttttgcggtaccgctgccggttgcggagggaaagggctgatcggctcgcgctgttcttcctgcgcggctggcggtgtgatatctaaaaattgtttgatgaatgacccagcgaaatcgatatagtcgcaagtgtagacgtgcatgcggtcttgatcagcgggcgtcaacatgttggggctcatcatgatcttgtgttggtggaaaccgacatagaggacgttaatcggtgctttgatcgatccgggcggcggaacgataacgcccaaaatccaacaataatgcacctggccatttccgaggctgatcggttcgcaaccgacgttcaaccactgcacctccgagtgcgggaaagttgcagcgatatctgttgcgtccttccgcaaatgccaatctacgccggctatgcggatcacttttttaggcgtcatcccgttgaactgagtcacgcccacgccttggctttggtattggtttgtcattgtgtcggctccaccgtgtttttacatcttgtgcattttttctagtgcggcaatacacgccgcggctagatcaacgagttcgtgctcgataccttcgcgcgacccttgctctttcttggtcatcagctcatggtattccatttctacaacggccatcataccgcccggctgggaaatatgacttgcccaagttgcaggatgttcatgaactcgttcccgtacactctccataaccgcactatgcgaatcatggtgtttcggatgacgataaccagtgacatcttccataacctccgtgccgccggatagcggatgatagtaatgatgatgttctgacaccggcgcgccatggtggcggcgcatccggcgatacattaatactcgcatttttacacctcgctaatcgcccggattgctccgggcgcgggttagacaggcttacgcggatttggtgccgagatgtgccaaaatcgtgttgatgatctggttagtctgggccgcctggctgttttgcaggaagatttccccggacagcttggcgttctgcgctttctggtcgcacaactgagtttgcagatcggtgatgaagttctgctggatcagttggcgagtggcggcgccctccgccgcgatggtcttctgcgtctcgcagcaacagcgggccatatcggcgctgacaccggcgaaaccgctggccacggcaaagcgggtatccgcaccgttgcggtcaacggtggcattaacccctgcgaagccctggcacaatgcggactggatgcccgcctgcccctggaagttcgcatactgagccgcagacagcccattagcgatcgtcatgttggtaccgttctggccttgcagagtttgcagggtgcccgcattcactgcggacgcgacggagttgataccatcgagcacggcggtagtcgcgatagccgcttcaccggcaacgccaccacggttaccccagccgccgccaaagccattgccgaaccaagagccgatcaggccaccaaccgcaccgccaagacctgcggcaccggctttcgccacccatcccgccggcggggatcagagtcatatcagacatgttaacacctcgtttgtgtttgttgaatttttgtaacaactaagttacacaaagagaataccccgcagatcggcgcttgccaatcacgcggggtggtatttttattaaattattgataactattgtattatactcggttcgataccaacaccctataccggcggggtggtgggccattgaatatcgggataactagccggattaatctcccgcaattttttaatatagcccatccattctaccaatttcgccttatcgccatcgcttatgatacctagtgatagctccgtttgccaatcgttcgtggtcttgcgggcctcttccaatagcgtattttttttatccagggcctgagttattaactcttcttgggttggcggtgggttagcaagtgccatcgcctccgtttcagaaataggcattaacccttctttaatgtaatgatcttgggacccatcggcgagataggcgtacaccatatttttagaatctttgaaatatttcatcgcatttcaacccagttaactagcgttcctgtgggacatgatacagaataagttgctccttgaggtattattgcgtaaagataatgccattgtgttagccatgatacgttgggggtattaccgccggccacgacgatatcgcctacttttatcgtagcttctacgctagatggcaccgttacctgtacttttattgcaattggccgggtggatgaatttgtatagataacaccgattgaccggtcggcggtgacatccatatacgcttggcctacgccgatcgttacatcaccggcggcaacagtgccggatgctacgcctacattgagttttgatgcatctcctaatttaagatgcgctataatctcatcggcatttcttttacccagcaattcttcgctaaaaacgcttaaatctgccgtttttgcggaatctttgcccgttaacaccaggatcttattagccgcggcctcaagaattgatatggccgcgattattggtgttttttccgcttttttatttagctcggcatctacataggtcttgtctgcctttttgtctaactcggtatctacgtaggtcttgtctgcctttttgtctaactcggtagtgattttctggaagctgctgcccgtatattgtacgccgtctatttcgaccgtgatatccccggtggcagaatagaactgttttagcgcgtcggattgctcctgataacttcgcaatgtcgcggcgagctggcgggcaaagtccgggcgtgactgcccgtagaagctaagtattgcatacgcagaccctgacggtaagttgctggcgtccgacaccaatgtcatcgacgtgtcgccgtttacacgcgctatctcatagatttttaccacaccggacgacggaacgagaagcgcctgaccctctcctatacccgcggatggatcagcccattttgtacccgaaccggttacctctttgccgtttatggcgattgttcctgttttgtaccatgccattgttatgtattcctatggttagccctctaaaatttcggcactaccgcgtctagcccgccccattaataattgggcaccgtcatatgtgagcagtgtatgcggtttttccggacggattgtattaggccaagtcttggtgaactctattcgtatccatgtgccgttggcaccaggcggaagagtcaaacccgatagattaaatttatattccgccgttwccccgttagaccataattccgtcaagacttttcgcactccggaggattcaacgtaacatgatattctagtcataccgccaytaccggcaaaaaactttacatagccattggatataatggtctgttcaaaatcttcagcggggaccttaaataggactcttgtggcaccattgcccacgcttacatctttacttgaaaatgtaaagtttgagtattgtactacgtcgccaactattctattggcgtgcaccgtccctttaaaatcgccattttccgcgtacacagtgccgcgtatcgtggcgttattgaactctacggccccagatttattaatagtccagccggcgccacccggggtgtagtcatccgattgaaggacgcccgcaattttagcactaccgatcgaggcgtcacgaatgaatgcgtcacgcagatatgcctggtcaccaaccaccccaaacataagttctggtttatctcctactttagccataacggcaaaacgatcggccagcatggcgacttgcgtagagacctcgccctctttcacctcagcggaaattgacatgccagcagaataatattccccgttgtatgttatccccgcgttagtatcccataccgcgtaaccgttgccgtcgtaatcgaacttcgctgtcatttttgttgctacggctgcttcgttttctgccaccgaggctttaaccgcagttaacgattcggccagggcgccaacttcattcaccacgacattatcgattctaacgatctctgcggatagttcgcctaattttttggattttcgttgtatgtcgccgtcattggcgattgcgttctgctcaatggcatctatcgcggcattaatgctcgtctcgagcgccttacctccttcagatgacatgacctgatccccgagcgcatccataatcgaatccgtgtcgattgatgcccgcccctgtacactggcgccccattccgatttattgccgaggcggtcaactaaacgtgcgcgataccaaaagacggcattagctttaagaccactatgggcgaaggacttgctcgggtatggggataaactaagcgttgtaaaggcttcttcagtgtttgtcggtgcgtattggatctcagtgtattcggtgtcctcggcatctttggggaacgcccacgcgacatcaataccaaagactacatcgctagatgctctcagggatactggggcgggtacttcaccctggcggccgtcgatatgcgttaatacactggatgcccacacactagacgccccgaaagacgtgaccgcacgaacgcgcaccagataatcgccggcaaatacaccttgtacctcaaaaccatttacactgctcgccggcacattaacccaatcattggcccctctgcgccattgtgcttcatacgcgacaatatccggctgtactttaccgtcggccatgcgcgacggctcccaggtagcgcgcatcgtcgttacacgctggttttgtcgcacctgctcgtaactggtgatggcgaccgcggtcggtgcgttgacaattccggtcggcaaaaggctaactggtggcttgtctaacctggcgccatcgtctacggcatcatatttcgagtcgttatactccgctgcgctaatctcgaacgtgttattttcgtcgtcgaaatctagcgttaacacgcggaatttttgcaacgccaattcgcctgagtcaactacccaaaccgcatttaccgcgggcgctgccgtgaatggctcggcgatagttacaacgctatccccgaccgaagcaatttttctggactctaccgcgccgccagtggtgcggatcagcaaatcatcgcccggtttaccgtccgtagtacgatcgagcattacctgtttggttttctcgtcatatcccgcgacgcggccacccataacacggcccccgatccgctcgtcggccaaggcgaagaccgtaccgggcagaaaggcgaaaccctccaaccctacgcggagttttaccagccgatctaccgagttggtaagcaccgcccatgatgcgcggcgctgcgcttcgctttcgcgcgtacagccgatcgctgtcagttgggtctgttcgaaaccgagttgcgccacaagctcctggaacataacggccgtcggggtatctgcgtaatggttttgggcgtcactataattaacaagagccgagctaaaacgcgttttacggctgccgcttgaataggttggcttgccgatgatagaggcgcgggtgacgatctgcgatggtgctttcaccggcatatcggaaaccacattgaacatgttgtttccccaatacgtcagcccgttaaatcccgccgcgatatcacggatcaccgtccaggcatcggcctgcgcctgcaaataacagttgaactcgaaacgcggctcaacaccaccggcaccatccggcaccatctgatcgcagcgctgtgcgatgcggtacaactcccatttatctagcatggccgccgtaacgcgcttaccaagcccgaaccgcggttgagtcaatacgtcgtaccatacccacgccggattgttggtgtaggcccatttaaacgacccatcccaattgccggaataggtgcgggtgtctgggtcatagttacttggtactcggataatacgccctttggccaggcacgatattttcgggatattctggaatgattttgcgttgaactcgataaacaacaaggcggtatgcgggtatcgaaaacgtgcgtcgataacctccgtgatggcctgaattttaagcgtgttggcgagacgtgcgctatctgaatccggggtttcacgcacaacgcgaatccgccagccggacgagctacgcggcaggttgacacgtatcgaccgctcatagagcgtagttgttttacctgagatggcgaatttaccctgtgtactaaacgtgcctcccccaacggcaagatcaatccggaacgataccgaagtgccgactacgtcgccgtcatcctcctggtacattagcgcgggaacgcctacgcgaacgacgacagcgtcgatctcggttctggttagcgcatgcacccacgggctggcctgtttaatttcagtaccgaagcccgtctcgttttctactgcggggaaacccgagatcgggtcctgctgctgtacgccagggcggacttcccaacgcacgccgtcgaagttacgcgacccgtccggattgcctagcggcgttccgtccaggaaaattttagtgtcatccaaccccagcgccatttccccctcgcctaatgcgatgagtagacgggcctttgcctggctacgaatactatccggttgctctaccggtgtatgcgcatcgccgccaccaccctttgcgcctttgatgtaaaaaagttgtctctgcattatgctacgtcctcggcaacaattccggcactgattatagcaccgccgatctctctcgtcccccatagcactccgatcgggttacccattgccgtggtgttaacagggccgccgaaagcgtaggatggcctattatccggatcctctcgcgattgtaatccgcgcggctgcggggatagcatctggtaaaggcccccagccataacggccgcacccgcccaagccatttgcgcaccgaatgacgccaacgcaccacctgagaaaatcgtaagacccacgcccgccacgacaagaacggcgccgagaatggtctgaaatagaccgccttttttcgatccctcgatcaccggggcgatgcggatctcatcgttgcctatgccatcgtgcaattcttccgcgccgatgtttttacgtccgcgaaatacggcgaacgtcatrccgttctttttggcgtgcaagagatactcctcgaatccatcgagcgttacgcacagcgcctttaccgcttctgtcgtggtcgacaccgcgcgacgatgcacccggccaaatgccgcgcccagtttgccgtacaggcggattgtgattaatctctccacggttttaattcctccggtaaatctcgatgacggatgcataacacggtgcggtccacataatacccgccatagggtatctcctccgacgcgcggccatataggtggtgcaaaagcccacactcggtaagtacaccggcatgattcggaacatcggcccgcacctgcatgatgacgacgcagccgggcgcaggatccgcctcgacgaaacccgctgcggcccaattatccatgtataggttttcgccgcgctcccaccaagggtaatttacacggaaatctaataggtcgatgccttgccttttatgccaggccatgactagcccgtaacaatcatcggcgcccaacacgaacggccggccgattagcggcggatcctgcggtgagatctcgcggtactcgtcgcaatctggcgcatataccccccatatgattccgcttgcattacacgcggccttatcagcatctgatggctctgccgtcgccccgtcgcccgggtgactgtgtatgacgcgcacaatgtcgccctcgtcggcagccgccgcaaagtcagccgcagagatccggaaatgttctgtcggcatttcatgcgtattggtaactctgatatatcgcagcgcgcggccattttgtaccactaggccgcagcattcgttatagccagactcggcggcgtgttttttgatctcgcctagaataattttgttcatggcatcaccggttgatcagttgggctgcaaggaatccgccaaaatccagtgctgcggccttggggtcggccatatcggccccgaagcgcttaacgcaatcggagtaacacccaccacagcgatcaagcgccgggtctgaaacagggttgcccttagcgtcgaaatacgcgttgccgttgtaggtacagccgtcgccgctgcggtactgtccgcgcatcgcccaggtgcatagcgatgtgatctgacgtgtcggaatttttagcccttgtaaatccgccggactgcttaacgtccaggtgatcgactcgtcatcctcggctgatttagtatcgacccaaaaagtttgataactgcaagcgtccggatcggcattggggttaccgtcggggaagttgcgcgcgtcaaggtattcggcgtaggtgtaaatcacacgtactttcgcgtttatcaagtcgcgataatccagacataagcggctcaaatacccggccaagttcgaaaccgtgatcgtaggtgtcgcggcctgttctgtagacagcgacaacccggacacgctaaacggccagaagtcgaacattaacccgccaaaatatatcggcttcggccctaacttagcctcgtcgccggccgccgcttcgatctcggcagcggagtgcgggaagggggcataatgcatgcggtgtacgccggccccgaactcggaggcatccacctcgactaaaacgatacgcccggacgggtctagttttgccgattggtctatgtacgacgtcatgcgaacaccccatacgcccggttcagcgtgaaggtaacctcggcggtatgccccgttaggttaaacgccaccgattcagcatcgacgcgatacagccccttttcttcgccgggcggagtgaagataaatgccttgacgacgtgccgtaagaggaatgcccgcacctcccgcattcgagcgttgtcaccagcgcaacgcaaggctatggtatcggcgacggagttgataccgttttcggctacctgttcgtagccgtcgcccatcttggccgagcgtaccgattttttataggtgatcggcgcggtcaattcgcataattcagtaaaagtttctacggtcatctaaaaatgcctccgctatttctggaggcattttaacaaacgttggttgcrctgtckaatcacgccatccgcgatttgtatagcaacccgcccggctgtagcatacgcgtaacttctgccgttacggtatccttcatctgcttggcgatcgacgcgccaagggcaccgccggttgacgttgctcggcccattgcaccaccatcagaaaccgtaaccgagatgttattaatgatcgatgaacccgttccaccttctgccgatatgcctaaacgtccatcggcggtgcgcttcaacggcataatggcctcaggtccggcttctcccatcaacccggcgcccttggcgaacttcgggacggcgtcgaaattaaacatggttggctggtttaccacctgcccagaataccgagaaagatcaccaccggaataaacgccgcctttcgcgttagcgaacaacccgcctagccccaatgcattagcagccgatttgattccttgcatcactaatagttgcgttatgatatcggcgatcatctttagcacgtcggcggtgaacgacttaaaatccattttgccggaagtcacaaaatccgagacggctttgacaccgcgattcattgccgattctaccgcttgccccgcgatatccgcgtaattcatgctggcttcggcccaatccttaagccctttgatcgccccagcttgccagtttttatccgcggcgtcctgctcggcccataatttgcgctttgcctctagcgccttctcgatatccgccgttgtggcgccctgagcggccagtgtagcacgcgttaccgcttcatcgttgaaccgcttcgcctctttggtgctcatgccgtaggtggccgctagggcctgcatttgggcgtccgtctcggcaacggcttttgacacattatcatgagcctcagcctggcgctgcagcacaagcagttgatcgccggcatccgccagttgcaccttagacgcccggatctgctcatagctcgccatgatttgtttttcggcaagtgtcagttgccgtttttgcgaggcttccaccaacacgctatgctcggcctcaaatagcaggagagcgcggcgctgctggctggcgttgcggtcatacgtgtcacgctgcttcatcaatgcgatctgcgcttccagcgcgcgtagttgggcggctgactgctcgtcaacacggacgccggcatcgactttaacggccttggcgcgtacaggcttttcctcatactttttgcggatctcctcaacccgacgattgtactcggccaggctgatcaggttggcatctagcaggcgcttctgtgtgtcgatctcacggttttgtttctgcgtgttagtggcgaacttctctgcctccgatgccgccgtcgcgttgagcacgttttgtttttgctgctccagcgcggaggctttgatcgccgcagcttttttatcctcggcctgtgtctgctgccgtagcgtttccacctggcgccgggcagtctccatctggtcatatagcgcggcctttccggcctccgttactaccttgctatctttttcaagctcccagcgctttttatacgccgtgtataccttctcggcagcggccaattgcacaccgaccgattccgggcgcccgatatctaagatagcgtcccacatattcttggcggcgttacggatcgacatcatagcgagctctaccatgcctaactgcccggtaacattagcggcaagatcagtaaatgacgctgaggcgatgcggttggcctccgcgatggcccgcgtttgctgcccctcgtcgatcagcttctgaacacggataatctgctcttcggtcaccgccttgtattgcgtctgcaaggcgcgtaaaccgccgaccgggtccgttgacagtttggcaacttgcccgattacatcgtccagcgattgccccgacgctttggcgaacgcctgaactgatttgcccagtgcgctaaaatccgcgttggcggatacaccagcggcggcgagcttctgcactgccgatactgtgccacggaaagacccgccggagcgttcggcggattcccctaatttaagaatttcattagcagttaggccggagaaatggctcgtcatattcagcgtcttattgagacttgagatctgacgatcagcattcgtcaccccggcaccgatagtggcaagggtcgcacctacggcggcgatggataaccccaccgggccgattgtggacgctagggcgcgcagagcattgccgatgccgccaaacatatcgcgcaattggccgccctgctggataagtaccgttaacgggcgctggccaccctgcaaggatgttacgatgtccgtgatctgcgccggcacgccgcgcatggtggcagtgagctgcttctggctcatgccgaatttgtcggccagttctatctgccgttgctgcgccttagccgcgcgctcggtggcggcggaatttttctctaatgcggtcgccgccgatccagctgctgcgcctgcgccagccactccggacccagcctttttcgcggcgtcgcctaacttatcattagcccgttcggctttttccgccgctacggtaagtttgtctagctcggtgcttgctttcgatacgctgccgacgtcggcgcgcaaactgatgcttgcgaatctcttgggtcatgatccgccctcccgtgatgttgccgatattgtacccgcttatcagggcgcatgcatgctcggttatttgcataactgcgtatgcgcttgcgcttcgtgtaatcgagtagaggacgaggggtggctgatcacccccgtctcctcttacgaagtaccggggcgattttggggcgggcggcttttttctttttaaatcaataactgccccaggggcgggcgctcggggcgatttgttcggggcgagttaaacgggcgttattttattgttaaattcggtgttggttgcattgttggttgcgcttatttattcaaatggttgccataattattcatactataaaattggttgcattgttggttgcacgttgctatagttaattcaacttaacaggaggagcagaaatgattaaatctaaccacgttgacgcctatacaccggcggcgcaakcactattggcgactcgttcgaaagagtggctaaatcggtcatacccgcgtaacggagaggcgatcccattctatccttacggcagcccggtttatcagttcaaccagttattgcggggggtgcgtcggtgatcttcgataccctagaagatgcgatcgattacgccaacacccggcgcgcattatccttcggcgcgtcacatggggcccgccactactgtgtgtatctcgacacaagggaaaatatgcatgtcgcatcccatgttgatctgccttacatcaagcactttgatcgcgtgatatggtccacccgtgcagaagtaggtgctactgggtcacgggggaggccacgggcgctcacacggcgtcaggaagcggaattgcgtcgtatgcgtaatgatgggttgccgtatgctaagctcggcgcttatttcggcgtaactgatatgacggcgtttaggatttgtaacagggggaattaattatgggctctaaatttacaacacatgtcgatctactcaatgtggcgctcaatgaacggtaccaccgtatccgtaagagcgttaaacagccgtatggtcgggcgcactggtataagcggtatcgtcgcgaccgagttattatgcgtattttaaagcttcaacaacgtgctattgttcgaagattaggcaagaatgtcgagtcacttgacatctaacaatggctgtaaaacaaaaagcggggtcgctaccccgcttttttcatttcctctttcgccacttcctcaagaacgcggatgtcgtctagcgcctggcgctggtcggtgatgccgtgaatgtcgaacatccaccgcaggacgccatagtcgagggcgtaagccccgcacggcccaacgcgccattgcccggccatcttggtgaatatcgacaccaccggccatacgtccggccacacttctacggtcttgattaactggtccggcgaaacgccatatagctcttgcgcaatgtcttcactcggcgggacggcatacagcgccgccgccgcctctcttagtttttttcgcggaggcactgcaacgttgtagtgtattccttggtgatctccccgtacgcccgcgggaagttcttcaccaatagcaatgcgttttctttattcagcggatcaccaccatcgacgcgccagtcggccagaaggtatagcaggctctcagccatgacgtcgtaggcgtccttatccgggtcttgtaatgcttcgtaggtgtcggccatattacgctgatattcatcaaccggataatgccggaaaaccacatctaacggcacgggcttatcctgtcccgcgattggcaccatgaccgtggcggggaacgtcggcgcggggtttaacttaaacggtgatttactcattgtgcgaagtcctcttaataaaaagccgccatgcggcggccttagtatggcacggttgccgttacagtgcatccacttcggatttaatatagatgcgcataccggattgcatcgatagcgctactgacaccgtttcgacgttgttaacttccgatgacgggatcggctggaatgacaccttagcggcatacaagcggatctcgcctttaccgccagccgccgccgggttgatgaacttgatcgccgcaaaagcctgtgtctggtctgctttttccagcaccgggcggattgggtcctcaatatcatgggttagcgtgtaggtattaacgatcgggtttttgaacgtgttcaggttgatcgcctgcttcgattgcagcggctgaaatgaaaccgtctgctggtcgccgccgctggtagctacgttcgtgatatacgggaaatcgacgaagcccgagatcttgaccatttcccccggtaccgacggagaaaaggcggacgccggataataagtggtatccgtggtgtcgagttttagcatggtgatcgttttctgcgccgtgtctacggccttcacgatcccgcacaggttgagcgcctgagtccacggcgatttagtaagcatcaccgtatcgccttttttcaccgccgcgcccgctacggagcttgattcatcgtaggtgaaaacacattcagccgcattggtcgccccaatcactttgatcgggtccgacagcgtagcgcccatctgcacactactgccgtttggtaattgataccccatgttatgatctcctcatagcccccacggggcgattaagttaacgctctgtaggttacagagatagggatattataccccgtgtctgtcgccagtccattaaacaccgaagccgggcccgaaacaccaacggcaaaccccgaggcgtcgcgcagcatcagtgttgccgggaatgccgcggcgatgtcgtctgcaagtttttcaggtgcgcgtgttccatggcccgccggaatcatcgcggttatttggaaaataccctgatagatcacatcggtttgctgtagcgatatcgaatcactctctgctggcatgaggttcggcactaacataacattggtcagcgagggccgatccgtcggggtgttttcccacgctaccagaatatcgccgagggggtatttcaaccccgccactacggctgatagatggccctctagcagggcgcgtatacgggatacgctcattgtttagattctcctattgcctgtgcaatataacggcgtgcttccgcagcggttatgcgcacgatcccgttaggggcttgattagaccaaccgaactccaggcgacgcgcatacggcacattattgcaaaaccaaacgctatgcacttggtccaagtttagcccagcaagaacggcatcacccgccgccagtgtagccccgcccgatttatcgatgcggtcgattgccgtatctatcggggcgttaaacgacacctgccagttacccctgaatcggccgcctgtatatccgcgtcccacttcgcgccgctgaatgaaggccaccgttttaccgttgcgcgtcttgaattctctgcgcactccggcatgcaccttttgaccgcgctttaatcgccccgtttttgtaactctacgcggatctttacgccgcatagcgttaatccggctggcgcgtgctttagacttagacagttcggcattaactttccatcgcgaggggtcgccgaccggggataacgtaatcaggcggcccagtatcttcatgccgaaggcccgcactacttgatccatgcgctggtttgtcttatccgcgaaaagccgtaccgactccgaaaacgcgcccactgttaccccctaagctgcaaattatatgcgatcactgtcgagccattcggcgctgccggattgggggtaatcacgcggtactgtttcccgagaatggttaccaggtcgcctgtttttacttccgcaccggcaccagacgcggcaaaacgcacatcgcccgccataatgcgggtgccgtctatctccatcggcttgtattgcgccaacacgccggtaatggtgaacgtttccgcagggtggataatctcatctaccccgacgcgctccacccacgccgggcgagttacggttgcggccatgccgttttgtgataacagccgatccgccgtcttttgcagtttacggtaattcaaagccatcatcgttttccccgacgttttgcgcgagctctacggcggctgtttgtggtacaggctcggccggagccgatggcggcgtctgttgctctaccggttgcaccgcaattagcgctgcagttactggaatgctaatcatgatcaccccctcgacacgctaaagccaaaggccatgccgtcggattccgtccacgacccgatcaacccatcaagccacggaatgtccgcgcgcatgccgatcgtgtccttgtcgtaggtcacggataccgcattggccgccgacacactgatctcctgtgccccgctaatgctcggcataaggtcgaacccgtcggcgcataatagcgccagccgataaaccgccgtaaacacgtcctgtggtgttcctgtcacggtgcgcgggtccacaccttccggaacgtcgatcggcgtctctgtggcgtccagcagcgcacaatcaccccacgcgatgccgatacgtggccaggcgtcggcctgatatttgtccgctttcttaccggaccattttttcgagttgatgaaatccgtagcctttgtcagcgcgatcgctgcgtcagcggtagtgatcgtcatcccgcgttccgcggcgaacgctaccagtttttcaggtttacccaacatacaacccccaatacgaaaaaggcggggcaaaagccccgcccctagtccgacatagtgccgggttagaccgccgcacccgcagtggtggcggtaagtttcatcagtacgccggcggtttctttcaggttcttaaccgggactttttgcgagccaccagtgtttttaaccggtgcgttatctactgcgccctggtccagttcccagtttttgtaggatgacacatccgccaatttagcggagcgcaagccctcgatttccgtgcgcagggaggatttgatgcgatatcccttaacggcaacgttgaaatcaaactcgccctgccaccagcgctcgatgttttcgttgccgcctttctgttctgccagcatgtcgaggccagtagtagtgatcgcaaccgcgccgggtaccagaccgataatcgcatctgggccgagtttggtcgaggtgctggacgcgataacatcggcaagcgcagtgccaaccgcatcagagatcaggaagcgacggccaaggccatcttgcagcacttcaatgttgccgatcgcgaagactttctctgcagacggtacggcctgataggcgataaattgcgcccattgggtaccggacatcgcccaggtcttgatgttgccagcggcgtcgccgaacaggctagcggctaacgggaagtcggctagggtcgggaacttcatgccggtagcggtaacgttaacgcgcgccggttgggtgtactgcgatgccgcgttactgcacagcgcgccaccgacggcaccgacggcacccttgatgtaatgctggatgatcgcctccgtcgccagtgcggatacttcaccggcggtctggttaacgtcggtttggatcttagccatcatgccggacgtaatagcaaccgggccgactttcgcagacaggttaatgctgttggtcagcatgcgggccagcactttggcatcggcggcagtgcctacaggcgcataggcgttacgatcggacaccaggccatcgataatgccaacggtgactttttcgatcacatctttcagcacggtaccgctacccatgacgatagcgccgccgctcgacgcgttccacacgttcagattatcggcaaccaattgaatggtagtgccaattaatttttcctgaaatacaggtaagctcatctttatggccctttaatttatgggccgtgcggcccggttgtaaccaaatgtcaaacacataataccacaggtcgcacgacggccaaattattcgccgataccactagcgatctcggtcgcgcgttgcgccaaggtactctgcggcatagcgccgccgccagcaggctgaaagccacctttaggcgtgccgcccgcgcctgaggcaacaacaaccgcggcataatcggcgttagtgcggaattctttttcgaggtcatccagtgacatcgccgacggtttgccgtctttcatcacgcgcactttaaaatcgccgtccacttcttccaagctcaggcgcggtgcaacatgcgggagcatcaacccggcatttttaccgaaaacgcgggaggccagatctcgcgcagtggcgccgattgtcagatcctgcactttcttactcaacaattcggtgcgtccgctggcctcggcttgcactttagccagtttttcctgccaggatttatccagcgcctctacgtcaccagccttgcgcgcggcttcttctcgcgcttgtttctcggcttcttctgcggcgcggcgcttctcggcttcggttttcttttcattcagtagcgcggctacctgtgattttaaacccgatacgtcttccaggccctcgacggccagtacgtaatcgtcgccggacttgttgtaaagccctttaacggcatcgtccagcgcgtcgaacgctgcggaatcgatcttaaacttcaacataactttgccccctaggcataagttaaggcagggccaccccgccttttcatcatggaatactatcgcctcaattggttgcgtgcaacttagtcacgccacaggcccttatttttgagctgcgctatggtgtacaactcgccgctatcgttgaacatcttcggtaccttaagcccgcgcagaatctgatctgcgcgctcgatgccatatatttcttcgaggacgtgccgcggctgccgttgtacccactcgaaataggttgtgccctcggacaccgattccgacatatagcgcccggtcgcgggatccgtctttagcgcagggcgtgttgaatcaggccaatcatccaagccgcgaataacccatgtctcggtactccggcagcagtaatgcaatttacccggcccggcgccgtactgactgcccgcaatatgctttttcagttcacgatctgccgatccttcggtgtccgctttcacttttagcgggtagaacagccgatcgcgtaattggcacatcggcgatgtatgcgtgtcgagagtggataaccaccgccggcacttcactacatcggcattggcagacaccatcaactcgcgcgcggttgcgctatagtgattcacggcagatttcaccaccgaagacacgccagatgagaatttaccctgccaggtggcttttactcccgcgacgatctccatagtgggtttacccatcaaatagcccgcgcgcacctgattggtgatcaagcgcttcgcccaggccgccaggtcatcaggccatgacagtagcgtattaccctggaacggcgcagagaacgccgccgcggctacctgcgcgcctgtaatggccgcgatgggtactaccgcacctggtatcaacgcggggcgaattgccgttgttagcgcatccgcgtaaaacgatgcctcggtgtcggcgaactcttccatggatgtggccagctcggcgaaatcatcgcgcaaagcctcgctaatcgtgctgtctacctttcgcagggcgcggcgtaactgcgccggggtgattactgcgcctctcaccttctccgacgcaagggccgcggccagcacttcggcaacacgcggcgcgagccgtttaatcgcgcgcagcactttagccgcttggccattcgagaaacgctggacgaaaatatgccggcggatcagcagatccgccagcttatcactggccttcatcattcacgcccccggtcggcggttcattctgcacgcgtagcgcctccatgatatcttctacgctgcgcgaatcatcctcaataccctgacgcatcagatagcgcacaaatgattccaacgtaatgacaccagtttgcacaccggccatcagcgcggtgatcgcctgcgggtcgatatttagcgctgtataggtgcggtctaacatcacggagccctcaccgacaacaaaacgcccggcgatctccagcgctttgttaaatgccgcctctacgttgcccgccgccagggctaaggcagaattatctgtccgggcatcgtaatcggcctcagttgccgttttaggggcggatccgcgctcaatcaaggcggcaccgatcatcgccatctgtttttcgcggcgctcgcacagggttaaacatacgtttcgatcttcggcctgcaacaacgaggccgcgctatcctgcggcagtggtaggccgcgagtagcccctagtgcgatgccgttttgcaggtttttatctacccaagtctgcgtaagtccggagactactaacgttggctgcccgactacatgcgccagctcggctaaatcggcctcggcggcgaaatgcttgatgttcagccccgccaaatctgccagcggcgccgggtcaacactggcgttattgtcgaatgccccgccccatgcccagggcaactcctcgagaggcgatccatcacggtcgcgcaacggcacaaggtcggttttagtgaagccggacggataggcgccaatggttgcaccggtgttgtgaatccagcggcggcaatgtgccacgccctcgacgagacgcaactcgacatactcggtgacgctgtgcatagcgaaatcatcgacatcatcgggcatcacctctacggtgcgttgtgttacgacgagcgtagtccgcccattctcctcgcgccagttgatcacctcgcgggccgaatacaggtcgatcaacacgcgttgccccgccgcctcttcggcggtcatcggtaccggattaccctccgcgtcgtattttgccggtctgctgaaatctaccaggaagccaaagcggccgcctttcagcgcggcggatagtgccccgcgcaacacctgagcaatcggcagccctctgccgtccgcgttctctcgcagcacatccagcgcaccagacaggctaacctctaccggcttggcgaacgcgacgcccaatagcgcttgcagcgtgcgcccggtagcgttgaggaacggcgcacgggctagataactgtcgtagcgcttcgacgccatcgggtcttgactcgggtcaaacgccggatgcggcaggtacttcgtcttctgtgccttaacggcgcgctctcccgctacgcaatcgtcgatccgcgcccactccggcgcgtatcgcgcataatccgggtgtttggtatccacaccagccataatgataatcctcagtagaaattaacgacgatcgaatcgttggccgcagggcgaacaatcggatgacggaatgcaatcgggtagccgatggcgtcggccatatggtcaacacccgacgttttatccggcgcgcctgttttcgggtcccagatttgctgctctagtgcctcggtagccttggggcatcgagccacgttcacttttaaacggcgttggcctttgccgtttaggatcatgccgtttacgtcattgacgcgatcgcgcaccgccgggttgacgctattgtatgacaccgtaaagcctgcgtcctcaagcatcgcaatatcggacgtattggcgttcgttgtcttgcggttctttccgctggagtccgggaaaatctcaattaacccgcgcgcatggtgatccgggtatcgctctttaatcgcgtcgatcatggcgtcggtatccagtaagccgcaaaactcgtctaccaggtgcatttcctcgacgccatctgcgatccgctggacgtacaccgcgccggccatctgcccgacgttaaagtccatgccgatcattaaggtatcgtcatcctcggcaacggtatcacagttattcagcgtgcggctgaaattgcggtatacagcgccgccggtcaggttgacgaattcaccgtttaggtaagcctcgatgagctcaggtggatattgcgcccgcagcgtgtcgatgtagtcctgcggcaaatagtgattatcagtggtgcgggccttgatcaggcgcttctggtcgtccgcttccgatatgaagatctgatacatcgcccggaatccttccggagtggacacgatcaccatctggcggacattaccagtacgcaaacgccccagcagtttgcggtatgcctccatcgcaatatctggcttggtagtatcgaactcgtcgcacacgcaccaggcggcgttaacaccgatcaggcgggtgtagttctccatcgagtcgcagataatgcgcgtcatctgccccgcaatacggcaatggtagatcttatcctgcttgttgaacttccattttatcccagcttcgtttaacgccttctccagctccggatacatgatcttgacgagtagcggtattgtcggctcggtgataatgccatcgcaacctggattcaggatcgcgagttgtaccgccttgcgtgctgccgaccatgttttaccgccaccgaagcccgagcacaggcctagtattttggtcgtcgtatcgttgatcaggccgcgctggtgcggcagcgtcttgatccgccatgtgcggcttttccgctcctttttcaccggcgcggggacaccaaggcgcattacctgccgctcggctttagcaatacgcgccagcaattcgcggtcgaagtcatccatcacgcgttaccgtcagcttctgcgaccacattcacatcggaatcatcctcatcgctaaccgtatcaggttgctgatccgtgatgccgtggtttgctctcatctggaagatcgcccaggcggacggcatcagacccaccccggcggcctggctgatgaattcctgttgcagttctttgcagatggcgtaggcctctgcgaagtccggattagcgcgcgcccataacatcaggttccagcgcgtcaccccgatcaggcgagcgaattttacgaaggacggctggttatcgcgggggatcacctgggcattgcccttgtcggtgtagttgagctgccaggcggtcgcattctcgaagtattgcaccattttctcggcgtactccgggcggtagttggtagggcggccgtacacgtaaccttccggcatttcaccatgacggcgttttgtaggcatgcgcaccccgtttactttaggcttgagctttgcttttagtgccataaggcccccatgttgctgctctgcggtggaccatccaaacgcgagaggcggacccctggcccgccccttgattatatagcgcgtagaaaatggcgcaatgtagcaaaagtcattgcaacgcagcagttaaagtgctttaatacccttacacaaaagctctttcccataagccgtaggtgttccaccaccgaaagctacaaaaaggcccgcattatgcgggccttttcttttgcgtcatccagggtacacatcccggcgtaattcgaagtgcgggccatcgtaaaagcgctcgtcatcgctgcgcccgttctgattccagtcgccaccccagcggatcgcaacgccctgctctttggccgccgcaaacatcgcatcggacactgacttaaatggtgcgcggtcgttccacgggatagcaccggcaaccaacggcgcgcagtctaccgcatgcccggttagatggcggctgttcatcgtctgcgatttgccagtagcaaccatttcgcgctgccgttctacggaccgtcgcccctcgatcactgtgaaatcgaccggtgtcagttcgatcgcgcgcttcactacgcggactaggtcagggtgtacgccgtccagccgtgacaggctgcgactgcttagtttgaacattattcctccacgggatcataggtaatatggaaaatatccggtttgcacggataaaattcacccttcacgcccttgataatgtaatccccctctgttgctacgtgacgtgcgcgcttatccggcccatcctcaagggttactacatcacaaaatgcaacggcgcccgggtgtctttgtttgcgaatgtttttaaccgcgcctccgcaccactctaaaaatcccgcggatggcggataagtaaaacgcrtagcttctatwttaactggctttttcacaaataacgtcggcattttaataccctctaaagttaattcgatttaatactagcgcatttcttgctgtttgtcatagctcaattcgcagagattggccgcttcccaggatcgatcggcgaaagccgccagctcttcatttcgtcggacagatttttcgagcacgtcggtaagcaatacggcggtttttccggtgctcgagccagcgggctgagagtcggaatactgcaagatggtgcgacgtatcgcggcgatttctgcgcgcagcccatcagcagtacgacgggcagcgtcagcatcggcaacagcctgattgcgttctgccagggcccgcgcttcgtcttcagcctttgcggcgtggcgtcggtcatcttccgctttgcgcttcgccagggcttgtaggtcccgctggttctgctctgctcgatatcgttcatagccgttcgtatccccccgctggtatagccaatagccggttgccgttaagccccccgctagggccagcccgctcaataacgcgatgatggttttattcattgcgcaccttgcccttggccctgctcacgatgcccgcccactttccgcaggcgggcttccagaatgcatcccggataagctcccgcgccatcatgaacgcgctgcacaccagcaatgtggtaattagccgttctggtagttcgcctgtctgagagtgcagcaggccgccgagactggtcacggcagccgccatgtacagaatacgcccgattacgccgtcgcgcacttttgggctgtagatgccccatagcgccatgaggaagatggccaatagcgccaccgagtagatcacgttgtgctcgatcatgattattgcccccgcttaaaggccccgattaggtccttccatcttgcggcggcgatcgcgccaatgtccagcgaatttaacgtttctttaacacgctgcaaaaggctcatgccgaacacgccgatgagaaagccgatagtggcgatactgcgctcgtcagtcagtgccagatagtggccaaccggctcgctgaggtagaacgcggtaaccacaccgccgaggaggaaacatgcgcgctgccggaaggtgmcgatctcggtatgcaccaccaaccccacgattgcgcccataacgcccgccaggagcacccaagacgctgatgatactttctgccataagtccataagaaaaggccccgtaatgtgaatacagggccatggtaacattcgatgcgttatcgcgcgattttagcgtttaaacgcaccgccgatgtgctgaaacagttcatcgattttatcgttgatgcgatagtcacggctaagcttcttagcatcgagggtgccgttgataccaattggttctacgccggctttcgcagcgcggcgatccgcgcgttccgtaatctcggcaacgatatcgacggtaaaagcgtgcgggccggacaggctttcgtattggcgcgttgccgtctctacctcggccatcgcgttattcagacggtaaccggctttcgcgtactgcgccaggcgggcgacggctgcgccatagaaatcatctgttaaatcacgtccgtgatcaccaaaaactttgcagtaatggtgatggacatcccgcgcgagccgctcatacttcccatcagatgctgcgagcagttcgcgggtctcctccagcgcgcgcagtcctagatcgacgggcatagctatgcacggctcgcccgtagcgttagagaaaacccccgacagccgatcggcctcgcgctgtttcagctctgcgatcagcgagtcggcagcggccaggcgggtgcgaagcgcctcgttttcggcgtgtagttctttcgtggttgtaacagccttatccaggtcaatgaatagcgccgattcaaattcgttcattatctgctctccagtatgtaaattgcggtgtggcgttatgccggtcaccgcgttgagtatagtcaatttaacaatcttcgcaaggcatgttgccgatccgcagtaattcgtcgatcgtcgcctgcwtttkggctagactcctcttcgagttgccgattgcgctctgccagctggtggcgctcgcggtcgcagcggttgaacatggactgtaaacgggcgtattgcgggttcattgcaatctcccgtccatcaattccagttcgcagcgcatagagtcgagctgccgctcaaggcggcggcgttcggcgttatacatcttggcacgcggcagggtgcgccactggtagtccgccttttcgattgccgattgtaagaatgcgcggcgctttgcctcgcggcggtacttcatgatggcccgcataggagccgtgatatacgcgataagtttttgcatggcgctactccttcggcaatctgatcatcgcgttaaggtcttcgcgcgataaggctagtcgcgtgccagttgctgcggcttgcttctcgggcagcaggtctttcgcttcgggccaggcctcgaggagcttctcgacggtgcggaatttgctgatggttgctttaaccgaggccttgatctccgtatagcgattgcgcagtgccttatcctcggcctggaatacggagaattcatccagtgccgttgtatccgcgattgggtaccacccggagttagggcaaaacgcttcctcatcgaagtcaaccatgctcccctcatagagtggatgggcttcaccgcgcccattgcgataaagatacatccgcaggccattaaggttaacttgtacgtagcctacttgataaacgacaaaatatgcgttctggttagtgaaacacttcggtaaacccagctcctctatatgcttcaccaaggcccgcagcgcctcatcagacgttttgcattccgctagggcgcgctgccgggtacgttcggtgaagtcggcgcggcgggcgcacagatcgcgttcttgttcgggcaggccagcctgcaccagtgcgttagctacgactcgcgtcgcggattgctgcattcatacgtgttgccatttctgctattcctcgttgttgttaattcgaattaactatagccccgctcgcgggggtgtgcaagcggtatttactcaatttaccgattcgcgcccctgggcgcgccaccaggttaggcgtaagggttgttgtctttctcaaagcggaaggcgcccttgtactccggcatggcgtgcatgattaccatcgcgtcgtaccatgctttcattagcggagccaactgtgccccgagcacccgacgatcggcgtaggtaatggccgggttttcgaactggcggtttattgcgtcgatatcttttttgcatatccgcgatcatcccgcgctgttgttgatacgtggtcatttctgcaatccctctttcttgttaagttgatttaactatagctcgataaaatacggattgcaaggtgcaggcgcgcaaaagtgcctagttaccttgcgtgacaattcgcagataacacaacgcaatagccggcgataacacatctgtacttgcaaccaacccgcaatccgctatcacgtcgtgttgccgttttgattaaccaccgtccgctagcctcacctttcttttaaactgcggcctacgtagtgcaaccaaataaactgccgtactgacccagtgcgcattctgtcagtcgtacaggattacattacacaaccaaagatttgcttgttgagtgacccaggcgccttaacggcgcccatagggctaatcaacactgaatcaacaaatacctaaaacaatcggcaacgatccaaaatggtctgccatgagaattacagagtgcatacatacatgctgtgaatacgcgagtgctatggtttattgcaagcatgtatgttgcaaaaccactctcccatttataatgggccccggggcacttattgatttataaggattttttggcgttttaacatatgtacggggcggttatctgggcgttaaatttttgttaaatcactgtatgtatttacagtgttggttgcgcataaataagcgcaaccatacgcgaattatgcataattaatcaaaaacagtgcataagttaagcgcagtaaatttctgatttattgcaaaatgttggttgcaacactttttcctttataaatcaataagtgcccccggggcgggcgcggggcgatttttagcgctgtaaacttttcgcgttggttgcggttttatttagacgtgckaaagcaaccaacattatgcacgttggttgcagtatttgttggttgcagcgtagctaatcttctaaatccgagttggttgcacttggctgcggtggtttttcactatccgataattgttgcggcgcgcaatcccaatccttgaatagccacccggtgatctcaccgtcgtcattcgcggctgggatcactacgccggcctcctgggcctcgcgccatccctcttggaacttcttgcggtcgccatgtacctttttgtataccgagcccgccagggcctgcgatataccctcgggcgcttcgtcaagcgtctgtagcgcctcgatcagcttgtacagtgcggatgatgccttgccgttcttctctttgatctcgttggccatagcacgagcgggcgtgacggtgcccgcgtcgaacggcgccagggcgacagggacaaggtacagtgtctcgtctggcggggtggtcttgaacggcgccggctcgaagccggttagctccttggagaaatccggcgctgctgccgtggattggtattggccgacgagctcggattggttctcgtctaccgggatcttgcacgattgcaggatgaacccgcgcggcgattggcgcatgccaaaacgcgctttctcgtggtagaaattgagttggtgctctttatccggatcaggctgctcaaggaagaaagccgcatcaacggcagcatgcagggccccactacctcgcgccgtccggttgccgttgttgttggacttggccgggtggtggatgacgcccggtgatccgcctgtctctctggcgatgtctttcaggcaggccaccactttacccatatcggtagcgttgttctcgtcgaatggctcggcggcgagcgcggtagtggcgttcagcgagtcgaacgtgataatccctactggttcttcccccgccaggtcgttgatcaggcgcacgcacttcttgcgccccgctggggtggtaatgtcgatcccggcgccagcggtgtcgatgatgtgcaggcgggataagtcggactggtattttatctgtagcgcttttttacggcgcttggattcctccggcgcttcggcgtcgaaatagaaacagtgcgccgggatcaccgccttgccggcgaattggataccagccgcgaccgccgccatagttccgaggatgtggaaagatttcccgatgttcgatccgccggccgcgtaccaggtagagcggaagtttagcaacccctcaatgatcgggtcgtgctgcgtgaactgcgggcggctcggctcgtcttccagatcctcatcggtgcagacgtacaagtcctcgtctgctgccgcgtcgttctcggcctggtgcagtcgctgcaactcttcatcatcgattaacggcagcgcttcggcgagttgtgcgcggctgatcggatggggtgacggtagcaggtagtcgggtacgccgagcatacgcatagctaggtgctggtggcggttgatgttaccgtgcccggagcagttggcgtgctggcagtggaaatgcacctcggggtgcttggcattgggtaataggattgccgtggagctggtcccgtccgtgtcggtggagtggttggccgagttcgggcactctacaatgtaccctcggcggctcggcatgagttcgagccccatttcctcgcaccactccatgatcgcacgtccgttctcgtccgctttggcgaggtcgtcttctgatagcgtagggcgatccgatttttcggcgggtgcttcccacgccatggctaaaacgtcatccaccgccagggtgcggccagcgcccgtgcgaaatgccgcgttgcgatgcggtacgaacatcaagcgggcgcgctgataggcggtggcgtcggcgcagtcccacacgtccagcatatgcgccagcgtggtgtttacctgccagatctcatccgcggccatcgggcgatcggtggggatagcaaaacgcacgctgcgggtgtcttcacctttcagcgggtggcgatcacccggggtggtgtattcgagatagcgcaggttgaggcgcgacaatgcgcggcgcaccaggaaaaggcgtgctggtgtgacggcgtccaggtcgagccagcagatagatcggcagtctacgccatcgtcgccgcgcttggcgccaggtagcacagatgcacagatatatttttggcggctcttcttggcccggtattcgtcctcggtctcgctgccggtgaacgtcgggcggttctcgatttcgtcggggcgcgatgcgtaaagaagtcgaagagctggcccaggtattcgccagcgcggatgatgatgaagcgctggaggagttcaaaaaactcctggaagatttcggcgagcgcaccgtgaagaaaatctctgatgacgacctgccgggcttccacgaggagctgaaaaaactggccgatgagtttttcgagttcgaagaagaataacactacgctggcccggttatcgccgggccactttttagaggtgtaaaaatgagtgcttataattgggccctttgcgatcttttatgccgtagagatttcgtatggccgcgcagccttgcacaaacagtgtcgtcacgcactcgaccgccaaacggttggcgttggagtcttgtcgcgacgggtgatttcattatgcgtgggccaactgacgatttggttatgtacaaagacaactacgatgcggcgttgatgagtttgagaaacaaaaaagatatcaaaaaatacgaaacgtacacctcggctaaaccgtcaaaaagcgccgccgatatcctcacggcggcggcggaccatatggccgagcgcgccacacaacgcgatacacctggaggcgagcgcactatgtctcgcacggtggccgcattcaacgcgatgtacggcaccaacttaaccgaggtgcagggctggcagtttatggtcctgttgaaaatgtcccgcgcttccgcgggtgcgcatgttgccgatgattacgaagatcagacggcgtattcggcgctagcgggtgaatgcgcaaatcgggaggattagcgatgcattatcaactctatatcggtactgatctgcgtgatggcgcgcaagcgttatggctcctgcgcggtccggtcgatgcgataaccgagtgcgtggcgttgtcaccgaaagtatccaacgtcgatgtgatcatgaacacgcgccgcgagcgtgatccgtatgagttcatggcaatcgccattttcgaaaagcatgcgcacgccgtggcgccgcttacgtcctgggaggtttaaccgtggccgtcctgaaagcgaaacgcaaaaataaagatcgctccggtagtaacgaggagcacgcgctattgtcgccaagctccgctaaaaagtggctcggctgtcccgcggcgctcaccgctgaaatcgggatccccaacccgtcaaatcctgcggcggaagcgggaaccgcgatgcacgccgttgccgagattatggcgaataatttgatccgcgatggtgaaagcaaggctgcgtctgaattcgtcgggggctacccgctgcataccccgacgaagaaaagcaaggggccgaagttcaccgacgaaatggccaagatggtgcagggctacattgacacctgcgtagcgcccctagtcgatgccggcgccgaagtgtatatcgagtcgcgcgtagaccttagccgcccgctcggcgcacctaacactttcggcaccgcggacttagtggccgtcacagagctgaccgacggatcgaacatgctgatcgtcggcgacttgaaaaccgggcggcacccggtggacgccaaagaaaaccggcagatgatgatctacgcgctcggtttgctgaataaatatcgcttctcgcacgatatcaccaaagtgcgcttgatgatttatcagccgttttgcggtggcgttagtgagtgggacacgtcggcggaagtcatcgagacgtttggcaagttcgcgaaagaccgcgccgctaaggccttggcgtgccacgccgccggtaaagccgcgttaaagcctggcgacttccggccatccgccgatgcgtgtcagtggtgccgttttcgcgagaagtgcaacgcagcgcgcaagttcaacgagcagatcgccgctgacgacctacgtgatgagtccggcgacgaaatgacgccagaggagctggccgaggcctacgccaagttaccggcgctgcgccagcacatcaaaaacatcgaatcggcaacgtataaggcgctgttagccggtaccaaactgcctgggctgaaactggtagccggtaaggatggtaatcgcacctggtcagatgaggcgcttgtgcaattgcgtcttgagcaaggcggcgttacgccggatgcgatgtacacgcagaaactgctaacgcctacccaggccgaaaaagcactaccggcgggcgcgtttgagtgggtggaagaactcatcacccgcaagccgggcgagccgtcgatcgcatcggcagacgacaagcgcccggaatacgtgccagttaaagacgacgatttagtcgattaaaaattggttgcaatgtcctacgtgttgtgacctaatacataagccgacgcggcggcccttaccgcgataaaaatgtgaattggagagtgttaaaatggctaaagtcaatctgaaaaatgtccgtctgtgtttcctccacgctttcgagcgcgccgagccgaaaaacaaaggggaaaaggccgcctacaaggtgtgtatcctcctggacaaagacgatcagcaggttgaaaaactggaagacaccgcgttagaggtgttaaccgcaaagtggggcaagcgcgaagttgccgagcgttggatgtcgcgtaactatgcgcaggatagcagcaaggaatgcgccgttaatgatggtgacctgcgcgaagaggttaccccggagtttgaaaacgcgatctatatcaatgcccgcagcccgaagcagccgaagattcaaacgtctttaggcgaggaccagaccgagccgggtatcacggttgatggcgatccgatcgagggcaaagaaatttacgctgggtgttacgctaacgtcagcattgagttgtgggcccaggataatgaacatggtaagggtctgcgcgctgcaatcctcggcttgcgtttccgtgccgatggtgaagcgttcggcggtggcggctcaacggcaaccgatgacgacctgagcgacgatgatgacgagccgcgtagcgtatcccgccgccgcagtcgtgacgacgaagatgacgcaccgcgcggtaagtctcgcaaccgtcgtgatcgcgatgaggatgaagacgatgaaccacgtgagcgccgccgtagcgtatcccgccgccgcagtcgtgacgacgattaataaaaagcctcgatagtacctacggcctcgcatgaggccgtttttctaagggccgcattatgccacaactcctatttcttgacttcgaaacattcagtgaagccgatttgaaaaaagtcggtgcctatgcctacgcagagcacgattcaaccgagatcctgttagcgtcatacgcgtttgatgacggccccgccaaagtgtgggacgctacttgcgcatcaggcgaaagcgatatcgatctagataacaattccgcccccgatgatctgctgcgtggcctgcgtcgtgcaaaacgcgggcgcgtcaaactggtgatgcataacggcttgatgttcgaccgcttgatcatccgcgaatgccttggtctcgatatcccgccggagcacatccacgatacaatggtgcaggcgttccgccacgcgctacccggcagcctggataaactgtgcgaagtgcttaacgtcgatgccgacctggcgaaagacaaagcgggtaaggcgctgatcaagcgattctgcaagcctacaccgaaaaactacaagatccgacgctatgaccgcaacacgcatccggacgaatggaagcaattcaagcactacgcgcgcaacgacatcacggcaatgcgtgagatctactacaaaatgccgtcatggggcgagatagacaaagaaaacgagatcttggcacttgaccagcgcattaacgatcgcgggttttatgtggacactgatttagctaaagccgcgaccgccgcggtggccgctgcgcgcgctgaactgcaggaggccgcgcaagcgacttacggcggcggccttaccggtgccgattttctccccctcctgcgcgatctggcacccgcgcatcacatcccaaacgcgcagaaatcaacgctcggtgacctgctggatgacgccgacttacccgacgaggcccgccaggtgatcgaaatgcggctaggcgcggccagtaccgccagcacgaaatatgcccccctgcttaatggtatgtccgccgacggccgccgccgcgggtgcctgcaatatggcggcgccaaacgcacactccgttgggcgggcaagggctttcagccgcagaacctggcacgcgggtatttcaaagaaaaaccgctagcccgtgggatcgaggcgctgaaacgcggcaccgcggagtacgctttcgacgtaatgaagctggcggcatccacggttcgcggctgcatcatcccggcaccgggtaaaaaattggtcgttgccgactactctaacgtcgagggtcgcggtctggcctggctggcgggggaggattcggcgctcgatactttccgcgcggggttggatatctacaaagtgaccgccggcaagatgttcggcatcagtccggacgacgtggatggctaccgccggcagatcggcaaggcctgcgaattgggtctcggctacggtggcggcgtggccgcgttcctgacattctctaaaaacctcggtctggatctggaggaaatggccgttacgatggctggcactttccctgattaccactggcgcgccgcgctacgcgcctatgaattcatgaagttgcaggaggtgaagcgcaagccgctacccggtaaaaaagacgatcgaacgaccgtcgtcctctctaaaaaagcgtggcttacatgcgattgcatcaaacgtatgtggcgggagtcgcacccaagaacggtgcaattctggtatgacctggaagaagcctgtttgatggctatcgacaatccaggggcgtcgtattgggcgggggccaaggttcgccaagacggcaaacgcgccatacgcatcgagcggacattaacgcggtctggcaagccgggcaactggctaaagatcgaattgccgtccggacgtatcctgtcctatccggggatcggcgtgtcgatggagaaaaccaacgaggacgatccgggcgagaaagcgcgcccacgcatcaaataccgtggagagaaccagttaacgcgtcaatgggggtggcagcacacctacggcgggaaattggcggagaacgtcacccaggcgctgtgccgcgacatcctagcatggtgcatgctgcccgtcgataacgcaggctatgagatcatcctgtcggtacacgatgagctgatcaccgagacgcccgatacggcagaatacaacgttgccgaacttgagcgcctgatgtgcgacttgccagcctgggccaagggattcccgctaaaggccgagggctgggaaggataccgctacaagaaatgatgggggttgtatgacgcccgaaggtaaagtgcaggcgcacctgcaacgacggtttaaggcgatcggcggcttggtgcgcaagatatcctatgaggggcggcgcggctgccctgacctgtttatcgtgttgccgggtggggtggtggtcatggtggaggttaaaaagcctggcggtacgccggagccacaccaggtgcgcgagatagagcgcttacggcaacgtggtgtgccagtgtatgtaatcgacagtatcgagggtgcggataagttggttgcattttatagctgatttatctatagttggttgcaaggacgcaaccaggagcacgcacaatgcatgacatcttcg

SEQ ID NO: 3 - DNA, 42923 bp, Bacteriophage phi eiAU
catcggtacacgaagccgatcaggttctgcggatggtatctgacgatattacaaccttacgcgatgtgcgccgcgacgagtggcgcaatgcccgtcgcgcgggcacaagccgcgatcgcgaattcattaaatgggatgagaatgtgtcgctagttttccaaaaactttgcgatttgcgcgatagcgcacagtttttcatcgacacccatacgccaccggctattgccgagctttatttccggagaatttaccgtgattagaaaagtgcgccttaaaaaacgcatcctgaaaatgtgccgctgctgcggcgtggaaaagccgttgtacgaattccacaaatacaccggcaccacctgccggtcgccagacggacaccgggcgatctgcaaggtgtgtcgcaatgaacaggcccgcgagtatgcgcgccgtaaacgtgcaaagaatggagaataaaaccatggccactattaccaaaaaacaacgcgcagaacttcgcatgaaatttggtggccgctgtgcttattgcgggtgcgaactttcagatagggggtggcacgccgatcatgtagaaccggcattgcgtaagtgggagttcgttaaaaataaaacaagtggagtgctacaaactgcttctacgggggaattttggcgacctgaaaatgatacgctcgaaaacctgttcccatcctgtgctccatgcaatctatttaaggcaacttttagtgtagagatgtttcgagaacagatcgcagaacaggtaaaacgcgcacggtcacgcagcgtaaatttccgcacggcggagcgattcgggcttattaaggttattgatatgccggttgttttctggtttgagcggtatcaggaaggagcagatcaccaaggcgatagtagaaaagctagccgtaattgggaaaggtactcatgatgaatcacaaattattgcgccatcttggctacggtgaattcccggacgcagtaatcgatgccgaattgtgccgagtaatggccgcgaagtacaaaaactcaatccccggtgccctgcgccatttcgcccgagcgcgagccgcaacagtgcgcaatccgtcgctaaaatcggcactggtcgagatgggcggcagtatctacccggaaaccgggatcgccaccctgcgcgcttgcctggacaagatgcacgccgctgcggtgcgtgaactgcgcgcgcaaggcattacgcccgatgaatatatccgggccgcggaggagcaacatggcacagtttaagcgccgcccgtaccagaaagcgatcacgggccacatcatcgcgcatgctcggtgcaacgtgtgggctacgatgggcagcgggaaaacgggcgcgacgatgtgggcgctagatgccatgtttagcaccggcattctagatgagtcggatcgcgttctgatcctcgccccgttgcgcgttgcgtctggcacttggccggaggaacagcgcaagtggaaatttcccgcgctgcgggttatcgatgccaccggtaacgccgagcaccggatcgaggcactggcaacatcggcgaatgtggtatgcctgaattacgacgtgctggaatggctggtcgagtattacggcaacgattggccgtttactgtcgtagttgccgatgaaagcacgcggttaaaatcgtatcgtagccgcggcggtagcaagcgggcccgcgcattggcgaaagtggcgcataagaaaatccgcaggtttatcaatctgaccggtacgccagcgccgaacggcttaaaggacgtgtgggggcagatgtggtttctcgatgcgggcgagcgccttggcaccagttatcaatcattctcagatcgctggttcgtcagtaagcaagtcggctcgtcaccacttgcgcgccagatatcgccacgcaccggggcggaaaccgagatccaccagaagtgcgcggacctcagcatcacgatcgacgcggcggagtatttcgggtgtgataagccggtagtcgtaccgatcgtagtcgagttgccgaagaaagcgcgcaagatctacgacgatatggaaaacgcgcttttcgctgaattggaaagcggcgaaatcgaggcctcgaacgcggcggcaaaaacggccaagtgtttacagatcgcgggcggcgcctgttacatcacgaccgacgatggcgaggcatccaaagagtggacggaaatccacaaggccaagctcgacgcgctggaatccatcatcgaggagctaaacggcagcccgttgctagtcgcgtaccagtataaacacgacctggtgcgcctgctaaaacgcttcccgcagggccgcgcgatgcgcaaggggttaaagggcaacaatgacatggccgattggaacgccggcaaggtgccgatcatgttcgtgcatccagccagcgcgggccatggcctgaacttgcaggacggcgggtgccatctggcctttttcaacgatacgtggaactatgagcaatatgcgcagatcgtcgagcgtatcggccccgtccgccagcaccaagccgggcacccgcgcacggtatacatatacatcattcaggcacgcggaacacttgatgaggttgtcgccctgcggcgcgacgacaaggccgaagtgcaagacctgttaatggactatatgaaacgcaaaaagagaggcaaatgatgtcccgcatgctccgatctaatcccgtcgttgccgttcctgtaggtggcggccccgccatctacttaccgtgccccaaggctattatgcgacgcggttttttacccgctggcgttacccaagtattgcttggccgcaaaaagtcgcatcgcgggttcgtattccgcagggcaaccaatcgtgagatcgcggcgttcgattgcgatatcggatatctagccccatcagagtttagctacgagctgttggcctcactgctgacgtaccacccgcgcaccggtgagatccgcgataaacgtaccggaaagcgcaagggggcctctaccccctccggtggggtaacagtcatcgtgaacgataaaacgatgtgggggccgcgtgtggcatgggtattacacactcggcaacctgtgccggatggcctgacggtgcgctgcatcgacggagggatcggacattatgcacagcgttggaccaatctggaattatgcaaacaggaagatattcgccttgacgaaagcgcgatagacggctacagttaattcgatttaacaaccggtgccgaatatatgactgcctattacaatgaaattgacccctacgccgcgcaatggctgcgtaatcttatcgcagaagggcatatcgcccctggtattgttgacgaacgatcgatcgaggatataacaccaaatgaactcaccgaatttacccagtgccacttcttcgccggaattggagtatggtcgctcgccctgcgccgcgcaggatggccggatgatcgaccggtctggaccggatcttgcccttgccagcctttcagcgcggcaggcaaaggcgcaggggttgctgacgagcggcacctgtggccggcattcttccatctcatcagccagtgcagccctagcgtcgtctttggtgagcaggtttcaagcaaggacggcctcggttggctcgacattgtacaaactgacttggaaaacgcgggatacgccagcgcagcggcagatttatgcgctgcgggcgtcggtgcgccgcacatccgacagcgattgtactgggtggccgacgccaaccaccagcgacaggaagggaagcagccccgccaccatgcggaaggatgggaaggacaggactttctgccgtctcgactatgcgacggagcaggggttagtagtgtctccgccgagagcggacgggtcgcaacgattcgatcaattaccgagacagggcaatctctgcgggtggccgacgccgagggcggtagatggggagaaaaactccagaacattggcgggggcactacgggagttggagcgcgggaaattatcttgcctgccggggtggtcacaaatgacgctgccgggccgactaacggccactggcgagattctgactggctcagctgccgggatggaaagtggcggccagttgaacccggcacattcccgttggctaatgggattacctcccgagtgggacgattgcgcgcctacgggaacgccatctgtgcgcagctcgcagaagaattcatccgggcatatctcgacacggagaaaaattaaaatgtctggctatcacgattcaaaaacggcaccagaagataaagattgctggcgtaccccgccggaggttttcaggtatgctgttcgtacatggggttctttcgaaatagacgccgcagcggcagatcacaatcaccttgttgccgattactggacgctagcagataacgcgctggtgcaggattggagcggaaaacgtgtatggtgtaatccaccgtatagcgacatcggcccctgggtagagaaagccgctacggcggaattctgcgtaatgctagttcccgctgacacgtcggttaagtggttcgccaccgcgggagaactcggggcgtccgttatttttatcacgcgtggccgtttgcggtttatccataacgcaacgggaaagccggggccgagtaacaaaatggggtcttgctttctggtctttggcggtagtcgaccaggacgggtagatttcgtaacgcgggctggcgtttatcaaatcggcgcacgccgcaaagtgacggttaaaaggcgcgtccgtgcgccgcacaatgcaacataattttaacacaataggccgctgcgtctaccattaaaaaaaaaatggttgcaaagttggcggcctacgcctatagttaaatcaacttaacaagaaagaggaatcgcagaaatgaacgacgaactcaataaattgatagatctgattaaggcccgagacgatatcgcatgcaaacttagcgcactacatagcgaaatctgtagccttacggtagaacttaaagccaagaatagggcaattgttgaaaatgcctgcgctttgggtgggtattcagtggaagaatatattaacagtgacggtatgagggccttttaacgatgaaacgaatcaccgcaatcgcaatcataaccgccgctatcatcggcagttcgtatgtcggcactgtaggcgccgaggacatgaccgcgcatgataagtgcgaatacctggcgtataacggcccgtcggcaccagctagtgcagacgaccgcgacacggcaacgcttctatgccttaacgccgtaacagttgccgaagaaaaccccggcgtatcggttgacgtcctccgcggcattctcagcttgcaaggtgcgatgcagcacaacccggaaaaagaagccgatcaccgctggcgttcgctagccatcctgcacggtttcaacatccaacgcggcaattacaatacgggcggtgcaaaatgacctacttccttgccatgatcgcaatcatgctgacggcagtcactatcggcactatcgacaagaaagagaaagggctgtcggggctatctcgcgggttgctgcgtgtgctagcgatggccgcaatgtgccttagtttcttcgtggcgttcgatatggtggattttaagagcccgtattacgggtacgtaaaggatcagcacaagttaacgacagcgcttgtttttggcctaggtgccatcacgctatcgatcatatcgacgttcggaaagcgtaaataaccaaggggccgctaggcccctttttcggcttcatgcattcccattatcgcaagtgccttaaccgcaatccgctgtaggcgctccatctgtgccgtgttgagcttcccggtagaggccaccgccataatacccgataaactccctagcgccttaaaatcggccatggcggattgtatgatagccgtctgcgggtagccctcgtcgagtatggcctgcgcccgttccgccagattatcggcgacagctttaaccccgtcactcggcgtcatcttcgcttacctcgcctttttcttttcccttttgcggtaccgctgccggttgcggagggaaagggctgatcggctcgcgctgttcttcctgcgcggctggcggtgtgatatctaaaaattgtttgatgaatgacccagcgaaatcgatatagtcgcaagtgtagacgtgcatgcggtcttgatcagcgggcgtcaacatgttggggctcatcatgatcttgtgttggtggaaaccgacatagaggacgttaatcggtgctttgatcgatccgggcggcggaacgataacgcccaaaatccaacaataatgcacctggccatttccgaggctgatcggttcgcaaccgacgttcaaccactgcacctccgagtgcgggaaagttgcagcgatatctgttgcgtccttccgcaaatgccaatctacgccggctatgcggatcacttttttaggcgtcatcccgttgaactgagtcacgcccacgccttggctttggtattggtttgtcattgtgtcggctccaccgtgtttttacatcttgtgcattttttctagtgcggcaatacacgccgcggctagatcaacgagttcgtgctcgataccttcgcgcgacccttgctctttcttggtcatcagctcatggtattccatttctacaacggccatcataccgcccggctgggaaatatgacttgcccaagttgcaggatgttcatgaactcgttcccgtacactctccataaccgcactatgcgaatcatggtgtttcggatgacgataaccagtgacatcttccataacctccgtgccgccggatagcggatgatagtaatgatgatgttctgacaccggcgcgccatggtggcggcgcatccggcgatacattaatactcgcatttttacacctcgctaatcgcccggattgctccgggcgcgggttagacaggcttacgcggatttggtgccgagatgtgccaaaatcgtgttgatgatctggttagtctgggccgcctggctgttttgcaggaagatttccccggacagcttggcgttctgcgctttctggtcgcacaactgagtttgcagatcggtgatgaagttctgctggatcagttggcgagtggcggcgccctccgccgcgatggtcttctgcgtctcgcagcaacagcgggccatatcggcgctgacaccggcgaaaccgctggccacggcaaagcgggtatccgcaccgttgcggtcaacggtggcattaacccctgcgaagccctggcacaatgcggactggatgcccgcctgcccctggaagttcgcatactgagccgcagacagcccattagcgatcgtcatgttggtgccgttctggccttgcagagtttgcagggtgcccgcattcactgcggacgcgacggagttgataccgtccagcacagcggtagtcgcgatagccgcttcaccggcaacgccgccacggttaccccagccgccgccaaagccattgccgaaccaagagccgatcaggccgccaaccgcaccgccaagacctgcggcaccggcttcaccacccatcccgccggcggggatcagagtcatatcagacatgttaacacctctttgtgtttgtttagtttttgtaacaactaagttacacaaagagaataccccgcagatcagcgcttgccaaccacacagggtattttttagtgcaaggggttgattaacttggagttataatatcggtgtttatcggtggcggggtgaactctcccgactcggtgattttaaatcccggccctaccggataatcgcgtaaatcaatttcgttttcgtgtaagtgataatcgaggtctaccacatgcacgttaacaataatgccgttttcgattatgcctatccgcatacgtacctcaacaatattcgtagattatacacacgccattacttccggccccactagctcccgaactcgttccgcccgttccgccgccgcctccgtatcccgatccgggtttggcggtcgagccgtaaccgccagccccaccaaaccctaataatgacgacccgcctgatccacctctttgattcaccgtattttgactggcttgctgcccgcattgcccgggagtaataactccgcgtattgcggaattataaataattccacctcgaggagtcacggaatctccctcacgatatccgctgccgccgccgtatgcggtgattagattcccaaaatagctatcgccacccgtcaagccgtccgtgtatcccgtaaccgaggcgccccccgcgccgatggttacggtgacaggccctgttaatagtgaggccggtatttttgcttctacggtccccgccccgccaccaccgcctgcgccgagtgttacggaagtttgtgaagaaccactaccgccaccaccgcacattttaacgatacagtactttaaattctccggtgcagagtatgccgacgtagtggttagcaccttaatatcaaacaatgatactgcggttaatccggcgatagtattatctacgtaggtcttgtctgcctttttgtctaactcggtatctacgtaggtcttgtctgcctttttgtctaactcggtagtgattttctggaagctgctgcccgtatattgtacgccgtctatttcgaccgtgatatccccggtggcagaatagaactgttttagcgcgtcggattgctcctgataacttcgcaatgtcgcggcgagctggcgggcaaagtccgggcgtgactgcccgtagaagctaagtattgcatacgcagaccctgacggtaagttgctggcgtccgacaccaatgtcatcgacgtgtcgccgtttacacgcgctatctcatagatttttaccacaccggacgacggaacgagaagcgcctgaccctctcctatacccgcggatggatcagcccattttgtacccgaaccggttacctctttgccgtttatggcgattgttcctgttttgtaccatgccattgttatgtattcctatggttagccctctaaaatttcggcactaccgcgtctagcccgccccattaataattgggcaccgtcatatgtgagcagtgtatgcggtttttcctgagtgattgtatgaggccaagtcttggtgaactctattcgtatccatgtgccgttggcaccaggcggaagagtcaaacccgatagattaaatttatattccgccgttaccccgttagaccataattccgtcaagacttttcgcactccggaggattcaacgtaacatgatattctagtcataccgcccttaccggcaaaaaactttacatagccattggatataatggtctgttcaaaatcttcagcggggaccttaaataggactcttgtggcaccattgcccacgcttacatctttacttgaaaatgtaaagtttgagtattgtactacgtcgccaactattctattggcgtgcaccgtccctttaaaatcgccattttccgcgtacacagtgccgcgtatcgtggcgttattgaactctacggccccagatttattaatagtccagccggcgccacccggggtgtagtcatccgattgaaggacgcccgcaattttagcactaccgatcgaggcgtcacgaatgaatgcgtcacgcagatatgcctggtcaccaaccaccccaaacataagttctggtttatctcctactttagccataacggcaaaacgatcggccagcatggcgacttgcgtagagacctcgccctctttcacctcagcggaaattgacatgccagcagaataatattccccgttgtatgttatccccgcgttagtatcccataccgcgtaaccgttgccgtcgtaatcgaacttcgctgtcatttttgttgctacggctgcttcgttttctgccaccgaggctttaaccgcagttaacgattcggccagggcgccaacttcattcaccacgacattatcgattctaacgatctctgcggatagttcgcctaattttttggattttcgttgtatgtcgccgtcattggcgattgcgttctgctcaatggcatctatcgcggcattaatgctcgtctcgagcgccttacctccttcagatgacatgacctgatccccgagcgcatccataatcgaatccgtgtcgattgatgcccgcccctgtacactggcgccccattccgatttattgccgaggcggtcaactaaacgtgcgcgataccaaaagacggcattagctttaagaccactatgggcgaaggacttgctcgggtatggggataaactaagcgccgtaaaggcttcttcagtgtttgtcggtgcgtattggatctcagtgtattcggtgtcctcggcatctttggggaacgcccacgcgacatcaataccaaagactacatcgctagatgctctcagggatactggggcgggtacttcaccctggcggccgtcgatatgcgttaatacactggatgcccacacactagacgccccggaagacgtgaccgcacgaacgcgcaccagataatcgccggcaaatacaccttgtacctcaaaaccatttacactgctcgccggcacattaacccaatcattggcccctctgcgccattgtgcttcatacgcgacaatatccggctgtactttaccgtcggccatgcgcgacggctcccaggtagcgcgcatcgtcgttacacgctggttttgtcgcacctgctcgtaactggtgatggcgaccgcggtcggtgcgttgacaattccggtcggcaaaaggctaactggtggcttgtctaacctggcgccatcgtctacggcatcatatttcgagtcgttatactccgctgcgctaatctcgaacgtgttattttcgtcgtcgaaatctagcgttaacacgcggaatttttgcaacgccaattcgcctgagtcaactacccaaaccgcatttaccgcgggcgctgccgtgaatggctcggcgatagttacaacgctatccccgaccgaagcaatttttctggactctaccgcgccgccagtggtgcggatcagcaaatcatcgcccggtttaccgtccgtagtacgatcgagcattacctgtttggttttctcgtcatatcccgcgacgcggccacccataacacggcccccgatccgctcgtcggccaaggcgaagaccgtaccgggcagaaaggcgaaaccctccaaccctacgcggagttttaccagccgatctaccgagttggtaagcaccgcccatgatgcgcggcgctgcgcttcgctttcgcgcgtacagccgatcgctgtcagttgggtctgttcgaaaccgagttgcgccacaagctcctggaacataacggccgtcggggtatctgcgtaatggttttgggcgtcactataattaacaagagccgagctaaaacgcgttttacggctgccgcttgaataggttggcttgccgatgatagaggcgcgggtgacgatctgcgatggtgctttcaccggcatatcggaaaccacattgaacatgttgtttccccaatacgtcagcccgttaaatcccgccgcgatatcacggatcaccgtccaggcatcggcctgcgcctgcaaataacagttgaactcgaaacgcggctcaacaccaccggcaccatccggcaccatctgatcgcagcgctgtgcgatgcggtacaactcccatttatctagcatggccgccgtaacgcgcttaccaagcccgaaccgcggttgagtcaatacgtcgtaccatacccacgccggattgttggtgtaggcccatttaaacgacccatcccaattgccggaataggtgcgggtgtctgggtcatagttacttggtactcggataatacgccctttggccaggcacgatattttcgggatattctggaatgattttgcgttgaactcgataaacaacaaggcggtatgcgggtatcgaaaacgtgcgtcgataacctccgtgatggcctgaattttaagcgtgttggcgagacgtgcgctatctgaatccggggtttcacgcacaacgcgaatccgccagccggacgagctacgcggcaggttgacacgtatcgaccgctcatagagcgtagttgttttacctgagatggcgaatttaccctgtgtactaaacgtgcctcccccaacggcaagatcaatccggaacgataccgaagtgccgactacgtcgccgtcatcctcctggtacattagcgcgggaacgcctacgcgaacgacgacagcgtcgatctcggttctggttagcgcatgcacccacgggctggcctgtttaatttcagtaccgaagcccgtctcgttttctactgcggggaaacccgagatcgggtcctgctgctgtacgccagggcggacttcccaacgcacgccgtcgaagttacgcgacccgtccggattgcctagcggcgttccgtccaggaaaattttagtgtcatccaaccccagcgccatttccccctcgcctaatgcgatgagtagacgggcctttgcctggctacgaatactatccggttgctctaccggtgtatgcgcatcgccgccaccaccctttgcgcctttgatgtaaaaaagttgtctctgcattatgctacgtcctcggcaacaattccggcactgattatagcaccgccgatctctctcgtcccccatagcactccgatcgggttacccattgccgtggtgttaacagggccgccgaaagcgtaggatggcctattatccggatcctctcgcgattgtaatccgcgcggctgcggggatagcatctggtaaaggcccccagccataacggccgcacccgcccaagccatttgcgcaccgaatgacgccaacgcaccacctgagaaaatcgtaagacccacgcccgccacgacaagaacggcgccgagaatggtctgaaatagaccgccttttttcgatccctcgatcaccggggcgatgcggatctcatcgttgcctatgccatcgtgcaattcttccgcgccgatgtttttacgtccgcgaaatacggcgaacgtcatgccgttctttttggcgtgcaagagatactcctcgaatccatcgagcgttacgcacagcgcctttaccgcttctgtcgtggtcgacaccgcgcgacgatgcacccggccaaatgccgcgcccagtttgccgtacaggcggattgtgattaatctctccacggttttaattcctccggtaaatctcgatgacggatgcataacacggtgcggtccacataatacccgccatagggtatctcctccgacgcgcggccatataggtggtgcaaaagcccacactcggtaagtacaccggcatgattcggaacatcggcccgcacctgcatgatgacgacgcagccgggcgcaggatccgcctcgacgaaacccgctgcggcccaattatccatgtataggttttcgccgcgctcccaccaagggtaatttacacggaaatctaataggtcgatgccttgccttttatgccaggccatgactagcccgtaacaatcatcggcgcccaacacgaacggccggccgattagcggcggatcctgcggtgagatctcgcggtactcgtcgcaatctggcgcatataccccccatatgattccgcttgcattacacgcggccttatcagcatctgatggctctgccgtcgccccgtcgcccgggtgactgtgtatgacgcgcacaatgtcgccctcgtcggcagccgccgcaaagtcagccgcagagatccggaaatgttctgtcggcatttcatgcgtattggtaactctgatatatcgcagcgcgcggccattttgtaccactaggccgcagcattcgttatagccagactcggcggcgtgttttttgatctcgcctagaataattttgttcatggcatcaccggttgatcagttgggctgcaaggaatccgccaaaatccagtgctgcggccttggggtcggccatatcggccccgaagcgcttaacgcaatcggagtaacacccaccacagcgatcaagcgccgggtctgaaacagggttgcccttagcgtcgaaatacgcgttgccgttgtaggtacagccgtcgccgctgcggtactgtccgcgcatcgcccaggtgcatagcgatgtgatctgacgtgtcggaatttttagcccttgtaaatccgccggactgcttaacgtccaggtgatcgactcgtcatcctcggctgatttagtatcgacccaaaaagtttgataactgcaagcgtccggatcggcattggggttaccgtcggggaagttgcgcgcgtcaaggtattcggcgtaggtgtaaatcacacgtactttcgcgtttatcaagtcgcgataatccagacataagcggctcaaatacccggccaagttcgaaaccgtgatcgtaggtgtcgcggcctgttctgtagacagcgacaacccggacacgctaaacggccagaagtcgaacattaacccgccaaaatatatcggcttcggccctaacttagcctcgtcgccggccgccgcttcgatctcggcagcggagtgcgggaagggggcataatgcatgcggtgtacgccggccccgaactcggaggcatccacctcgactaaaacgatacgcccggacgggtctagttttgccgattggtctatgtacgacgtcatgcgaacaccccatacgcccggttcagcgtgaaggtaacctcggcggtatgccccgttaggttaaacgccaccgattcagcatcgacgcgatacagccccttttcttcgccgggcggagtgaagataaatgccttgacgacgtgccgtaagaggaatgcccgcacctcccgcattcgagcgttgtcaccagcgcaacgcaaggctatggtatcggcgacggagttgataccgttttcggctacctgttcgtagccgtcgcccatcttggccgagcgtaccgattttttataggtgatcggcgcggtcaattcgcataattcagtaaaagtttctacggtcatctaaaaatgcctccgctatttctggaggcattttaacaaacgttggttgcactgtctaatcacgccatccgcgatttgtatagcaacccgcccggctgtagcatacgcgtaacttctgccgttacggtatccttcatctgcttggcgatcgacgcgccaagggcaccgccggttgacgttgctcggcccattgcaccaccatcagaaaccgtaaccgagatgttattaatgatcgatgaacccgttccaccttctgccgatatgcctaaacgtccatcggcggtgcgcttcaacggcataatggcctcaggtccggcttctcccatcaacccggcgcccttggcgaacttcgggacggcgtcgaaattaaacatggttggctggtttaccacctgcccagaataccgagaaagatcaccaccggaataaacgccgcctttcgcgttagcgaacaacccgcctagccccaatgcattagcagccgatttgattccttgcatcactaatagttgcgttatgatatcggcgatcatctttagcacgtcggcggtgaacgacttaaaatccattttgccggaagtcacaaaatccgagacggctttgacaccgcgattcatcgcatcctcgaccgcttggcccgcaatagctgaataattagtgctggcatcggcccaatctttaagaccgcgaatcgcgccagcctgccaatcttgttcggcgatatcctgctcggcccataatttgcgtttcgcgtctagcgctttctcgatatccgccgttgtggcgccctgagcggccaacgtagcccgcgttaccgcttcgtcgttgaaccgcttcgcctctttggtgctcatgccgtaggtggctgccagggcctgcatttgtgcatccgtctcggcgacggcttttgacacgttatcatgcgcctctgcctggcgttgcaacacaagcagttgatcgccggcatctgccagttgcaccttagacgcccggatctgctcatagctcgccatgatttgtttttcggcaagtgtcagttgccgtttttgcgaggcttccaccaacacgctatgctcggcctcaaatagcaggagagcgcggcgctgctggctggcgttgcggtcatacgtgtcacgctgcttcatcaatgcgatctgcgcttccagcgcgcgtagttgggcggctgactgctcgtcaacacggacgccggcatcgactttaacggccttggcgcgtacaggcttttcctcatactttttgcggatctcctcaacccgacgattgtactcggccaggctgatcaggttggcatctagcaggcgcttctgtgtgtcgatctcacggttttgtttctgcgtgttagtggcgaacttctctgcctccgatgccgccgtcgcgttgagcacgttttgtttttgctgctcctgggcggccgcctttgtcgccgcagcttttttattctcggcctgtgtctgctgccgtagcgtttccacctggcgccgggcagtctccatctggtcatatagcgcggcctttccggcctccgttactaccttgctatctttttcaagctcccagcgctttttattcgccgtgtataccttctcggcagcggccaattgcacacctaccgattccgggcgcccgatatctaagatagcgtcccacatattcttggcggcgttacggatcgacatcatagcgagctctaccatgcctaactgcccggtaacattagcggcaagatcagtaaatgacgctgaggcgatgcggttggcctccgcgatggcccgcgtttgctgcccctcgtcgatcagcttctgaacacggataatctgctcttcggtcaccgccttgtattgcgtctgcaaggcgcgtaaaccgccgaccgggtccgttgacagtttggcaacttgcccgattacatcgtccagcgattgccccgacgctttggcgaacgcctgaactgatttgcccagtgcgctaaaatccgcgttggcggatacaccagcggcggcgagcttctgcactgccgatactgtgccacggaaagacccgccggagcgttcggcggattcccctaatttaagaatttcattagcagttaggccggagaaatggctcgtcatattcagcgtcttattgagacttgagatctgacgatcagcattcgtcaccccggcaccgatagtggcaagggtcgcacctacggcggcgatggataaccccaccgggccgattgtgaacgctagggcgcgcagagcattgccgatgccgccaaacatatcgcgcaattggccgccctgctggataagtaccgttaacgggcgctggccaccctgcaaggatgttacgatgtccgtgatctgcgccggcacgccgcgcatggtggcagtgagctgcttctggctcatgccgaatttgtcggccagttctatctgccgttgctgcgccttagccgcgcgctcggtggcggcggaatttttctctaatgcggtcgccgccgatccagctgctgcgcctgcgccagccactccggacccagcctttttcgcggcgtcgcctaacttatcattagcccgttcggctttttccgccgctacggtaagtttgtctagctcggtgcttgctttcgatacgctgccgacgtcggcgcgcaaactgatgcttgcgatctcttgggtcatgatccgccctcccgtgatgttgccgatattgtacccgcttatcagggcgcatgcatgctcggttatttgcataactgcgtatgcgcttgcgcttcgtgtaatcgagtagaggacgaggggtggctgatcacccccgtctcctcttacgaagtaccggggcgattttggggggcgggcggcttttttctttgtaaatcaataactgccccaggggcgggcgctcggggcgatttgttcggggcgagttaaacgggcgttattttattgttaaattcggtgttggttgcattgttggttgcgcttatttattcaaatggttgccgtaattattcatactataaaattggttgcattgttggttgcacactgctatagttaattcaacttaacaggaggagcagaaatgattaaatctaaccacgctgacgcctacacaccggcggcgcaatcactattggcgactcgttcgaaagagtggctaaatcggtcatacccgcgtaacggagaggcgatcccattctatccttacggcagcccggtttatcagttcaaccagttattgcggggggtgcgtcggtgatcttcgataccctagaagatgcgatcgattacgccaacacccggcgcgcattatccttcggcgcgtcacatggggcccgccactactgtgtgtatctcgacacaagggaaaatatgcatgtcgcatcccatgttgatctgccttacatcaagcactttgatcgcgtgatatggtccacccgtgcagaagtaggtgctactgggtcacgggggaggccacgggcgctcacacggcgtcaggaagcggaattgcgtcgtatgcgtaatgatgggttgccgtatgctaagctcggcgcttatttcggcgtaactgatatgacggcgtttaggatttgtaacagggggaattaattatgggctctaaatttacaacacatgtcgatctactcaatgtggcgctcaatgaacggtaccaccgtatccgtaagagcgttaaacagccgtatggtcgggcgcactggtataagcggtatcgtcgcgaccgagttattatgcgtattttaaagcttcaacaacgtgctattgttcgaagattaggcaagaatgtcgagtcacttgacatctaacaatggctgtaaaacaaaaagcggggtcgctaccccgcttttttcatttcctctttcgccacttcctcaagaacgcggatgtcgtctagcgcctggcgctggtcggtgatgccgtgaatgtcgaacatccaccgcaggacgccatagtcgagggcgtaagccccgcacggcccaacgcgccattgcccggccatcttggtgaatatcgacaccaccggccatacgtccggccacacttctacggtcttgattaactggtccggcgaaacgccatatagctcttgcgcaatgtcttcactcggcgggacggcatacagcgccgccgccgcctctcttagtttttttcgcggaggcactgcaacgttgtagtgtattccttggtgatctccccgtacgcccgcgggaagttcttcaccaatagcaatgcgttttctttattcagcggatcaccaccatcgacgcgccagtcggccagaaggtatagcaggctctcagccatgacgtcgtaggcgtccttatccgggtcttgtaatgcttcgtaggtgtcggccatattacgctgatattcatcaaccggataatgccggaaaaccacatctaacggcacgggcttatcctgtcccgcgattggcaccatgaccgtggcggggaacgtcggcgcggggtttaacttaaacggtgatttactcattgtgcgaagtcctcttaataaaaagccgccatgcggcggccttagtatggcacggttgccgttacagtgcatccacttcggatttaatatagatgcgcataccggattgcatcgatagcgctactgacaccgtttcgacgttgttaacttccgatgacgggatcggctggaatgacaccttagcggcatacaagcggatctcgcctttaccgccagccgccgccgggttgatgaacttgatcgccgcaaaagcctgtgtctggtctgctttttccagcaccgggcggattgggtcctcaatatcatgggttagcgtgtaggtattaacgatcgggtttttgaacgtgttcaggttgatcgcctgcttcgattgcagcggctgaaatgaaaccgtctgctggtcgccgccgctggtagctacgttcgtgatatacgggaaatcgacgaagcccgagatcttgaccatttcccccggtaccgacggagaaaaggcggacgccggataataagtggtatccgtggtgtcgagttttagcatggtgatcgttttctgcgccgtgtctacggccttcacgatcccgcacaggttgagcgcctgagtccacggcgatttagtaagcatcaccgtatcgccttttttcaccgccgcgcccgctacggagcttgattcatcgtaggtgaaaacacattcagccgcattggtcgccccaatcactttgatcgggtccgacagcgtagcgcccatctgcacactactgccgtttggtaattgataccccatgttatgatctcctcatagcccccacggggcgattaagttaacgctctgtaggttacagagatagggatattataccccgtgtctgtcgccagtccattaaacaccgaagccgggcccgaaacaccaacggcaaaccccgaggcgtcgcgcagcatcagtgttgccgggaatgccgcggcgatgtcgtctgcaagtttttcaggtgcgcgtgttccatggcccgccggaatcatcgcggttatttggaaaataccctgatagatcacatcggtttgctgtagcgatatcgaatcactctctgctggcatgaggttcggcactaacataacattggtcagcgagggccgatccgtcggggtgttttcccacgctaccagaatatcgccgagggggtatttcaaccccgccactacggctgatagatggccctctagcagggcgcgtatacgggatacgctcattgtttagattctcctattgcctgtgcaatataacggcgtgcttccgcagcggttatgcgcacgatcccgttaggggcttgattagaccaaccgaactccaggcgacgcgcatacggcacattattgcaaaaccaaacgctatgcacttggtccaagtttagcccagcaagaacggcatcacccgccgccagtgtagccccgcccgatttatcgatgcggtcgattgccgtatctatcggggcgttaaacgacacctgccagttacccctgaatcggccgcctgtatatccgcgtcccacttcgcgccgctgaatgaaggccaccgttttaccgttgcgcgtcttgaattctctgcgcactccggcatgcaccttttgaccgcgctttaatcgccccgtttttgtaactctacgcggatctttacgccgcatagcgttaatccggctggcgcgtgctttagacttagacagttcggcattaactttccatcgcgaggggtcgccgaccggggataacgtaatcaggcggcccagtatcttcatgccgaaggcccgcactacttgatccatgcgctggtttgtcttatccgcgaaaagccgtaccgactccgaaaacgcgcccactgttaccccctaagctgcaaattatatgcgatcactgtcgagccattcggcgctgccggattgggggtaatcacgcggtactgtttcccgagaatggttaccaggtcgcctgtttttacttccgcaccggcaccagacgcggcaaaacgcacatcgcccgccataatgcgggtgccgtctatctccatcggcttgtattgcgccaacacgccggtaatggtgaacgtttccgcagggtggataatctcatctaccccgacgcgctccacccacgccgggcgagttacggttgcggccatgccgttttgtgataacagccgatccgccgtcttttgcagtttacggtaattcaaagccatcatcgttttccccgacgttttgcgcgagctctacggcggctgtttgtggtacaggctcggccggagccgatggcggcgtctgttgctctaccggttgcaccgcaattagcgctgcagttactggaatgctaatcatgatcaccccctcgacacgctaaagccaaaggccatgccgtcggattccgtccacgacccgatcaacccatcaagccacggaatgtccgcgcgcatgccgatcgtgtccttgtcgtaggtcacggataccgcattggccgccgacacactgatctcctgtgccccgctaatgctcggcataaggtcgaacccgtcggcgcataatagcgccagccgataaaccgccgtaaacacgtcctgtggtgttcctgtcacggtgcgcgggtccacaccttccggaacgtcgatcggcgtctctgtggcgtccagcagcgcacaatcaccccacgcgatgccgatacgtggccaggcgtcggcctgatatttgtccgctttcttaccggaccattttttcgagttgatgaaatccgtagcctttgtcagcgcgatcgctgcgtcagcggtagtgatcgtcatcccgcgttccgcggcgaacgctaccagtttttcaggtttacccaacatacaacccccaatacgaaaaaggcggggcaaaagccccgcccctagtccgacatagtgccgggttagaccgccgcacccgcagtggtggcggtaagtttcatcagtacgccggcggtttctttcaggttcttaaccgggactttttgcgagccaccagtgtttttaaccggtgcgttatctactgcgccctggtccagttcccagtttttgtaggatgacacatccgccaatttagcggagcgcaagccctcgatttccgtgcgcagggaggatttgatgcgatatcccttaacggcaacgttgaaatcaaactcgccctgccaccagcgctcgatgttttcgttgccgcctttctgttctgccagcatgtcgaggccagtagtagtgatcgcaaccgcgccgggtaccagaccgataatcgcatctgggccgagtttggtcgaggtgctggacgcgataacatcggcaagcgcagtgccaaccgcatcagagatcaggaagcgacggccaaggccatcttgcagcacttcaatgttgccgatcgcgaagactttctctgcagacggtacggcctgataggcgataaattgcgcccattgggtaccggacatcgcccaggtcttgatgttgccagcggcgtcgccgaacaggctagcggctaacgggaagtcggctagggtcgggaacttcatgccggtagcggtaacgttaacgcgcgccggttgggtgtactgcgatgccgcgttactgcacagcgcgccaccgacggcaccgacggcacccttgatgtaatgctggatgatcgcctccgtcgccagtgcggatacttcaccggcggtctggttaacgtcggtttggatcttagccatcatgccggacgtaatagcaaccgggccgactttcgcagacaggttaatgctgttggtcagcatgcgggccagcactttggcatcggcggcagtgcctacaggcgcataggcgttacgatcggacaccaggccatcgataatgccaacggtgactttttcgatcacatctttcagcacggtaccgctacccatgacgatagcgccgccgctcgacgcgttccacacgttcagattatcggcaaccaattgaatggtagtgccaattaatttttcctgaaatacaggtaagctcatctttatggccctttaatttatgggccgtgcggcccggttgtaaccaaatgtcaaacacataataccacaggtcgcacgacggccaaattattcgccgataccactagcgatctcggtcgcgcgttgcgccaaggtactctgcggcatagcgccgccgccagcaggctgaaagccacctttaggcgtgccgcccgcgcctgaggcaacaacaaccgcggcataatcggcgttagtgcggaattctttttcgaggtcatccagtgacatcgccgacggtttgccgtctttcatcacgcgcactttaaaatcgccgtccacttcttccaagctcaggcgcggtgcaacatgcgggagcatcaacccggcatttttaccgaaaacgcgggaggccagatctcgcgcagtggcgccgattgtcagatcctgcactttcttactcaacaattcggtgcgtccgctggcctcggcttgcactttagccagtttttcctgccaggatttatccagcgcctctacgtcaccagccttgcgcgcggcttcttctcgcgcttgtttctcggcttcttctgcggcgcggcgcttctcggcttcggttttcttttcattcagtagcgcggctacctgtgattttaaacccgatacgtcttccaggccctcgacggccagtacgtaatcgtcgccggacttgttgtaaagccctttaacggcatcgtccagcgcgtcgaacgctgcggaatcgatcttaaacttcaacataactttgccccctaggcataagttaaggcagggccaccccgccttttcatcatggaatactatcgcctcaattggttgcgtgcaacttagtcacgccacaggcccttatttttgagctgcgctatggtgtacaactcgccgctatcgttgaacatcttcggtaccttaagcccgcgcagaatctgatctgcgcgctcgatgccatatatttcttcgaggacgtgccgcggctgccgttgtacccactcgaaataggttgtgccctcggacaccgattccgacatatagcgcccggtcgcgggatccgtctttagcgcagggcgtgttgaatcaggccaatcatccaagccgcgaataacccatgtctcggtactccggcagcagtaatgcaatttacccggcccggcgccgtactgactgcccgcaatatgctttttcagttcacgatctgccgatccttcggtgtccgctttcacttttagcgggtagaacagccgatcgcgtaattggcacatcggcgatgtatgcgtgtcgagagtggataaccaccgccggcacttcactacatcggcattggcagacaccatcaactcgcgcgcggttgcgctatagtgattcacggcagatttcaccaccgaagacacgccagatgagaatttaccctgccaggtggcttttactcccgcgacgatctccatagtgggtttacccatcaaatagcccgcgcgcacctgattggtgatcaagcgcttcgcccaggccgccaggtcatcaggccatgacagtagcgtattaccctggaacggcgcagagaacgccgccgcggctacctgcgcgcctgtaatggccgcgatgggtactaccgcacctggtatcaacgcggggcgaattgccgttgttagcgcatccgcgtaaaacgatgcctcggtgtcggcgaactcttccatggatgtggccagctcggcgaaatcatcgcgcaaagcctcgctaatcgtgctgtctacctttcgcagggcgcggcgtaactgcgccggggtgattactgcgcctctcaccttctccgacgcaagggccgcggccagcacttcggcaacacgcggcgcgagccgtttaatcgcgcgcagcactttagccgcttggccattcgagaaacgctggacgaaaatatgccggcggatcagcagatccgccagcttatcactggccttcatcattcacgcccccggtcggcggttcattctgcacgcgtagcgcctccatgatatcttctacgctgcgcgaatcatcctcaataccctgacgcatcagatagcgcacaaatgattccaacgtaatgacaccagtttgcacaccggccatcagcgcggtgatcgcctgcgggtcgatatttagcgctgtataggtgcggtctaacatcacggagccctcaccgacaacaaaacgcccggcgatctccagcgctttgttaaatgccgcctctacgttgcccgccgccagggctaaggcagaattatctgtccgggcatcgtaatcggcctcagttgccgttttaggggcggatccgcgctcaatcaaggcggcaccgatcatcgccatctgtttttcgcggcgctcgcacagggttaaacatacgtttcgatcttcggcctgcaacaacgaggccgcgctatcctgcggcagtggtaggccgcgagtagcccctagtgcgatgccgttttgcaggtttttatctacccaagtctgcgtaagtccggagactactaacgttggctgcccgactacatgcgccagctcggctaaatcggcctcggcggcgaaatgcttgatgttcagccccgccaaatctgccagcggcgccgggtcaacactggcgttattgtcgaatgccccgccccatgcccagggcaactcctcgagaggcgatccatcacggtcgcgcaacggcacaaggtcggttttagtgaagccggacggataggcgccaatggttgcaccggtgttgtgaatccagcggcggcaatgtgccacgccctcgacgagacgcaactcgacatactcggtgacgctgtgcatagcgaaatcatcgacatcatcgggcatcacctctacggtgcgttgtgttacgacgagcgtagtccgcccattctcctcgcgccagttgatcacctcgcgggccgaatacaggtcgatcaacacgcgttgccccgccgcctcttcggcggtcatcggtaccggattaccctccgcgtcgtattttgccggtctgctgaaatctaccaggaagccaaagcggccgcctttcagcgcggcggatagtgccccgcgcaacacctgagcaatcggcagccctctgccgtccgcgttctctcgcagcacatccagcgcaccagacaggctaacctctaccggcttggcgaacgcgacgcccaatagcgcttgcagcgtgcgcccggtagcgttgaggaacggcgcacgggctagataactgtcgtagcgcttcgacgccatcgggtcttgactcgggtcaaacgccggatgcggcaggtacttcgtcttctgtgccttaacggcgcgctctcccgctacgcaatcgtcgatccgcgcccactccggcgcgtatcgcgcataatccgggtgtttggtatccacaccagccataatgataatcctcagtagaaattaacgacgatcgaatcgttggccgcagggcgaacaatcggatgacggaatgcaatcgggtagccgatggcgtcggccatatggtcaacacccgacgttttatccggcgcgcctgttttcgggtcccagatttgctgctctagtgcctcggtagccttggggcatcgagccacgttcacttttaaacggcgttggcctttgccgtttaggatcatgccgtttacgtcattgacgcgatcgcgcaccgccgggttgacgctattgtatgacaccgtaaagcctgcgtcctcaagcatcgcaatatcggacgtattggcgttcgttgtcttgcggttctttccgctggagtccgggaaaatctcaattaacccgcgcgcatggtgatccgggtatcgctctttaatcgcgtcgatcatggcgtcggtatccagtaagccgcaaaactcgtctaccaggtgcatttcctcgacgccatctgcgatccgctggacgtacaccgcgccggccatctgcccgacgttaaagtccatgccgatcattaaggtatcgtcatcctcggcaacggtatcacagttattcagcgtgcggctgaaattgcggtatacagcgccgccggtcaggttgacgaattcaccgtttaggtaagcctcgatgagctcaggtggatattgcgcccgcagcgtgtcgatgtagtcctgcggcaaatagtgattatcagtggtgcgggccttgatcaggcgcttctggtcgtccgcttccgatatgaagatctgatacatcgcccggaatccttccggagtggacacgatcaccatctggcggacattaccagtacgcaaacgccccagcagtttgcggtatgcctccatcgcaatatctggcttggtagtatcgaactcgtcgcacacgcaccaggcggcgttaacaccgatcaggcgggtgtagttctccatcgagtcgcagataatgcgcgtcatctgccccgcaatacggcaatggtagatcttatcctgcttgttgaacttccattttatcccagcttcgtttaacgccttctccagctccggatacatgatcttgacgagtagcggtattgtcggctcggtgataatgccatcgcaacctggattcaggatcgcgagttgtaccgccttgcgtgctgccgaccatgttttaccgccaccgaagcccgagcacaggcctagtattttggtcgtcgtatcgttgatcaggccgcgctggtgcggcagcgtcttgatccgccatgtgcggcttttccgctcctttttcaccggcgcggggacaccaaggcgcattacctgccgctcggctttagcaatacgcgccagcaattcgcggtcgaagtcatccatcacgcgttaccgtcagcttctgcgaccacattcacatcggaatcatcctcatcgctaaccgtatcaggttgctgatccgtgatgccgtggtttgctctcatctggaagatcgcccaggcggacggcatcagacccaccccggcggcctggctgatgaattcctgttgcagttctttgcagatggcgtaggcctctgcgaagtccggattagcgcgcgcccataacatcaggttccagcgcgtcaccccgatcaggcgagcgaattttacgaaggacggctggttatcgcgggggatcacctgggcattgcccttgtcggtgtagttgagctgccaggcggtcgcattctcgaagtattgcaccattttctcggcgtactccgggcggtagttggtagggcggccgtacacgtaaccttccggcatttcaccatgacggcgttttgtaggcatgcgcaccccgtttactttaggcttgagctttgcttttagtgccataaggcccccatgttgctgctctgcggtggaccatccaaacgcgagaggcggacccctggcccgccccttgattatatagcgcgtagaaaatggcgcaatgtagcaaaagtcattgcaacgcagcagttaaagtgctttaatacccttacacaaaagctctttcccataagccgtaggtgttccaccaccgaaagctacaaaaaggcccgcattatgcgggccttttcttttgcgtcatccagggtacacatcccggcgtaattcgaagtgcgggccatcgtaaaagcgctcgtcatcgctgcgcccgttctgattccagtcgccaccccagcggatcgcaacgccctgctctttggccgccgcaaacatcgcatcggacactgacttaaatggtgcgcggtcgttccacgggatagcaccggcaaccaacggcgcgcagtctaccgcatgcccggttagatggcggctgttcatcgtctgcgatttgccagtagcaaccatttcgcgctgccgttctacggaccgtcgcccctcgatcactgtgaaatcgaccggtgtcagttcgatcgcgcgcttcactacgcggactaggtcagggtgtacgccgtccagccgtgacaggctgcgactgcttagtttgaacattattcctccacgggatcataggtaatatggaaaatatccggtttgcacggataaaattcacccttcacgcccttgataatgtaatccccctctgttgctacgtgacgtgcgcgcttatccggcccatcctcaagggttactacatcacaaaatgcaacggcgcccgggtgtctttgtttgcgaatgtttttaaccgcgcctccgcaccactctaaaaatcccgcggatggcggataagtaaaacgcatagcttctattttaactggctttttcacaaataacgtcggcattttaataccctctaaagttaattcgatttaatactagcgcatttcttgctgtttgtcatagctcaattcgcagagattggccgcttcccaggatcgatcggcgaaagccgccagctcttcatttcgtcggacagatttttcgagcacgtcggtaagcaatacggcggtttttccggtgctcgagccagcgggctgagagtcggaatactgcaagatggtgcgacgtatcgcggcgatttctgcgcgcagcccatcagcagtacgacgggcagcgtcagcatcggcaacagcctgattgcgttctgccagggcccgcgcttcgtcttcagcctttgcggcgtggcgtcggtcatcttccgctttgcgcttcgccagggcttgtaggtcccgctggttctgctctgctcgatatcgttcatagccgttcgtatccccccgctggtatagccaatagccggttgccgttaagccccccgctagggccagcccgctcaataacgcgatgatggttttattcattgcgcaccttgcccttggccctgctcacgatgcccgcccactttccgcaggcgggcttccagaatgcatcccggataagctcccgcgccatcatgaacgcgctgcacaccagcaatgtggtaattagccgttctggtagttcgcctgtctgagagtgcagcaggccgccgagactggtcacggcagccgccatgtacagaatacgcccgattacgccgtcgcgcacttttgggctgtagatgccccatagcgccatgaggaagatggccaatagcgccaccgagtagatcacgttgtgctcgatcatgattattgcccccgcttaaaggccccgattaggtccttccatcttgcggcggcgatcgcgccaatgtccagcgaatttaacgtttctttaacacgctgcaaaaggctcatgccgaacacgccgatgagaaagccgatagtggcgatactgcgctcgtcagtcagtgccagatagtggccaaccggctcgctgaggtagaacgcggtaaccacaccgccgaggaggaaacatgcgcgctgccggaaggtgcggatctcggtatgcaccaccaaccccacgattgcgcccataacgcccgccaggagcacccaagacgctgatgatactttctgccataagtccataagaaaaggccccgtaatgtgaatacagggccatggtaacattcgatgcgttatcgcgcgattttagcgtttaaacgcaccgccgatgtgctgaaacagttcatcgattttatcgttgatgcgatagtcacggctaagcttcttagcatcgagggtgccgttgataccaattggttctacgccggctttcgcagcgcggcgatccgcgcgttccgtaatctcggcaacgatatcgacggtaaaagcgtgcgggccggacaggctttcgtattggcgcgttgccgtctctacctcggccatcgcgttattcagacggtaaccggctttcgcgtactgcgccaggcgagctaatgccgcggcgatttcttcattgttcaaatacactccggtttttggttgcccaaaaacttcgatataacgcatcataatttcgccgcgtaccagattatcatccgccgggcgggttgcgttactcgtcttctctcgcgctagatcgcggcgtgtcaattctaattgacgttgcaattcgtcacgctcgcggatgattacggtgcggtcttgtttcaaagcatcacgctcacgcgccgcttgctcgtatttcttggctaactcatccgacggtttcaccacggccgacagccgatcggcctcgcgctgtttcagctctgcgatcagcgagtcggcagcggccaggcgggtgcgaagcgcctcgttttcggcgtgtagttctttcgtggttgtaacagccttatccaggtcaatgaatagcgccgattcaaattcgttcattatctgctctccagtatgtaaattgcggtgtggcgttatgccggtcaccgcgttgagtatagtcaatttaacaatcttcgcaaggcatgttgtcgagccgcagtaattcgtcgatcgtcgcctgctttttggctagctcctcttcgagttgccgattgcgctctgccagctggtggcgctcgcggtcgcagcggttgaacatggactgtaaacgggcgtattgcgggttcattgcaatctcccgtccatcaattccagttcgcagcgcatagagtcgagctgccgctcaaggcggcggcgttcggcgttatacatcttggcacgcggcagggtgcgccactggtagtccgccttttcgattgccgattgtaagaatgcgcggcgctttgcctcgcggcggtacttcatgatggcccgcataggagccgtgatatacgcgataagtttttgcatggcgctactccttcggcaatctgatcatcgcgttaaggtcttcgcgcgataaggctagtcgcgtgccagttgctgcggcttgcttctcgggcagcaggtctttcgcttcgggccaggcctcgaggagcttctcgacggtgcggaatttgctgatggttgctttaaccgaggccttgatctccgtatagcgattgcgcagtgccttatcctcggcctggaatacggagaattcatccagtgccgttgtatccgcgattgggtaccacccggagttagggcaaaacgcttcctcatcgaagtcaaccatgctcccctcatagagtggatgggcttcaccgcgcccattgcgataaagatacatccgcaggccattaaggttaacttgtacgtagcctacttgataaacgacaaaatatgcgttctggttagtgaaacacttcggtaaacccagctcctctatatgcttcaccaaggcccgcagcgcctcatcagacgttttgcattccgctagggcgcgctgccgggtacgttcggtgaagtcggcgcggcgggcgcacagatcgcgttcttgttcgggcaggccagcctgcaccagtgcgttagctacgatcgcgtcgcggattgctgcattcatacgtgttgccatttctgctattcctcgttgttgttaattcgaattaactatagccccgctcgcggggatgtgcaagcggtatttactcaatttaccgattcgcgcccctgggcgcgccaccaggttaggcgtaagggttgttgtctttctcaaagcggaaggcgcccttgtactccggcatggcgtgcatgattaccatcgcgtcgtaccatgctttcattagcggagccaactgtgccccgagcacccgacgatcggcgtaggtaatggccgggttttcgaactggcggtttattgcgtcgatatctttttgcatatccgcgatcatcccgcgctgttgttgatacgtggtcatttctgcaatccctctttcttgttaagttgatttaactatagctcgataaaatacggattgcaaggtgcaggcgcgcaaaagtgcctagttaccttgcgtgacaattcgcagataacacaacgcaatagccggcgataacacatctgtacttgcaaccaacccgcaatccgctatcacgtcgtgttgccgttttgattaaccaccgtccgctagcctcacctttcttttaaactgcggcctacgtagtgcaaccaaataaactgccgtactgacccagtgcgcattctgtcagtcgtacaggattacattacacaaccaaagatttctgttgagtgacccaggcgccttaaggcgcccatagggctaatcaacactgaatcaacaaatacctaaaacaatcggcaacgatccaaaatggttgccatgagaattacagagtgcatacatacatgctgtgaatacgcgagtgctatggtttattgcaagcatgtatgttgcaaaaccactctcccatttataatgggccccggggcacttattgatttataaggattttttggcgttttaacatatgtacggggcggttatctgggcgttaaatttttgttaaatcactgtatgtatttacagtgttggttgcgcataaataagcgcaaccatacgcgaattatgcataattaatcaaaaacagtgcataagttaagcgcagtaaatttctgattattgcaaaatgttggttgcaacactttttccttataaatcaataagtgcccccggggcgggcgcggggcgatttttagcgctgtaaactttcgcgttggttgcggttttatttagacgtgcgaaagcaaccaacattatgcacgttggttgcagtatttgttggttgcagcgtagctaatcttctaaatccgagttggttgcacttggctgcggtggtttttcactatccgataattgttgcggcgcgcaatcccaatccttgaatagccacccggtgatctcaccgtcgtcattcgcggctgggatcactacgccggcctcctgggcctcgcgccatccctcttggaacttcttgcggtcgccatgtacctttttgtataccgagcccgccagggcctgcgatataccctcgggcgcttcgtcaagcgtctgtagcgcctcgatcagcttgtacagtgcggatgatgccttgccgttcttctctttgatctcgttggccatagcacgagcgggcgtgacggtgcccgcgtcgaacggcgccagggcgacagggacaaggtacagtgtctcgtctggcggggtggtcttgaacggcgccggctcgaagccggttagctccttggagaaatccggcgctgctgccgtggattggtattggccgacgagctcggattggttctcgtctaccgggatcttgcacgattgcaggatgaacccgcgcggcgattggcgcatgccaaaacgcgctttctcgtggtagaaattgagttggtgctctttatccggatcaggctgctcaaggaagaaagccgcatcaacggcagcatgcagggccccactacctcgcgccgtccggttgccgttgttgttggacttggccgggtggtggatgacgcccggtgatccgcctgtctctctggcgatgtctttcaggcaggccaccactttacccatatcggtagcgttgttctcgtcgaatggctcggcggcgagcgcggtagtggcgttcagcgagtcgaacgtgataatccctactggttcttcccccgccaggtcgttgatcaggcgcacgcacttcttgcgccccgctggggtggtaatgtcgatcccggcgccagcggtgtcgatgatgtgcaggcgggataagtcggactggtattttatctgtagcgcttttttacggcgcttggattcctccggcgcttcggcgtcgaaatagaaacagtgcgccgggatcaccgccttgccggcgaattggataccagccgcgaccgccgccatagttccgaggatgtggaaagatttcccgatgttcgatccgccggccgcgtaccaggtagagcggaagtttagcaacccctcaatgatcgggtcgtgctgcgtgaactgcgggcggctcggctcgtcttccagatcctcatcggtgcagacgtacaagtcctcgtctgctgccgcgtcgttctcggcctggtgcagtcgctgcaactcttcatcatcgattaacggcagcgcttcggcgagttgtgcgcggctgatcggatggggtgacggtagcaggtagtcgggtacgccgagcatacgcatagctaggtgctggtggcggttgatgttaccgtgcccggagcagttggcgtgctggcagtggaaatgcacctcggggtgcttggcattgggtaataggattgccgtggagctggtcccgtccgtgtcggtggagtggttggccgagttcgggcactctacaatgtaccctcggcggctcggcatgagttcgagccccatttcctcgcaccactccatgatcgcacgtccgttctcgtccgctttggcgaggtcgtcttctgatagcgtagggcgatccgatttttcggcgggtgcttcccacgccatggctaaaacgtcatccaccgccagggtgcggccagcgcccgtgcgaaatgccgcgttgcgatgcggtacgaacatcaagcgggcgcgctgataggcggtggcgtcggcgcagtcccacacgtccagcatatgcgccagcgtggtgtttacctgccagatctcatccgcggccatcgggcgatcggtggggatagcaaaacgcacgctgcgggtgtcttcacctttcagcgggtggcgatcacccggggtggtgtattcgagatagcgcaggttgaggcgcgacaatgcgcggcgcaccaggaaaaggcgtgctggtgtgacggcgtccaggtcgagccagcagatagatcggcagtctacgccatcgtcgccgcgcttggcgccaggtagcacagatgcacagatatatttttggcggctcttcttggcccggtattcgtcctcggtctcgctgccggtgaacgtcgggcggttctcgatttcgtcggggtcggtcatgtagtcgacgaatttgcgccacgtcatggtggtgttttttggcctggcatcagtcgcgctcgtcccgatcgcgaactgtaggcggattaattttgcattgtgcattgtgctgtcctatactcgaagacgtgcgatcgttatttctgctttcccacaccaggaacctttaccagtgcggcccgtatcatttttgatacgggtttttttttgtttttaaccctcggccagccagcgcggatcgcagcgcagcacatcggccatacgaaagaggtttttcgagttcatcgactccgcgttgccgttcagtagctgggaaataaaggggcgtgacaagcccgtagcgcgggcaagctgcgccggggtcattgcttgcttggtcatttcggagcggatgcgctcggccagggtggtagtaatcgccatggtaaattcctctatgaatgtaacgtgtgtgcactatagaatacacgccacgccaactttcggcaactcgaacgcaactcaaaaacataacaccatgattgtattaagctttttaataaagttgaaaaaacagcttgcaaacatggtaaattgatttacagttatcaacaccgaaacacggtacacactaaccaactgaggattacacgatgtttgagaaattactcgccctgttcgaacgcctggttatcgcacaagaagccatcgcagccgccggtaagaagtattacacggaagctgaagcggatcagaagatggccgaccatattgaaaaaaaagaggcttccgaaaaaccgaagcgcggtaaaaaagctgccgccgctgaaccggaagatgatccggttgatgacaagccaaaacgcggccgcaaaaagcaatctggccccgatcttggcgcgatgcgtaaagaagtcgaagagctggcccaggtattcgccagcgcggatgatgatgaagcgctggaggagttcaaaaaactcctggaagatttcggcgagcgcaccgtgaagaaaatctctgatgacgacctgccgggcttccacgaggagctgaaaaaactggccgatgagtttttcgagttcgaagaagaataacactacgctggcccggttatcgccgggccactttttagaggtgtaaaaatgagtgcttataattgggccctttgcgatcttttatgccgtagagatttcgtatggccgcgcagccttgcacaaacagtgtcgtcacgcactcgaccgccaaacggttggcgttggagtcttgtcgcgacgggtgatttcattatgcgtgggccaactgacgatttggttatgtacaaagacaactacgatgcggcgttgatgagtttgagaaacaaaaaagatatcaaaaaatacgaaacgtacacctcggctaaaccgtcaaaaagcgccgccgatatcctcacggcggcggcggaccatatggccgagcgcgccacacaacgcgatacacctggaggcgagcgcactatgtctcgcacggtggccgcattcaacgcgatgtacggcaccaacttaaccgaggtgcagggctggcagtttatggtcctgttgaaaatgtcccgcgcttccgcgggtgcgcatgttgccgatgattacgaagatcagacggcgtattcggcgctagcgggtgaatgcgcaaatcgggaggattagcgatgcattatcaactctatatcggtactgatctgcgtgatggcgcgcaagcgttatggctcctgcgcggtccggtcgatgcgatgaccgagtgcgtggcgttgtcaccgaaagtatccaacgtcgatgtgatcatgaacacgcgccgcgagcgtgatccgtatgagttcatggcaatcgccattttcgaaaagcatgcgcacgccgtggcgccgcttacgtcctgggaggtttaaccgtggccgtcctgaaagcgaaacgcaaaaataaagatcgctccggtagtaacgaggagcacgcgctattgtcgccaagctccgctaaaaagtggctcggctgtcccgcggcgctcaccgctgaaatcgggatccccaacccgtcaaatcctgcggcggaagcgggaaccgcgatgcacgccgttgccgagattatggcgaataatttgatccgcgatggtgaaagcaaggctgcgtctgaattcgtcgggggctacccgctgcataccccgacgaagaaaagcaaggggccgaagttcaccgacgaaatggccaagatggtgcagggctacattgacacctgcgtagcgcccctagtcgatgccggcgccgaagtgtatatcgagtcgcgcgtagaccttagccgcccgctcggcgcacctaacactttcggcaccgcggacttagtggccgtcacagagctgaccgacggatcgaacatgctgatcgtcggcgacttgaaaaccgggcggcacccggtggacgccaaagaaaaccggcagatgatgatctacgcgctcggtttgctgaataaatatcgcttctcgcacgatatcaccaaagtgcgcttgatgatttatcagccgttttgcggtggcgttagtgagtgggacacgtcggcggaagtcatcgagacgtttggcaagttcgcgaaagaccgcgccgctaaggccttggcgtgccacgccgccggtaaagccgcgttaaagcctggcgacttccggccatccgccgatgcgtgtcagtggtgccgttttcgcgagaagtgcaacgcagcgcgcaagttcaacgagcagatcgccgctgacgacctacgtgatgagtccggcgacgaaatgacgccagaggagctggccgaggcctacgccaagttaccggcgctgcgccagcacatcaaaaacatcgaatcggcaacgtataaggcgctgttagccggtaccaaactgcctgggctgaaactggtagccggtaaggatggtaatcgcacctggtcagatgaggcgcttgtgcaattgcgtcttgagcaaggcggcgttacgccggatgcgatgtacacgcagaaactgctaacgcctacccaggccgaaaaagcactaccggcgggcgcgtttgagtgggtggaagaactcatcacccgcaagccgggcgagccgtcgatcgcatcggcagacgacaagcgcccggaatacgtgccagttaaagacgacgatttagtcgattaaaaattggttgcaatgtcctacgtgttgtgacctaatacataagccgacgcggcggcccttaccgcgataaaaatgtgaattggagagtgttaaaatggctaaagtcaatctgaaaaatgtccgtctgtgtttcctccacgctttcgagcgcgccgagccgaaaaacaaaggggaaaaggccgcctacaaggtgtgtatcctcctggacaaagacgatcagcaggttgaaaaactggaagacaccgcgttagaggtgttaaccgcaaagtggggcaagcgcgaagttgccgagcgttggatgtcgcgtaactatgcgcaggatagcagcaaggaatgcgccgttaatgatggtgacctgcgcgaagaggttaccccggagtttgaaaacgcgatctatatcaatgcccgcagcccgaagcagccgaagattcaaacgtctttaggcgaggaccagaccgagccgggtatcacggttgatggcgatccgatcgagggcaaagaaatttacgctgggtgttacgctaacgtcagcattgagttgtgggcccaggataatgaacatggtaagggtctgcgcgctgcaatcctcggcttgcgtttccgtgccgatggtgaagcgttcggcggtggcggctcaacggcaaccgatgacgacctgagcgacgatgatgacgagccgcgtagcgtatcccgccgccgcagtcgtgacgacgaagatgacgcaccgcgcggtaagtctcgcaaccgtcgtgatcgcgatgaggatgaagacgatgaaccacgtgagcgccgccgtagcgtatcccgccgccgcagtcgtgacgacgattaataaaaagcctcgatagtacctacggcctcgcatgaggccgtttttctaagggccgcattatgccacaactcctatttcttgacttcgaaacattcagtgaagccgatttgaaaaaagtcggtgcctatgcctacgcagagcacgattcaaccgagatcctgttagcgtcatacgcgtttgatgacggccccgccaaagtgtgggacgctacttgcgcatcaggcgaaagcgatatcgatctagataacaattccgcccccgatgatctgctgcgtggcctgcgtcgtgcaaaacgcgggcgcgtcaaactggtgatgcataacggcttgatgttcgaccgcttgatcatccgcgaatgccttggtctcgatatcccgccggagcacatccacgatacaatggtgcaggcgttccgccacgcgctacccggcagcctggataaactgtgcgaagtgcttaacgtcgatgccgacctggcgaaagacaaagcgggtaaggcgctgatcaagcgattctgcaagcctacaccgaaaaactacaagatccgacgctatgaccgcaacacgcatccggacgaatggaagcaattcaagcactacgcgcgcaacgacatcacggcaatgcgtgagatctactacaaaatgccgtcatggggcgagatagacaaagaaaacgagatcttggcacttgaccagcgcattaacgatcgcgggttttatgtggacactgatttagctaaagccgcgaccgccgcggtggccacttacggcggcggccttaccggtgccgattttctccccctcctgcgcgatctggcacccgcgcatcacatcccaaacgcgcagaaatcaacgctcggtgacctgctggatgacgccgacttacccgacgaggcccgccaggtgatcgaaatgcggctaggcgcggccagtaccgccagcacgaaatatgcccccctgcttaatggtatgtccgccgacggccgccgccgcgggtgcctgcaatatggcggcgccaaacgcacactccgttgggcgggcaagggctttcagccgcagaacctggcacgcgggtatttcaaagaaaaaccgctagcccgtgggatcgaggcgctgaaacgcggcaccgcggagtacgctttcgacgtaatgaagctggcggcatccacggttcgcggctgcatcatcccggcaccgggtaaaaaattggtcgttgccgactactctaacgtcgagggtcgcggtctggcctggctggcgggggaggattcggcgctcgatactttccgcgcggggttggatatctacaaagtgaccgccggcaagatgttcggcatcagtccggacgacgtggatggctaccgccggcagatcggcaaggcctgcgaattgggtctcggctacggtggcggcgtggccgcgttcctgacattctctaaaaacctcggtctggatctggaggaaatggccgttacgatggctggcactttccctgattaccactggcgcgccgcgctacgcgcctatgaattcatgaagttgcaggaggtgaagcgcaagccgctacccggtaaaaaagacgatcgaacgaccgtcgtcctctctaaaaaagcgtggcttacatgcgattgcatcaaacgtatgtggcgggagtcgcacccaagaacggtgcaattctggtatgacctggaagaagcctgtttgatggctatcgacaatccaggggcgtcgtattgggcgggggccaaggttcgccaagacggcaaacgcgccatacgcatcgagcggacattaacgcgggctggcaaggccgggcaactggctaaagatcgaattgccgtccggacgtatcctgtcctatccggggatcggcgtgtcgatggagaaaaccaacgaggacgatccgggcgagaaagcgcgcccacgcatcaaataccgtggagagaaccagttaacgcgtcaatgggggtggcagcacacctacggcgggaaattggcggagaacgtcacccaggcgctgtgccgcgacatcctagcatggtgcatgctgcccgtcgataacgcaggctatgagatcatcctgtcggtacacgatgagctgatcaccgagacgcccgatacggcagaatacaacgttgccgaacttgagcgcctgatgtgcgacttgccagcctgggccaagggattcccgctaaaggccgagggctgggaaggataccgctacaagaaatgatgggggttgtatgacgcccgaaggtaaagtgcaggcgcacctgcaacgacggtttaaggcgatcggcggcttggtgcgcaagatatcctatgaggggcggcgcggctgccctgacctgtttatcgtgttgccgggtggggtggtggtcatggtggaggttaaaaagcctggcggtacgccggagccacaccaggtgcgcgagatagagcgcttacggcaacgtggtgtgccagtgtatgtaatcgacagtatcgagggtgcggataagttggttgcattttatagctgatttatctatagttggttgcaaggacgcaaccaggagcacgcacaatgcatgacatcttcg

SEQ ID NO: 4 - protein, 155 aa, Bacteriophage phi eiAU
MATITKKQRAELRMKFGGRCAYCGCELSDRGWHADHVEPALRKWEFVKNKTSGVLQTASTGEFWRPENDTLENLFPSCAPCNLFKATFSVEMFREQIAEQVKRARSRSVNFRTAERFGLIKVIDMPVVFWFERYQEGADHQGDSRKASRNWERYS

SEQ ID NO: 5 - protein, 174 aa, Bacteriophage phi eiAU
MNRPASMRAVNVQRMENKTMATITKKQRAELRMKFGGRCAYCGCELSDRGWHADHVEPALRKWEFVKNKTSGVLQTASTGEFWRPENDTLENLFPSCAPCNLFKATFSVEMFREQIAEQVKRARSRSVNFRTAERFGLIKVIDMPVVFWFERYQEGADHQGDSRKASRNWERYS

SEQ ID NO: 6 - protein, 449 aa, Bacteriophage phi eiAU
VWATMGSGKTGATMWALDAMFSTGILDESDRVLILAPLRVASGTWPEEQRKWKFPALRVIDATGNAEHRIEALATSANVVCLNYDVLEWLVEYYGNDWPFTVVVADESTRLKSYRSRGGSKRARALAKVAHKKIRRFINLTGTPARNGLKDVWGQMWFLDAGERLGTSYQSFSDRWFVSKQVGSSPLARQISPRTGAETEIHQKCADLSITIDAAEYFGCDKPVVVPIVVELPKKARKIYDDMENALFAELESGEIEASNAAAKTAKCLQIAGGACYITTDDGEASKEWTEIHKAKLDALESIIEELNGSPLLVAYQYKHDLVRLLKRFPQGRAMRKGLKGNNDMADWNAGKVPIMFVHPASAGHGLNLQDGGCHLAFFNDTWNYEQYAQIVERIGPVRQHQAGHPRTVYIYIIQARGTLDEVVALRRDDKAEVQDLLMDYMKRKKRSK

SEQ ID NO: 7 - protein, 483 aa, Bacteriophage phi eiAU
MNISGPRGSNMAQFKRRPYQKAITGHIIAHARCNVWATMGSGKTGATMWALDAMFSTGILDESDRVLILAPLRVASGTWPEEQRKWKFPALRVIDATGNAEHRIEALATSANVVCLNYDVLEWLVEYYGNDWPFTVVVADESTRLKSYRSRGGSKRARALAKVAHKKIRRFINLTGTPARNGLKDVWGQMWFLDAGERLGTSYQSFSDRWFVSKQVGSSPLARQISPRTGAETEIHQKCADLSITIDAAEYFGCDKPVVVPIVVELPKKARKIYDDMENALFAELESGEIEASNAAAKTAKCLQIAGGACYITTDDGEASKEWTEIHKAKLDALESIIEELNGSPLLVAYQYKHDLVRLLKRFPQGRAMRKGLKGNNDMADWNAGKVPIMFVHPASAGHGLNLQDGGCHLAFFNDTWNYEQYAQIVERIGPVRQHQAGHPRTVYIYIIQARGTLDEVVALRRDDKAEVQDLLMDYMKRKKRSK

SEQ ID NO: 8 - protein, 295 aa, Bacteriophage phi eiAU
MTAYYNEIDPYAAQWLRNLIAEGHIAPGIVDERSIEDITPNELTEFTQCHFFAGIGVWSLALRRAGWPDDRPVWTGSCPCQPFSAAGKGAGVADERHLWPAFFHLISQCSPSVVFGEQVSSKDGLGWLDIVQTDLENAGYASAAADLCAAGVGAPHIRQRLYWVADANHQRQEGKQPRHHAEGWEGQDFLPSRLCDGAGVSSVSAESGRVATIRSITETGQSLRVADAEGGRWGEKLQNIGGGTTGVGAREIILPAGVVTNDAAGPTNGHWRDSDWLSCRDGKWRPLEPWHIPVG

SEQ ID NO: 9 - protein, 125 aa, Bacteriophage phi eiAU
PPRRFSGYAVRTWGSFEIDAAAADHNPLVADYWTLADNALVQDWSGKPVWCNPPYSDIGPWVEKAATAEFCVMLVPADTSVKWFAPAGELGASVIFITRGRLRFIHNATGKPGPSNKMGSCFLVF

SEQ ID NO: 10 - protein, 236 aa, Bacteriophage phi eiAU
MGLPPEWGRLRAYGNSICAQLAEEFIRAYSRHGEKLKCLAITIQKPAPRRLEKLKCLAIPIQKRPPKIKIAGVPPRRFSGYAVRTWGSFEIDAAAADHNPLVADYWTLADNALVQDWSGKPVWCNPPYSDIGPWVEKAATAEFCVMLVPADTSVKWFAPAGELGASVIFITRGRLRFIHNATGKPGPSNKMGSCFLVFGGSRPGRVDFVTRAGVYQIGAPRKVTVKRRVRAPPNAT

SEQ ID NO: 11 - protein, 115 aa, Bacteriophage phi eiAU
MKYFKDSKNMVYAYLADGSQDHYIKEGLMPISETEAMALANPPPTQEELITQALDKKNTLLEEARKTTNDWQTELSLGIISDGDKAKLVEWMGYIKKLREINPASYPDIQWPTTP

SEQ ID NO: 12 - protein, 117 aa, Bacteriophage phi eiAU
MKYFKDSKNMVYAYLADGSQDHYIKEGLMPISETEAMALANPPPTQEELITQALDKKNTLLEEARKTTNDWQTELSLGIISDGDKAKLVEWMGYIKKLREINPASYPDIQWPTTPPV

SEQ ID NO: 13 - protein, 160 aa, Bacteriophage phi eiAU
MAWYKTGTIAINGKEVTGSGTKWADPSAGIGEGQALLVPSSGVVKIYEIARVNGDTSMTLVSDASNLPSGSAYAILSFYGQSRPDFARQLAATLRSYQEQSDALKQFYSATGDITVEIDGVQYTGSSFQKITTELDKKADKTYVDTELDKKADKTYVDAE

SEQ ID NO: 14 - protein, 335 aa, Bacteriophage phi eiAU
MAWYKTGTIAINGKEVTGSGTKWADPSAGIGEGQALLVPSSGVVKIYEIARVNGDTSMTLVSDASNLPSGSAYAILSFYGQSRPDFARQLAATLRSYQEQSDALKQFYSATGDITVEIDGVQYTGSSFQKITTELDKKADKTYVDTELDKKADKTYVDAELNKKAEKTPIIAAISILEAAANKILVLTGKDSAKTADLSVFSEELLGKRNADEIIAHLKLGDASKLNVGVASGTVAAGDVTIGVGQAYMDVTADRSIGVIYTNSSTRPIAIKVQVTVPSSVEATIKVGDIVVAGGNTPNVSWLTQWHYLYAIIPQGATYSVSCPTGTLVNWVEMR

SEQ ID NO: 15 - protein, 1051 aa, Bacteriophage phi eiAU
IKGAKGGGGDSHTPVEQPDSIRSQAKARLLIALGEGEMALGLDDTKIFLDGTPLGNPDGSRNFDGVRWEVRPGVQQQDPISGFPAVENETGFGTEIKQASPWVHALTRTEIDAVVVRVGVPALMYQEDDGDVVGTSVSFRIDLAVGGGTFSTQGKFAISGKTTTLYERSIRVNLPRSSSGWRIRVVRETPDSDSARLANTLKIQAITEVIDARFRYPHTALLFIEFNAKSFQNIPKISCLAKGRIIRVPSNYDPDTRTYSGNWDGSFKWAYTNNPAWVWYDVLTQPRFGLGKRVTAAMLDKWELYRIAQRCDQMVPDGAGGVEPRFEFNCYLQAQADAWTVIRDIAAGFNGLTYWGNNMFNVVSDMPVKAPSQIVTRASIIGKPTYSSGSRKTRFSSALVNYSDAQNHYADTPTAVMFQELVAQLGFEQTQLTAIGCTRESEAQRRASWAVLTNSVDRLVKLRVGLEGFAFLPGTVFALADERIGGRVMGGRVAGYDEKTKQVMLDRTTDGKPGDDLLIRTTGGAVESRKIASVGDSVVTIAEPFTAAPAVNAVWVVDSGELALQKFRVLTLDFDDENNTFEISAAEYNDSKYDAVDDGARLDKPPVSLLPTGIVNAPTAVAITSYEQVRQNQRVTTMRATWEPSRMADGKVQPDIVAYEAQWRRGANDWVNVPASSVNGFEVQGVFAGDYLVRVRAVTSFGASSVWASSVLTHIDGRQGEVPAPVSLRASSDVVFGIDVAWAFPKDAEDTEYTEIQYAPTNTEEAFTTLSLSPYPSKSFAHSGLKANAVFWYRARLVDRLGNKSEWGASVQGRASIDTDSIMDALGDQVMSSEGGKALETSINAAIDAIEQNAIANDGDIQRKSKKLGELSAEIVRIDNVVVNEVGALAESLTAVKASVAENEAAVATKMTAKFDYDGNGYAVWDTNAGITYNGEYYSAGMSISAEVKEGEVSTQVAMLADRFAVMAKVGDKPELMFGVVGDQAYLRDAFIRDASIGSAKIAGVLQSDDYTPGGAGWTINKSGAVEFNNATIRGTVYAENGDFKGTVHAN

SEQ ID NO: 16 - protein, 1225 aa, Bacteriophage phi eiAU
MGDERDRRCYNQCRNCCRGRSIMQRQLFYIKGAKGGGGDSHTPVEQPDSIRSQAKARLLIALGEGEMALGLDDTKIFLDGTPLGNPDGSRNFDGVRWEVRPGVQQQDPISGFPAVENETGFGTEIKQASPWVHALTRTEIDAVVVRVGVPALMYQEDDGDVVGTSVSFRIDLAVGGGTFSTQGKFAISGKTTTLYERSIRVNLPRSSSGWRIRVVRETPDSDSARLANTLKIQAITEVIDARFRYPHTALLFIEFNAKSFQNIPKISCLAKGRIIRVPSNYDPDTRTYSGNWDGSFKWAYTNNPAWVWYDVLTQPRFGLGKRVTAAMLDKWELYRIAQRCDQMVPDGAGGVEPRFEFNCYLQAQADAWTVIRDIAAGFNGLTYWGNNMFNVVSDMPVKAPSQIVTRASIIGKPTYSSGSRKTRFSSALVNYSDAQNHYADTPTAVMFQELVAQLGFEQTQLTAIGCTRESEAQRRASWAVLTNSVDRLVKLRVGLEGFAFLPGTVFALADERIGGRVMGGRVAGYDEKTKQVMLDRTTDGKPGDDLLIRTTGGAVESRKIASVGDSVVTIAEPFTAAPAVNAVWVVDSGELALQKFRVLTLDFDDENNTFEISAAEYNDSKYDAVDDGARLDKPPVSLLPTGIVNAPTAVAITSYEQVRQNQRVTTMRATWEPSRMADGKVQPDIVAYEAQWRRGANDWVNVPASSVNGFEVQGVFAGDYLVRVRAVTSFGASSVWASSVLTHIDGRQGEVPAPVSLRASSDVVFGIDVAWAFPKDAEDTEYTEIQYAPTNTEEAFTTLSLSPYPSKSFAHSGLKANAVFWYRARLVDRLGNKSEWGASVQGRASIDTDSIMDALGDQVMSSEGGKALETSINAAIDAIEQNAIANDGDIQRKSKKLGELSAEIVRIDNVVVNEVGALAESLTAVKASVAENEAAVATKMTAKFDYDGNGYAVWDTNAGITYNGEYYSAGMSISAEVKEGEVSTQVAMLADRFAVMAKVGDKPELMFGVVGDQAYLRDAFIRDASIGSAKIAGVLQSDDYTPGGAGWTINKSGAVEFNNATIRGTVYAENGDFKGTVHANRIVGDVVQYSNFTFSSKDVSVGNGATRVLFKVPAEDFEQTIISNGYVKFFAGSGGMTRISCYVESSGVRKVLTELWSNGETAEYKFNLSGLTLPPGANGTWIRIEFTKTWPNTIRPEKPHTLLTYDGAQLLMGRARRGSAEILEG

SEQ ID NO: 17 - protein, 174 aa, Bacteriophage phi eiAU
  misc_feature (126)..(126): Xaa can be any naturally occurring amino acid
VSTTTEAVKALCVTLDGFEEYLLHAKKNGMTFAVFRGRKNIGAEELHDGIGNDEIRIAPVIEGSKKGGLFQTILGAVLVVAGVGLTIFSGGALASFGAQMAWAGAAVMAGGLYQMLSPQPRGLQSXEDPDNRPSYAFGGPVNTTAMGNPIGVLWGTREIGGAIISAGIVAEDVA

SEQ ID NO: 18 - protein, 211 aa, Bacteriophage phi eiAU
  misc_feature (163)..(163): Xaa can be any naturally occurring amino acid
MHPSSRFTGGIKTVERLITIRLYGKLGAAFGRVHRRAVSTTTEAVKALCVTLDGFEEYLLHAKKNGMTFAVFRGRKNIGAEELHDGIGNDEIRIAPVIEGSKKGGLFQTILGAVLVVAGVGLTIFSGGALASFGAQMAWAGAAVMAGGLYQMLSPQPRGLQSXEDPDNRPSYAFGGPVNTTAMGNPIGVLWGTREIGGAIISAGIVAEDVA

SEQ ID NO: 19 - protein, 239 aa, Bacteriophage phi eiAU
MNKIILGEIKKHAAESGYNECCGLVVQNGRALRYIRVTNTHEMPTEHFRISAADFAAAADEGDIVRVIHSHPGDGATAEPSDADKAACNASGIIWGVYAPDCDEYREISPQDPPLIGRPFVLGADDCYGLVMAWHKRQGIDLLDFRVNYPWWERGENLYMDNWAAAGFVEADPAPGCVVIMQVRADVPNHAGVLTECGLLHHLYGRASEEIPYGGYYVDRTVLCIRHRDLPEELKPWRD

SEQ ID NO: 20 - protein, 256 aa, Bacteriophage phi eiAU
MTSYIDQSAKLDPSGRIVLVEVDASEFGAGVHRMHYAPFPHSAAEIEAAAGDEAKLGPKPIYFGGLMFDFWPFSVSGLSLSTEQAATPTITVSNLAGYLSRLCLDYRDLINAKVRVIYTYAEYLDARNFPDGNPNADPDACSYQTFWVDTKSAEDDESITWTLSSPADLQGLKIPTRQITSLCTWAMRGQYRSGDGCTYNGNAYFDAKGNPVSDPALDRCGGCYSDCVKRFGADMADPKAAALDFGGFLAAQLINR

SEQ ID NO: 21 - protein, 93 aa, Bacteriophage phi eiAU
YKKSVRSAKMGDGYEQVAENGINSVADTIALRCAGDNARMREVRAFLLRHVVKAFIFTPPGEEKGLYRVDAESVAFNLTGHTAEVTFTLNRAY

SEQ ID NO: 22 - protein, 114 aa, Bacteriophage phi eiAU
MTVETFTELCELTAPITYKKSVRSAKMGDGYEQVAENGINSVADTIALRCAGDNARMREVRAFLLRHVVKAFIFTPPGEEKGLYRVDAESVAFNLTGHTAEVTFTLNRAYGVFA

SEQ ID NO: 23 - protein, 900 aa, Bacteriophage phi eiAU
MQITEHACALIKRVQYRQHHGRADHDPRDRKHSVCAPTSAAYRKASTELDKLTVAAEKAERANDKLGDAAKKAGSGVAGAGAAAGSAATALEKNSAATERAAKAQQRQIELADKFGMSQKQLTATMRGVPAQITDIVTSLQGGQRPLTVLIQQGGQLRDMFGGIGNALRALASTIGPVGLSIAAVGATLATIGAGVTNADRQISSLNKTLNMTSHFSGLTANEILKLGESAERSGGSFRGTVSAVQKLAAAGVSANADFSALGKSVQAFAKASGQSLDDVIGQVAKLSTDPVGGLRALQTQYKAVTEEQIIRVQKLIDEGQQTRAIAEANRIASASFTDLAANVTGQLGMVELAMMSIRNAAKNMWDAILDIGRPESVGVQLAAAEKVYTAYKKRWELEKDSKVVTEAGKAALYDQMETARRQVETLRQQTQAEDKKAAAIKASALEQQKQNVLNATAASEAEKFATNTQKQNREIDTQKRLLDANLISLAEYNRRVEEIRKKYEEKPVRAKAVKVDAGVRVDEQSAAQLRALEAQIALMKQRDTYDRNASQQRRALLLFEAEHSVLVEASQKRQLTLAEKQIMASYEQIRASKVQLADAGDQLLVLQRQAEAHDNVSKAVAETDAQMQALAATYGMSTKEAKRFNDEAVTRATLAAQGATTADIEKALEAKRKLWAEQDAADKNWQAGAIKGLKDWAEASMNYADIAGQAVESAMNRGVKAVSDFVTSGKMDFKSFTADVLKMIADIITQLLVMQGIKSAANALGLGGLFANAKGGVYSGGDLSRYSGQVVNQPTMFNFDAVPKFAKGAGLMGEAGPEAIMPLKRTADGRLGISAEGGTGSSIINNISVTVSDGGAMGRATSTGGALGASIAKQMKDTVTAEVTRMLQPGGLLYKSRMA

SEQ ID NO: 24 - protein, 82 aa, Bacteriophage phi eiAU
QELYGVSPDQLIKTVEVWPDVWPVVSIFTKMAGQWRVGPCGAYALDYGVLRWMFDIHGITNQRQALDDIRVLEEVAKEEMKK

SEQ ID NO: 25 - protein, 54 aa, Bacteriophage phi eiAU
MAGQWRVGPCGAYALDYGVLRWMFDIHGITNQRQALDDIRVLEEVAKEEMKKAG

SEQ ID NO: 26 - protein, 142 aa, Bacteriophage phi eiAU
MHCNGNRAILRPPHGGYLLRGLRTMSKSPFKLNPAPTFPATVMVPNAGQDKPVPLDVVFRHYPVDEYQRNMADTYEALQDPDKDAYDVMAESLLYLLADWRVDGGDPLNKENALLLVKNFPRAYGEITKEYTTTLQCLREKN

SEQ ID NO: 27 - protein, 200 aa, Bacteriophage phi eiAU
MGYQLPNGSSVQMGATLSDPIKVIGATNAAECVFTYDESSSVAGAAVKKGDTVMLTKSPWTQALNLCGIVKAVDTAQKTITMLKLDTTDTTYYPASAFSPSVPGEMVKISGFVDFPYITNVATSGGDQQTVSFQPLQSKQAINLNTFKNPIVNTYTLTHDIEDPIRPVLEKADQTQAFAAIKFINPAAAGGKGEIRFVCR

SEQ ID NO: 28 - protein, 142 aa, Bacteriophage phi eiAU
MSVSRIRALLEGHLSAVVAGLKYPLGDILVAWENTPTDRPSLTNVMLVPNLMPAESDSISLQQTDVIYQGIFQITAMIPAGHGTRAPEKLADDIAAAFPATLMLRDASGFAVGVSGPASVFNGLATDTGYNIPISVTYRALT

SEQ ID NO: 29 - protein, 97 aa, Bacteriophage phi eiAU
MSVSRIRALLEGHLSAVVAGLKYPLGDILVAWENTPTDRPSLTNVMLVPNLMPAESDSISLQQTDVIYQGIFQITAMIPAGHGTRAPEKLADDIAAA

SEQ ID NO: 30 - protein, 210 aa, Bacteriophage phi eiAU
VTVGAFSESVRLFADKTNQRMDQVVRAFGMKILGRLITLSPVGDPSRWKVNAELSKSKARASRINAMRRKDPRRVTKTGRLKRGQKVHAGVRREFKTRNGKTVAFIQRREVGRGYTGGRFRGNWQVSFNAPIDTAIDRIDKSGGATLAAGDAVLAGLNLDQVHSVWFCNNVPYARRLEFGWSNQAPNGIVRITAAEARRYIAQAIGESKQ

SEQ ID NO: 31 - protein, 190 aa, Bacteriophage phi eiAU
MDQVVRAFGMKILGRLITLSPVGDPSRWKVNAELSKSKARASRINAMRRKDPRRVTKTGRLKRGQKVHAGVRREFKTRNGKTVAFIQRREVGRGYTGGRFRGNWQVSFNAPIDTAIDRIDKSGGATLAAGDAVLAGLNLDQVHSVWFCNNVPYARRLEFGWSNQAPNGIVRITAAEARRYIAQAIGESKQ

SEQ ID NO: 32 - protein, 118 aa, Bacteriophage phi eiAU
MALNYRKLQKTADRLLSQNGMAATVTRPSWVERVGPDEIIHPAETFTVTGVLTQYKPMEVDGTRIMAGDMRFAASGAGAEVKTGDLVTIRGKQYRVITPNPAAPNGLTVIAYNLKLRG

SEQ ID NO: 33 - protein, 166 aa, Bacteriophage phi eiAU
MLGKPEKLVAFAAERGMTITTADAAIALTKATDFINSKKWSGKKADKYQADAWPRIGIAWGDCALLDATETPIDVPEGVDPRTVTGTPQDVFTAVYRLALLCADGFDLMPSISGAQEISVSAANAVSVTYDKDTIGMRADIPWLDGLIGSWTESDGMAFGFSVSRG

SEQ ID NO: 34 - protein, 209 aa, Bacteriophage phi eiAU
MKFPTLADFPLAASLFGDAAGNIKTWAMSGTQWAQFIAYQAVPSAEKVFAIGNIEVLQDGLGRRFLISDAVGTALADVIASSTSTKLGPDAIIGLVPGAVAITTTSLDMLAEQKGGNENIERWWQGEFDFNVAVKGYRVKSALRAEIEGLRSAKLADVSSYKNWELDQGAVDNAPVKNSGGAQKVPVKNLKETAGVLMKLTATTAGAVA

SEQ ID NO: 35 - protein, 144 aa, Bacteriophage phi eiAU
MSLPVFQEKLIGTTIQLVADNLNVWNASSGGAIVMGSGTVLKDVIEKVTVGIIDGLVSDRNAYAPVGTAADAKVLARMLTNSINLSAKVGPVAITSGMMAKIQTDVNQTAGEVSALATEAIIQHYIKGAVGAVGGALCSNAASQ

SEQ ID NO: 36 - protein, 222 aa, Bacteriophage phi eiAU
MLKFKIDSAAFDALDDAVKGLYNKSGDDYVLAVEGLEDVSGLKSQVAALLNEKKTETEKRRAAEEAEKQAREEAARKAGDVDALDKSWQEKLAKVQAEAGGRTELLSKKVQDLTIGATARDLASRVFGKNAGLMLPHVAPRLSLEEVDGDFKVRVMKDGKPSAMSLDDLEKEFRTNADYAAVVVASGAGGTPKGGFQPAGGGAMPQSTLAQRATEIASGIGE

SEQ ID NO: 37 - protein, 375 aa, Bacteriophage phi eiAU
MMKASDKLADLLIRRHIFVQRFSNGQAAKVLRAIKRLAPRVAEVLAAALASEKVRGAVITPAQLRRALRKVDSTISEALRDDFAELATSMEEFADTEASFYADALTTAIRPALIPGAVVPIAAITGAQVAAAAFSAPFQGNTLLSWPDDLAAWAKRLITNQVRAGYLMGKPTMEIVAGVKATWQGKFSSGVSSVVKSAVNHYSATARELMVSANADVVKCRRWLSTLDTHTSPMCQLRDRLFYPLKVKADTEGSADRELKKHIAGSQYGAGPGKLHYCCRSTETWVIRGLDDWPDSTRPALKTDPATGRYMSESVSEGTTYFEWVQRQPRHVLEEIYGIERADQILRGLKVPKMFNDSGELYTIAQLKNKGLWRD

SEQ ID NO: 38 - protein, 486 aa, Bacteriophage phi eiAU
MAGVDTKHPDYARYAPEWARIDDCVAGERAVKAQKTKYLPHPGFDPSQDPMASKRYDSYLARAPFLNATGRTLQALLGVAFAKPVEVSLSGALDVLRENADGRGLPIAQVLRGALSAALKGGRFGFLVDFSRPAKYDAEGNPVPMTAEEAAGQRVLIDLYSAREVINWREENGRTTLVVTQRTVEVMPDDVDDFAMHSVTEYVELRLVEGVAHCRRWIHNTGATIGAYPSGFTKTDLVPLRDRDGSPLEALPWAWGGAFDNNASVDPAPLADLAGLNIKHFAAEADLAELAHVVGQPTLVVSGLTQTWVDKNLQNGIALGATRGLPLPQDSAASLLQAEDRNVCLTLCERREKQMAMIGAALIERGSAPKTATEADFDARTDNSALALAAGNVEAAFNKALEIAGRFVVGEGSVMLDRTYTALNIDPQAITALMAGVQTGVITLESFVRYLMRQGIEDDSRSVEDIMEALRVQNEPPTGGVNDEGQ

SEQ ID NO: 39 - protein, 460 aa, Bacteriophage phi eiAU
MDDFDRELLARIAKAERQVMRLGVPAPVKKERKSRTWRIKTLPHQRGLINDTTTKILGLCSGFGGGKTWSAARKAVQLAILNPGCDGIITEPTIPLLVKIMYPELEKALNEAGIKWKFNKQDKIYHCRIAGQMTRIICDSMENYTRLIGVNAAWCVCDEFDTTKPDIAMEAYRKLLGRLRTGNVRQMVIVSTPEGFRAMYQIFISEADDQKRLIKARTTDNHYLPQDYIDTLRAQYPPELIEAYLNGEFVNLTGGAVYRNFSRTLNNCDTVAEDDDTLMIGMDFNVGQMAGAVYVQRIADGVEEMHLVDEFCGLLDTDAMIDAIKERYPDHHARGLIEIFPDSSGKNRKTTNANTSDIAMLEDAGFTVSYNSVNPAVRDRVNDVNGMILNGKGQRRLKVNVARCPKATEALEQQIWDPKTGAPDKTSGVDHMADAIGYPIAFCHPIVRPAANDSIVVNFY

SEQ ID NO: 40 - protein, 165 aa, Bacteriophage phi eiAU
MALKAKLKPKVNGVRMPTKRRHGEMPEGYVYGRPTNYRPEYAEKMVQYFENATAWQLNYTDKGNAQVIPRDNQPSFVKFARLIGVTRWNLMLWARANPDFAEAYAICKELQQEFISQAAGVGLMPSAWAIFQMRANHGITDQQPDTVSDEDDSDVNVVAEADGNA

SEQ ID NO: 41 - protein, 87 aa, Bacteriophage phi eiAU
MVATGKSQTMNSRHLTGHAVDCAPLVAGAIPWNDRAPFKSVSDAMFAAAKEQGVAIRWGGDWNQNGRSDDERFYDGPHFELRRDVYP

SEQ ID NO: 42 - protein, 136 aa, Bacteriophage phi eiAU
MFKLSSRSLSRLDGVHPDLVRVVKRAIELTPVDFTVIEGRRSVERQREMVATGKSQTMNSRHLTGHAVDCAPLVAGAIPWNDRAPFKSVSDAMFAAAKEQGVAIRWGGDWNQNGRSDDERFYDGPHFELRRDVYPG

SEQ ID NO: 43 - protein, 91 aa, Bacteriophage phi eiAU
VKKPVKIEAMRFTYPPSAGFLEWCGGAVKNIRKQRHPGAVAFCDVVTLEDGPDKRARHVATEGDYIIKGVKGEFYPCKPDIFHITYDPVEE

SEQ ID NO: 44 - protein, 91 aa, Bacteriophage phi eiAU; identical to SEQ ID NO: 43

SEQ ID NO: 45 - protein, 161 aa, Bacteriophage phi eiAU
MNKTIIALLSGLALAGGLTATGYWLYQRGDTNGYERYRAEQNQRDLQALAKRKAEDDRRHAAKAEDEARALAERNQAVADADAARRTADGLRAEIAAIRRTILQYSDSQPAGSSTGKTAVLLTDVLEKSVRRNEELAAFADRSWEAANLCELSYDKQQEMR

SEQ ID NO: 46 - protein, 469 aa, Bacteriophage phi eiAU
MRMLGVPDYLLPSPHPISRAQLAEALPLIDDEELQRLHQAENDAAADEDLYVCTDEDLEDEPSRPQFTQHDPIIEGLLNFRSTWYAAGGSNIGKSFHILGTMAAVAAGIQFAGKAVIPAHCFYFDAEAPEESKRRKKALQIKYQSDLSRLHIIDTAGAGIDITTPAGRKKCVRLINDLAGEEPVGIITFDSLNATTALAAEPFDENNATDMGKVVACLKDIARETGGSPGVIHHPAKSNNNGNRTARGSGALHAAVDAAFFLEQPDPDKEHQLNFYHEKARFGMRQSPRGFILQSCKIPVDENQSELVGQYQSTAAAPDFSKELTGFEPAPFKTTPPDETLYLVPVALAPFDAGTVTPARAMANEIKEKNGKASSALYKLIEALQTLDEAPEGISQALAGSVYKKVHGDRKKFQEGWREAQEAGVVIPAANDDGEITGWLFKDWDCAPQQLSDSEKPPQPSATNSDLED

SEQ ID NO: 47 - protein, 772 aa, Bacteriophage phi eiAU
MHNAKLIRLQFAIGTSATDARPKNTTMTWRKFVDYMTDPDEIENRPTFTGSETEDEYRAKKSRQKYICASVLPGAKRGDDGVDCRSICWLDLDAVTPARLFLVRRALSRLNLRYLEYTTPGDRHPLKGEDTRSVRFAIPTDRPMAADEIWQVNTTLAHMLDVWDCADATAYQRARLMFVPHRNAAFRTGAGRTLAVDDVLAMAWEAPAEKSDRPTLSEDDLAKADENGRAIMEWCEEMGLELMPSRRGYIVECPNSANHSTDTDGTSSTAILLPNAKHPEVHFHCQHANCSGHGNINRHQHLAMRMLGVPDYLLPSPHPISRAQLAEALPLIDDEELQRLHQAENDAAADEDLYVCTDEDLEDEPSRPQFTQHDPIIEGLLNFRSTWYAAGGSNIGKSFHILGTMAAVAAGIQFAGKAVIPAHCFYFDAEAPEESKRRKKALQIKYQSDLSRLHIIDTAGAGIDITTPAGRKKCVRLINDLAGEEPVGIITFDSLNATTALAAEPFDENNATDMGKVVACLKDIARETGGSPGVIHHPAKSNNNGNRTARGSGALHAAVDAAFFLEQPDPDKEHQLNFYHEKARFGMRQSPRGFILQSCKIPVDENQSELVGQYQSTAAAPDFSKELTGFEPAPFKTTPPDETLYLVPVALAPFDAGTVTPARAMANEIKEKNGKASSALYKLIEALQTLDEAPEGISQALAGSVYKKVHGDRKKFQEGWREAQEAGVVIPAANDDGEITGWLFKDWDCAPQQLSDSEKPPQPSATNSDLED

SEQ ID NO: 48 - protein, 77 aa, Bacteriophage phi eiAU
RGRKKQSGPDLGAMRKEVEELAQVFASADDDEALEEFKKLLEDFGERTVKKISDDDLPGFHEELKKLADEFFEFEEE

SEQ ID NO: 49 - protein, 147 aa, Bacteriophage phi eiAU
MFEKLLALFERLVIAQEAIAAAGKKYYTEAEADQKMADHIEKKEASEKPKRGKKAAAAEPEDDPVDDKPKRGRKKQSGPDLGAMRKEVEELAQVFASADDDEALEEFKKLLEDFGERTVKKISDDDLPGFHEELKKLADEFFEFEEE

SEQ ID NO: 50 - protein, 214 aa, Bacteriophage phi eiAU
VAVLKAKRKNKDRSGSNEEHALLSPSSAKKWLGCPAALTAEIGIPNPSNPAAEAGTAMHAVAEIMANNLIRDGESKAASEFVGGYPLHTPTKKSKGPKFTDEMAKMVQGYIDTCVAPLVDAGAEVYIESRVDLSRPLGAPNTFGTADLVAVTELTDGSNMLIVGDLKTGRHPVDAKENRQMMIYALGLLNKYRFSHDITKVRLMIYQPFCGGVS

SEQ ID NO: 51 - protein, 157 aa, Bacteriophage phi eiAU
MHAVAEIMANNLIRDGESKAASEFVGGYPLHTPTKKSKGPKFTDEMAKMVQGYIDTCVAPLVDAGAEVYIESRVDLSRPLGAPNTFGTADLVAVTELTDGSNMLIVGDLKTGRHPVDAKENRQMMIYALGLLNKYRFSHDITKVRLMIYQPFCGGVS

SEQ ID NO: 52 - protein, 182 aa, Bacteriophage phi eiAU
LACHAAGKAALKPGDFRPSADACQWCRFREKCNAARKFNEQIAADDLRDESGDEMTPEELAEAYAKLPALRQHIKNIESATYKALLAGTKLPGLKLVAGKDGNRTWSDEALVQLRLEQGGVTPDAMYTQKLLTPTQAEKALPAGAFEWVEELITRKPGEPSIASADDKRPEYVPVKDDDLVD

SEQ ID NO: 53 - protein, 128 aa, Bacteriophage phi eiAU
MTPEELAEAYAKLPALRQHIKNIESATYKALLAGTKLPGLKLVAGKDGNRTWSDEALVQLRLEQGGVTPDAMYTQKLLTPTQAEKALPAGAFEWVEELITRKPGEPSIASADDKRPEYVPVKDDDLVD

SEQ ID NO: 54 - protein, 245 aa, Bacteriophage phi eiAU
MAKVNLKNVRLCFLHAFERAEPKNKGEKAAYKVCILLDKDDQQVEKLEDTALEVLTAKWGKREVAERWMSRNYAQDSSKECAVNDGDLREEVTPEFENAIYINARSPKQPKIQTSLGEDQTEPGITVDGDPIEGKEIYAGCYANVSIELWAQDNEHGKGLRAAILGLRFRADGEAFGGGGSTATDDDLSDDDDEPRSVSRRRSRDDEDDAPRGKSRNRRDRDEDEDDEPRERRRSVSRRRSRDDD

SEQ ID NO: 55 - protein, 735 aa, Bacteriophage phi eiAU
MPQLLFLDFETFSEADLKKVGAYAYAEHDSTEILLASYAFDDGPAKVWDATCASGESDIDLDNNSAPDDLLRGLRRAKRGRVKLVMHNGLMFDRLIIRECLGLDIPPEHIHDTMVQAFRHALPGSLDKLCEVLNVDADLAKDKAGKALIKRFCKPTPKNYKIRRYDRNTHPDEWKQFKHYARNDITAMREIYYKMPSWGEIDKENEILALDQRINDRGFYVDTDLAKAATAAVAAARAELQEAAQATYGGGLTGADFLPLLRDLAPAHHIPNAQKSTLGDLLDDADLPDEARQVIEMRLGAASTASTKYAPLLNGMSADGRRRGCLQYGGAKRTLRWAGKGFQPQNLARGYFKEKPLARGIEALKRGTAEYAFDVMKLAASTVRGCIIPAPGKKLVVADYSNVEGRGLAWLAGEDSALDTFRAGLDIYKVTAGKMFGISPDDVDGYRRQIGKACELGLGYGGGVAAFLTFSKNLGLDLEEMAVTMAGTFPDYHWRAALRAYEFMKLQEVKRKPLPGKKDDRTTVVLSKKAWLTCDCIKRMWRESHPRTVQFWYDLEEACLMAIDNPGASYWAGAKVRQDGKRAIRIERTLTRSGKPGNWLKIELPSGRILSYPGIGVSMEKTNEDDPGEKARPRIKYRGENQLTRQWGWQHTYGGKLAENVTQALCRDILAWCMLPVDNAGYEIILSVHDELITETPDTAEYNVAELERLMCDLPAWAKGFPLKAEGWEGYRYKK

SEQ ID NO: 56 - protein, 92 aa, Bacteriophage phi eiAU
MTPEGKVQAHLQRRFKAIGGLVRKISYEGRRGCPDLFIVLPGGVVVMVEVKKPGGTPEPHQVREIERLRQRGVPVYVIDSIEGADKLVAFYS

SEQ ID NO: 57 - protein, 105 aa, Bacteriophage phi eiAU
VSSASWVLLAGVMGAIVGLVVHTEIRTFRQRACFLLGGVVTAFYLSEPVGHYLALTDERSIATIGFLIGVFGMSLLQRVKETLNSLDIGAIAGARWKDLIGAFKR

SEQ ID NO: 58 - protein, 127 aa, Bacteriophage phi eiAU
MLPWPCIHITGPFLMDLWQKVSSASWVLLAGVMGAIVGLVVHTEIRTFRQRACFLLGGVVTAFYLSEPVGHYLALTDERSIATIGFLIGVFGMSLLQRVKETLNSLDIGAIAGARWKDLIGAFKRGQ

SEQ ID NO: 59 - protein, 155 aa, Bacteriophage phi eiDWF; identical to SEQ ID NO: 4

SEQ ID NO: 60 - protein, 174 aa, Bacteriophage phi eiDWF; identical to SEQ ID NO: 5

SEQ ID NO: 61 - protein, 449 aa, Bacteriophage phi eiDWF
  misc_feature (358)..(358): Xaa can be any naturally occurring amino acid
VWATMGSGKTGATMWALDAMFSTGILDESDRVLILAPLRVASGTWPEEQRKWKFPALRVIDATGNAEHRIEALATSANVVCLNYDVLEWLVEYYGNDWPFTVVVADESTRLKSYRSRGGSKRARALAKVAHKKIRRFINLTGTPAPNGLKDVWGQMWFLDAGERLGTSYQSFSDRWFVSKQVGSSPLARQISPRTGAETEIHQKCADLSITIDAAEYFGCDKPVVVPIVVELPKKARKIYDDMENALFAELESGEIEASNAAAKTAKCLQIAGGACYITTDDGEASKEWTEIHKAKLDALESIIEELNGSPLLVAYQYKHDLVRLLKRFPQGRAMRKGLKGNNDMADWNAGKVPIMFXHPASAGHGLNLQDGGCHLAFFNDTWNYEQYAQIVERIGPVRQHQAGHPRTVYIYIIQARGTLDEVVALRRDDKAEVQDLLMDYMKRKKRSK

SEQ ID NO: 62 - protein, 483 aa, Bacteriophage phi eiDWF
  misc_feature (392)..(392): Xaa can be any naturally occurring amino acid
MNISGPRGSNMAQFKRRPYQKAITGHIIAHARCNVWATMGSGKTGATMWALDAMFSTGILDESDRVLILAPLRVASGTWPEEQRKWKFPALRVIDATGNAEHRIEALATSANVVCLNYDVLEWLVEYYGNDWPFTVVVADESTRLKSYRSRGGSKRARALAKVAHKKIRRFINLTGTPAPNGLKDVWGQMWFLDAGERLGTSYQSFSDRWFVSKQVGSSPLARQISPRTGAETEIHQKCADLSITIDAAEYFGCDKPVVVPIVVELPKKARKIYDDMENALFAELESGEIEASNAAAKTAKCLQIAGGACYITTDDGEASKEWTEIHKAKLDALESIIEELNGSPLLVAYQYKHDLVRLLKRFPQGRAMRKGLKGNNDMADWNAGKVPIMFXHPASAGHGLNLQDGGCHLAFFNDTWNYEQYAQIVERIGPVRQHQAGHPRTVYIYIIQARGTLDEVVALRRDDKAEVQDLLMDYMKRKKRSK

SEQ ID NO: 63 - protein, 412 aa, Bacteriophage phi eiDWF
MTAPCGPDPALASLSARQAKVQGLLTSGTCGRPSSTSLASADLALSLVSRFQARTASAGSTLYKLTWKTRDTPAQRQIYALRASVRRTSDSDCTGWVTPTTRDWKDTGTDIKPRADGSQRFDQLPRQANLCGWPTPTANNGTGAGTSGRLGGLNLQTASLLVGPIRRTATGEILTGSAAGMECAGQLNPAHSRWLMGLPPEWDDCAPTVTPSARRSQKSSSGRISTRRKIKMSGYHDSKTAPEDKDCWRTPPEVFRYAVRTWGAFEIDAAAADHNHLVADYWTLADNALVQDWSGKRVWCNPPYSDIGPWVEKAATAEFCVMLVPADTSVKWFATAGELGASVIFITRGRLRFIHNATGKPGPSNKMGSCFLVFGGSRPGRVDFVTRAGVYQIGARRKVTVKRRVRAPHNAT

SEQ ID NO: 64 - protein, 440 aa, Bacteriophage phi eiDWF
MNSPNLPSATSSPESEYGHSPCAAQDGRMTAPCGPDPALASLSARQAKVQGLLTSGTCGRPSSTSLASADLALSLVSRFQARTASAGSTLYKLTWKTRDTPAQRQIYALRASVRRTSDSDCTGWVTPTTRDWKDTGTDIKPRADGSQRFDQLPRQANLCGWPTPTANNGTGAGTSGRLGGLNLQTASLLVGPIRRTATGEILTGSAAGMECAGQLNPAHSRWLMGLPPEWDDCAPTVTPSARRSQKSSSGRISTRRKIKMSGYHDSKTAPEDKDCWRTPPEVFRYAVRTWGAFEIDAAAADHNHLVADYWTLADNALVQDWSGKRVWCNPPYSDIGPWVEKAATAEFCVMLVPADTSVKWFATAGELGASVIFITRGRLRFIHNATGKPGPSNKMGSCFLVFGGSRPGRVDFVTRAGVYQIGARRKVTVKRRVRAPHNAT

SEQ ID NO: 65 - protein, 204 aa, Bacteriophage phi eiDWF
VAKAGAAGLGGAVGGLIGSWFGNGFGGGWGNRGGVAGEAAIATTAVLDGINSVASAVNAGTLQTLQGQNGTNMTIANGLSAAQYANFQGQAGIQSALCQGFAGVNATVDRNGADTRFAVASGFAGVSADMARCCCETQKTIAAEGAATRQLIQQNFITDLQTQLCDQKAQNAKLSGEIFLQNSQAAQTNQIINTILAHLGTKSA

SEQ ID NO: 66 - protein, 132 aa, Bacteriophage phi eiDWF
MTIANGLSAAQYANFQGQAGIQSALCQGFAGVNATVDRNGADTRFAVASGFAGVSADMARCCCETQKTIAAEGAATRQLIQQNFITDLQTQLCDQKAQNAKLSGEIFLQNSQAAQTNQIINTILAHLGTKSA

SEQ ID NO: 67 - protein, 117 aa, Bacteriophage phi eiDWF; identical to SEQ ID NO: 12

SEQ ID NO: 68 - protein, 335 aa, Bacteriophage phi eiDWF; identical to SEQ ID NO: 14

SEQ ID NO: 69 - protein, 1203 aa, Bacteriophage phi eiDWF
  misc_feature (1111)..(1111): Xaa can be any naturally occurring amino acid
MQRQLFYIKGAKGGGGDAHTPVEQPDSIRSQAKARLLIALGEGEMALGLDDTKIFLDGTPLGNPDGSRNFDGVRWEVRPGVQQQDPISGFPAVENETGFGTEIKQASPWVHALTRTEIDAVVVRVGVPALMYQEDDGDVVGTSVSFRIDLAVGGGTFSTQGKFAISGKTTTLYERSIRVNLPRSSSGWRIRVVRETPDSDSARLANTLKIQAITEVIDARFRYPHTALLFIEFNAKSFQNIPKISCLAKGRIIRVPSNYDPDTRTYSGNWDGSFKWAYTNNPAWVWYDVLTQPRFGLGKRVTAAMLDKWELYRIAQRCDQMVPDGAGGVEPRFEFNCYLQAQADAWTVIRDIAAGFNGLTYWGNNMFNVVSDMPVKAPSQIVTRASIIGKPTYSSGSRKTRFSSALVNYSDAQNHYADTPTAVMFQELVAQLGFEQTQLTAIGCTRESEAQRRASWAVLTNSVDRLVKLRVGLEGFAFLPGTVFALADERIGGRVMGGRVAGYDEKTKQVMLDRTTDGKPGDDLLIRTTGGAVESRKIASVGDSVVTIAEPFTAAPAVNAVWVVDSGELALQKFRVLTLDFDDENNTFEISAAEYNDSKYDAVDDGARLDKPPVSLLPTGIVNAPTAVAITSYEQVRQNQRVTTMRATWEPSRMADGKVQPDIVAYEAQWRRGANDWVNVPASSVNGFEVQGVFAGDYLVRVRAVTSFGASSVWASSVLTHIDGRQGEVPAPVSLRASSDVVFGIDVAWAFPKDAEDTEYTEIQYAPTNTEEAFTTLSLSPYPSKSFAHSGLKANAVFWYRARLVDRLGNKSEWGASVQGRASIDTDSIMDALGDQVMSSEGGKALETSINAAIDAIEQNAIANDGDIQRKSKKLGELSAEIVRIDNVVVNEVGALAESLTAVKASVAENEAAVATKMTAKFDYDGNGYAVWDTNAGITYNGEYYSAGMSISAEVKEGEVSTQVAMLADRFAVMAKVGDKPELMFGVVGDQAYLRDAFIRDASIGSAKIAGVLQSDDYTPGGAGWTINKSGAVEFNNATIRGTVYAENGDFKGTVHANRIVGDVVQYSNFTFSSKDVSVGNGATRVLFKVPAEDFEQTIISNGYVKFFAGXGGMTRISCYVESSGVRKVLTELWSNGXTAEYKFNLSGLTLPPGANGTWIRIEFTKTWPNTIRPEKPHTLLTYDGAQLLMGRARRGSAEILEG

SEQ ID NO: 70 - protein, 1225 aa, Bacteriophage phi eiDWF
  misc_feature (1133)..(1133): Xaa can be any naturally occurring amino acid
MGDERDRRCYNQCRNCCRGRSIMQRQLFYIKGAKGGGGDAHTPVEQPDSIRSQAKARLLIALGEGEMALGLDDTKIFLDGTPLGNPDGSRNFDGVRWEVRPGVQQQDPISGFPAVENETGFGTEIKQASPWVHALTRTEIDAVVVRVGVPALMYQEDDGDVVGTSVSFRIDLAVGGGTFSTQGKFAISGKTTTLYERSIRVNLPRSSSGWRIRVVRETPDSDSARLANTLKIQAITEVIDARFRYPHTALLFIEFNAKSFQNIPKISCLAKGRIIRVPSNYDPDTRTYSGNWDGSFKWAYTNNPAWVWYDVLTQPRFGLGKRVTAAMLDKWELYRIAQRCDQMVPDGAGGVEPRFEFNCYLQAQADAWTVIRDIAAGFNGLTYWGNNMFNVVSDMPVKAPSQIVTRASIIGKPTYSSGSRKTRFSSALVNYSDAQNHYADTPTAVMFQELVAQLGFEQTQLTAIGCTRESEAQRRASWAVLTNSVDRLVKLRVGLEGFAFLPGTVFALADERIGGRVMGGRVAGYDEKTKQVMLDRTTDGKPGDDLLIRTTGGAVESRKIASVGDSVVTIAEPFTAAPAVNAVWVVDSGELALQKFRVLTLDFDDENNTFEISAAEYNDSKYDAVDDGARLDKPPVSLLPTGIVNAPTAVAITSYEQVRQNQRVTTMRATWEPSRMADGKVQPDIVAYEAQWRRGANDWVNVPASSVNGFEVQGVFAGDYLVRVRAVTSFGASSVWASSVLTHIDGRQGEVPAPVSLRASSDVVFGIDVAWAFPKDAEDTEYTEIQYAPTNTEEAFTTLSLSPYPSKSFAHSGLKANAVFWYRARLVDRLGNKSEWGASVQGRASIDTDSIMDALGDQVMSSEGGKALETSINAAIDAIEQNAIANDGDIQRKSKKLGELSAEIVRIDNVVVNEVGALAESLTAVKASVAENEAAVATKMTAKFDYDGNGYAVWDTNAGITYNGEYYSAGMSISAEVKEGEVSTQVAMLADRFAVMAKVGDKPELMFGVVGDQAYLRDAFIRDASIGSAKIAGVLQSDDYTPGGAGWTINKSGAVEFNNATIRGTVYAENGDFKGTVHANRIVGDVVQYSNFTFSSKDVSVGNGATRVLFKVPAEDFEQTIISNGYVKFFAGXGGMTRISCYVESSGVRKVLTELWSNGXTAEYKFNLSGLTLPPGANGTWIRIEFTKTWPNTIRPEKPHTLLTYDGAQLLMGRARRGSAEILEG

SEQ ID NO: 71 - protein, 174 aa, Bacteriophage phi eiDWF
  misc_feature (29)..(29): Xaa can be any naturally occurring amino acid
VSTTTEAVKALCVTLDGFEEYLLHAKKNXMTFAVFRGRKNIGAEELHDGIGNDEIRIAPVIEGSKKGGLFQTILGAVLVVAGVGLTIFSGGALASFGAQMAWAGAAVMAGGLYQMLSPQPRGLQSREDPDNRPSYAFGGPVNTTAMGNPIGVLWGTREIGGAIISAGIVAEDVA

SEQ ID NO: 72 - protein, 211 aa, Bacteriophage phi eiDWF
  misc_feature (66)..(66): Xaa can be any naturally occurring amino acid
MHPSSRFTGGIKTVERLITIRLYGKLGAAFGRVHRRAVSTTTEAVKALCVTLDGFEEYLLHAKKNXMTFAVFRGRKNIGAEELHDGIGNDEIRIAPVIEGSKKGGLFQTILGAVLVVAGVGLTIFSGGALASFGAQMAWAGAAVMAGGLYQMLSPQPRGLQSREDPDNRPSYAFGGPVNTTAMGNPIGVLWGTREIGGAIISAGIVAEDVA

SEQ ID NO: 73 - protein, 239 aa, Bacteriophage phi eiDWF; identical to SEQ ID NO: 19

SEQ ID NO: 74 - protein, 256 aa, Bacteriophage phi eiDWF; identical to SEQ ID NO: 20

SEQ ID NO: 75 - protein, 114 aa, Bacteriophage phi eiDWF; identical to SEQ ID NO: 22

SEQ ID NO: 76 - protein, 866 aa, Bacteriophage phi eiDWF
LRADVGSVSKASTELDKLTVAAEKAERANDKLGDAAKKAGSGVAGAGAAAGSAATALEKNSAATERAAKAQQRQIELADKFGMSQKQLTATMRGVPAQITDIVTSLQGGQRPLTVLIQQGGQLRDMFGGIGNALRALASTIGPVGLSIAAVGATLATIGAGVTNADRQISSLNKTLNMTSHFSGLTANEILKLGESAERSGGSFRGTVSAVQKLAAAGVSANADFSALGKSVQAFAKASGQSLDDVIGQVAKLSTDPVGGLRALQTQYKAVTEEQIIRVQKLIDEGQQTRAIAEANRIASASFTDLAANVTGQLGMVELAMMSIRNAAKNMWDAILDIGRPESVGVQLAAAEKVYTAYKKRWELEKDSKVVTEAGKAALYDQMETARRQVETLRQQTQAEDKKAAAIKASALEQQKQNVLNATAASEAEKFATNTQKQNREIDTQKRLLDANLISLAEYNRRVEEIRKKYEEKPVRAKAVKVDAGVRVDEQSAAQLRALEAQIALMKQRDTYDRNASQQRRALLLFEAEHSVLVEASQKRQLTLAEKQIMASYEQIRASKVQLADAGDQLLVLQRQAEAHDNVSKAVAETDAQMQALAATYGMSTKEAKRFNDEAVTRATLAAQGATTADIEKALEAKRKLWAEQDAADKNWQAGAIKGLKDWAEASMNYADIAGQAVESAMNRGVKAVSDFVTSGKMDFKSFTADVLKMIADIITQLLVMQGIKSAANALGLGGLFANAKGGVYSGGDLSRYSGQVVNQPTMFNFDAVPKFAKGAGLMGEAGPEAIMPLKRTADGRLGISAEGGTGSSIINNISVTVSDGGAMGRATSTGGALGASIAKQMKDTVTAEVTRMLQPGGLLYKSRMA

SEQ ID NO: 77 - protein, 784 aa, Bacteriophage phi eiDWF
MSQKQLTATMRGVPAQITDIVTSLQGGQRPLTVLIQQGGQLRDMFGGIGNALRALASTIGPVGLSIAAVGATLATIGAGVTNADRQISSLNKTLNMTSHFSGLTANEILKLGESAERSGGSFRGTVSAVQKLAAAGVSANADFSALGKSVQAFAKASGQSLDDVIGQVAKLSTDPVGGLRALQTQYKAVTEEQIIRVQKLIDEGQQTRAIAEANRIASASFTDLAANVTGQLGMVELAMMSIRNAAKNMWDAILDIGRPESVGVQLAAAEKVYTAYKKRWELEKDSKVVTEAGKAALYDQMETARRQVETLRQQTQAEDKKAAAIKASALEQQKQNVLNATAASEAEKFATNTQKQNREIDTQKRLLDANLISLAEYNRRVEEIRKKYEEKPVRAKAVKVDAGVRVDEQSAAQLRALEAQIALMKQRDTYDRNASQQRRALLLFEAEHSVLVEASQKRQLTLAEKQIMASYEQIRASKVQLADAGDQLLVLQRQAEAHDNVSKAVAETDAQMQALAATYGMSTKEAKRFNDEAVTRATLAAQGATTADIEKALEAKRKLWAEQDAADKNWQAGAIKGLKDWAEASMNYADIAGQAVESAMNRGVKAVSDFVTSGKMDFKSFTADVLKMIADIITQLLVMQGIKSAANALGLGGLFANAKGGVYSGGDLSRYSGQVVNQPTMFNFDAVPKFAKGAGLMGEAGPEAIMPLKRTADGRLGISAEGGTGSSIINNISVTVSDGGAMGRATSTGGALGASIAKQMKDTVTAEVTRMLQPGGLLYKSRMA

SEQ ID NO: 78 - protein, 108 aa, Bacteriophage phi eiDWF
VPPRKKLREAAAALYAVPPSEDIAQELYGVSPDQLIKTVEVWPDVWPVVSIFTKMAGQWRVGPCGAYALDYGVLRWMFDIHGITDQRQALDDIRVLEEVAKEEMKKAG

SEQ ID NO: 79 - protein, 54 aa, Bacteriophage phi eiDWF
MAGQWRVGPCGAYALDYGVLRWMFDIHGITDQRQALDDIRVLEEVAKEEMKKAG

SEQ ID NO: 80 - protein, 142 aa, Bacteriophage phi eiDWF
MHCNGNRAILRPPHGGFLLRGLRTMSKSPFKLNPAPTFPATVMVPIAGQDKPVPLDVVFRHYPVDEYQRNMADTYEALQDPDKDAYDVMAESLLYLLADWRVDGGDPLNKENALLLVKNFPRAYGEITKEYTTTLQCLREKN

SEQ ID NO: 81 - protein, 239 aa, Bacteriophage phi eiDWF
MGYQLPNGSSVQMGATLSDPIKVIGATNAAECVFTYDESSSVAGAAVKKGDTVMLTKSPWTQALNLCGIVKAVDTAQKTITMLKLDTTDTTYYPASAFSPSVPGEMVKISGFVDFPYITNVATSGGDQQTVSFQPLQSKQAINLNTFKNPIVNTYTLTHDIEDPIRPVLEKADQTQAFAAIKFINPAAAGGKGEIRLYAAKVSFQPIPSSEVNNVETVSVALSMQSGMRIYIKSEVDAL

SEQ ID NO: 82 - protein, 142 aa, Bacteriophage phi eiDWF; identical to SEQ ID NO: 28

SEQ ID NO: 83 - protein, 208 aa, Bacteriophage phi eiDWF
VGAFSESVRLFADKTNQRMDQVVRAFGMKILGRLITLSPVGDPSRWKVNAELSKSKARASRINAMRRKDPRRVTKTGRLKRGQKVHAGVRREFKTRNGKTVAFIQRREVGRGYTGGRFRGNWQVSFNAPIDTAIDRIDKSGGATLAAGDAVLAGLNLDQVHSVWFCNNVPYARRLEFGWSNQAPNGIVRITAAEARRYIAQAIGESKQ

SEQ ID NO: 84 - protein, 190 aa, Bacteriophage phi eiDWF; identical to SEQ ID NO: 31

SEQ ID NO: 85 - protein, 118 aa, Bacteriophage phi eiDWF
MALNYRKLQKTADRLLSQNGMAATVTRPAWVERVGVDEIIHPAETFTITGVLAQYKPMEIDGTRIMAGDVRFAASGAGAEVKTGDLVTILGKQYRVITPNPAAPNGSTVIAYNLQLRG

SEQ ID NO: 86 - protein, 119 aa, Bacteriophage phi eiDWF
MMALNYRKLQKTADRLLSQNGMAATVTRPAWVERVGVDEIIHPAETFTITGVLAQYKPMEIDGTRIMAGDVRFAASGAGAEVKTGDLVTILGKQYRVITPNPAAPNGSTVIAYNLQLRG

SEQ ID NO: 87 - protein, 166 aa, Bacteriophage phi eiDWF; identical to SEQ ID NO: 33

SEQ ID NO: 88 - protein, 366 aa, Bacteriophage phi eiDWF
MSLPVFQEKLIGTTIQLVADNLNVWNASSGGAIVMGSGTVLKDVIEKVTVGIIDGLVSDRNAYAPVGTAADAKVLARMLTNSINLSAKVGPVAITSGMMAKIQTDVNQTAGEVSALATEAIIQHYIKGAVGAVGGALCSNAASQYTQPARVNVTATGMKFPTLADFPLAASLFGDAAGNIKTWAMSGTQWAQFIAYQAVPSAEKVFAIGNIEVLQDGLGRRFLISDAVGTALADVIASSTSTKLGPDAIIGLVPGAVAITTTGLDMLAEQKGGNENIERWWQGEFDFNVAVKGYRIKSSLRTEIEGLRSAKLADVSSYKNWELDQGAVDNAPVKNTGGSQKVPVKNLKETAGVLMKLTATTAGAAV

SEQ ID NO: 89 - protein, 222 aa, Bacteriophage phi eiDWF
MLKFKIDSAAFDALDDAVKGLYNKSGDDYVLAVEGLEDVSGLKSQVAALLNEKKTEAEKRRAAEEAEKQAREEAARKAGDVEALDKSWQEKLAKVQAEASGRTELLSKKVQDLTIGATARDLASRVFGKNAGLMLPHVAPRLSLEEVDGDFKVRVMKDGKPSAMSLDDLEKEFRTNADYAAVVVASGAGGTPKGGFQPAGGGAMPQSTLAQRATEIASGIGE

SEQ ID NO: 90 - protein, 375 aa, Bacteriophage phi eiDWF; identical to SEQ ID NO: 37

SEQ ID NO: 91 - protein, 486 aa, Bacteriophage phi eiDWF
MAGVDTKHPDYARYAPEWARIDDCVAGERAVKAQKTKYLPHPAFDPSQDPMASKRYDSYLARAPFLNATGRTLQALLGVAFAKPVEVSLSGALDVLRENADGRGLPIAQVLRGALSAALKGGRFGFLVDFSRPAKYDAEGNPVPMTAEEAAGQRVLIDLYSAREVINWREENGRTTLVVTQRTVEVMPDDVDDFAMHSVTEYVELRLVEGVAHCRRWIHNTGATIGAYPSGFTKTDLVPLRDRDGSPLEELPWAWGGAFDNNASVDPAPLADLAGLNIKHFAAEADLAELAHVVGQPTLVVSGLTQTWVDKNLQNGIALGATRGLPLPQDSAASLLQAEDRNVCLTLCERREKQMAMIGAALIERGSAPKTATEADYDARTDNSALALAAGNVEAAFNKALEIAGRFVVGEGSVMLDRTYTALNIDPQAITALMAGVQTGVITLESFVRYLMRQGIEDDSRSVEDIMEALRVQNEPPTGGVNDEGQ

SEQ ID NO: 92 - protein, 460 aa, Bacteriophage phi eiDWF
MDDFDRELLARIAKAERQVMRLGVPAPVKKERKSRTWRIKTLPHQRGLINDTTTKILGLCSGFGGGKTWSAARKAVQLAILNPGCDGIITEPTIPLLVKIMYPELEKALNEAGIKWKFNKQDKIYHCRIAGQMTRIICDSMENYTRLIGVNAAWCVCDEFDTTKPDIAMEAYRKLLGRLRTGNVRQMVIVSTPEGFRAMYQIFISEADDQKRLIKARTTDNHYLPQDYIDTLRAQYPPELIEAYLNGEFVNLTGGAVYRNFSRTLNNCDTVAEDDDTLMIGMDFNVGQMAGAVYVQRIADGVEEMHLVDEFCGLLDTDAMIDAIKERYPDHHARGLIEIFPDSSGKNRKTTNANTSDIAMLEDAGFTVSYNSVNPAVRDRVNDVNGMILNGKGQRRLKVNVARCPKATEALEQQIWDPKTGAPDKTSGVDHMADAIGYPIAFRHPIVRPAANDSIVVNFY

SEQ ID NO: 93 - protein, 165 aa, Bacteriophage phi eiDWF; identical to SEQ ID NO: 40

SEQ ID NO: 94 - protein, 88 aa, Bacteriophage phi eiDWF
MVATGKSQTMNSRHLTGHAVDCAPLVAGAIPWNDRAPFKSVSDAMFAAAKEQGVAIRWGGDWNQNGRSDDERFYDGPHFELRRDVYPG

SEQ ID NO: 95 - protein, 136 aa, Bacteriophage phi eiDWF; identical to SEQ ID NO: 42

SEQ ID NO: 96 - protein, 161 aa, Bacteriophage phi eiDWF; identical to SEQ ID NO: 45

SEQ ID NO: 97 - protein, 469 aa, Bacteriophage phi eiDWF; identical to SEQ ID NO: 46

SEQ ID NO: 98 - protein, 629 aa, Bacteriophage phi eiDWF
MAADEIWQVNTTLAHMLDVWDCADATAYQRARLMFVPHRNAAFRTGAGRTLAVDDVLAMAWEAPAEKSDRPTLSEDDLAKADENGRAIMEWCEEMGLELMPSRRGYIVECPNSANHSTDTDGTSSTAILLPNAKHPEVHFHCQHANCSGHGNINRHQHLAMRMLGVPDYLLPSPHPISRAQLAEALPLIDDEELQRLHQAENDAAADEDLYVCTDEDLEDEPSRPQFTQHDPIIEGLLNFRSTWYAAGGSNIGKSFHILGTMAAVAAGIQFAGKAVIPAHCFYFDAEAPEESKRRKKALQIKYQSDLSRLHIIDTAGAGIDITTPAGRKKCVRLINDLAGEEPVGIITFDSLNATTALAAEPFDENNATDMGKVVACLKDIARETGGSPGVIHHPAKSNNNGNRTARGSGALHAAVDAAFFLEQPDPDKEHQLNFYHEKARFGMRQSPRGFILQSCKIPVDENQSELVGQYQSTAAAPDFSKELTGFEPAPFKTTPPDETLYLVPVALAPFDAGTVTPARAMANEIKEKNGKASSALYKLIEALQTLDEAPEGISQALAGSVYKKVHGDRKKFQEGWREAQEAGVVIPAANDDGEITGWLFKDWDCAPQQLSDSEKPPQPSATNSDLED

SEQ ID NO: 99 - protein, 64 aa, Bacteriophage phi eiDWF
MRKEVEELAQVFASADDDEALEEFKKLLEDFGERTVKKISDDDLPGFHEELKKLADEFFEFEEE

SEQ ID NO: 100 - protein, 419 aa, Bacteriophage phi eiDWF
VAVLKAKRKNKDRSGSNEEHALLSPSSAKKWLGCPAALTAEIGIPNPSNPAAEAGTAMHAVAEIMANNLIRDGESKAASEFVGGYPLHTPTKKSKGPKFTDEMAKMVQGYIDTCVAPLVDAGAEVYIESRVDLSRPLGAPNTFGTADLVAVTELTDGSNMLIVGDLKTGRHPVDAKENRQMMIYALGLLNKYRFSHDITKVRLMIYQPFCGGVSEWDTSAEVIETFGKFAKDRAAKALACHAAGKAALKPGDFRPSADACQWCRFREKCNAARKFNEQIAADDLRDESGDEMTPEELAEAYAKLPALRQHIKNIESATYKALLAGTKLPGLKLVAGKDGNRTWSDEALVQLRLEQGGVTPDAMYTQKLLTPTQAEKALPAGAFEWVEELITRKPGEPSIASADDKRPEYVPVKDDDLVD

SEQ ID NO: 101 - protein, 362 aa, Bacteriophage phi eiDWF
MHAVAEIMANNLIRDGESKAASEFVGGYPLHTPTKKSKGPKFTDEMAKMVQGYIDTCVAPLVDAGAEVYIESRVDLSRPLGAPNTFGTADLVAVTELTDGSNMLIVGDLKTGRHPVDAKENRQMMIYALGLLNKYRFSHDITKVRLMIYQPFCGGVSEWDTSAEVIETFGKFAKDRAAKALACHAAGKAALKPGDFRPSADACQWCRFREKCNAARKFNEQIAADDLRDESGDEMTPEELAEAYAKLPALRQHIKNIESATYKALLAGTKLPGLKLVAGKDGNRTWSDEALVQLRLEQGGVTPDAMYTQKLLTPTQAEKALPAGAFEWVEELITRKPGEPSIASADDKRPEYVPVKDDDLVD

SEQ ID NO: 102 - protein, 245 aa, Bacteriophage phi eiDWF; identical to SEQ ID NO: 54

SEQ ID NO: 103 - protein, 735 aa, Bacteriophage phi eiDWF; identical to SEQ ID NO: 55

SEQ ID NO: 104 - protein, 92 aa, Bacteriophage phi eiDWF; identical to SEQ ID NO: 56

SEQ ID NO: 105 - protein, 102 aa, Bacteriophage phi eiDWF
  misc_feature (21)..(21): Xaa can be any naturally occurring amino acid
WVLLAGVMGAIVGLVVHTEIXTFRQRACFLLGGVVTAFYLSEPVGHYLALTDERSIATIGFLIGVFGMSLLQRVKETLNSLDIGAIAAARWKDLIGAFKRGQ

SEQ ID NO: 106 - protein, 127 aa, Bacteriophage phi eiDWF
  misc_feature (46)..(46): Xaa can be any naturally occurring amino acid
MLPWPCIHITGPFLMDLWQKVSSASWVLLAGVMGAIVGLVVHTEIXTFRQRACFLLGGVVTAFYLSEPVGHYLALTDERSIATIGFLIGVFGMSLLQRVKETLNSLDIGAIAAARWKDLIGAFKRGQ

(19) United States
(12) Patent Application Publication
(10) Pub. No.: US 2010/0092431 A1
(43) Pub. Date: Apr. 15, 2010

(54) Edwardsiella Ictaluri Bacteriophage and Uses Thereof

(75) Inventors: MARK R. LILES, AUBURN, AL (US); JOHN K. WALAKIRA, Kampala (UG); ABEL A. CARRIAS, AUBURN, AL (US); JEFFERY S. TERHUNE, AUBURN, AL (US)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: AUBURN UNIVERSITY, AUBURN, AL (US)

(21) Appl. No.: 12/466,165

(22) Filed: May 14, 2009

Related U.S. Application Data
(60) Provisional application No. 61/127,786, filed on May 15, 2008.

(51) Int. Cl. A61K 35/76; C12N 7/00; C07H 21/04; C12N 1/21; C12P 21/06; C12N 15/63; C12N 9/00; C07K 14/00

(52) U.S. Cl. 424/93.6; 435/235.1; 536/23.1; 435/252.3; 435/69.1; 435/320.1; 435/183; 530/300

(57) ABSTRACT

Disclosed are isolated bacteriophage that have lytic activity for species of Edwardsiella bacteria including Edwardsiella ictaluri. The disclosed bacteriophage have been designated “ΦeiAU” and “ΦeiDWF.” Also disclosed are variant bacteriophage of ΦeiAU and ΦeiDWF bacteriophage, which variant bacteriophage have lytic activity against Edw. ictaluri. Also disclosed are isolated Edwardsiella ictaluri bacteriophage polynucleotides and polypeptides.

### CROSS-REFERENCE TO RELATED APPLICATIONS

The present application claims the benefit of priority under 35 U.S.C. §119(e) to U.S. provisional application No. 61/127,786, filed on May 15, 2008, the content of which is incorporated herein by reference in its entirety.

### BACKGROUND

The present invention relates to novel bacteriophage, polynucleotides, polypeptides, and compositions comprising the same. More specifically, isolated Edwardsiella ictaluri bacteriophage compositions are provided having lytic specificity for Edwardsiella ictaluri bacteria, which phage are useful for controlling or inhibiting the growth of Edwardsiella ictaluri bacteria. The invention also relates to methods of using Edwardsiella ictaluri bacteriophage for the removal of Edwardsiella ictaluri bacteria from environments where the bacteria may be passed to animals. The invention is also related to methods of using Edwardsiella ictaluri bacteriophage to treat or prevent diseases caused by Edwardsiella ictaluri bacteria. The present invention also relates to methods of detecting the presence of Edwardsiella ictaluri bacteria.

Bacteriophage derive their name from the Greek word “phage” which means “to eat.” Hence, “bacteriophage” literally means bacteria eaters. Many bacteriophage are lytic to the bacteria which they infect, and therefore, active bacteriophage infection produce plaques in lawns of bacteria grown on Petri dishes. Bacteriophage generally are grouped into nine phylogenetic families which including the Myoviridae (e.g., T-even bacteriophage), Styloviridae (e.g., Lambda bacteriophage groups), Podoviridae (e.g., T-7 and related bacteriophage), Microviridae (e.g., X174 group), Leviviridae (e.g., MS2), Inoviridae, Cystoviridae, Microviridae, and Siphoviridae families.

Edwardsiella ictaluri is the causative agent of enteric septicemia of catfish (ESC) and is one of the leading fish pathogens affecting farm-raised channel catfish (Ictalurus punctatus Rafinesque) in the southeastern states of the United States (Hawke et al. 1981, Hawke et al. 1998, Plumb 1999, Hawke & Khoo 2004). Economic loses due directly to ESC outbreaks are estimated between $20 and $30 million per year, affecting 78% of all aquaculture farms (Wagner et al. 2002, USDA 2003a, USDA 2003b). The disease primarily affects channel catfish but has also been experimentally reisolated from other species: walking catfish (Clarias batrachus Linnaeus), European catfish (Silurus glanis Linnaeus), Chinook salmon (Oncorhynchus tshawytscha Walbaum) and rainbow trout (Oncorhynchus mykiss Walbaum) (Inglis et al. 1993, Plumb 1999). ESC outbreaks are seasonal with occurrences during late spring and early fall when temperatures range from 18° C. to 30° C. (Tucker & Robinson 1990, Hawke et al. 1998). However, adverse environmental conditions that exist in an aquaculture system can greatly accelerate the severity of ESC causing mortalities of over 50% of cultured fish (Plumb 1999).

Control and preventive measures against ESC such as the application of antibiotics and a vaccine are available (Wise & Johnson 1998, Klesius & Shoemaker 1999, Shoemaker et al. 1999, Wise & Terhune 2001) but have not been adopted by all catfish producers. Application of medicated feed is an expensive practice and is marginally effective. Antibiotic-resistance of Edw. ictaluri to oxytetracycline and ormethoprim-sulphadimethoxine (drugs approved for use in catfish) raises concerns about the long-term efficacy of antibiotic treatment in commercial production (Johnson 1991, DePaola et al. 1995, Plumb et al. 1995). Similarly, disease outbreaks often occur within vaccinated catfish populations (Thune et al. 1994).

Biological control agents such as bacteriophages may provide an alternative mechanism to control bacterial diseases in both human and veterinary medicine (Barrow 2001, Barrow & Soothhill 1997). Phage therapy typically involves isolation of diverse bacteriophages specific to a bacterial pathogen that can be used in combination as a bacteriophage “cocktail” (Sulakvelidze et al. 2001). Because a phage can exhibit strong host specificity, express efficient systems for host cell lysis, and spread avidly within an aquatic medium, there has been an increasing interest in their use in the aquaculture industry to control fish pathogens. Studies have demonstrated that in vitro and in vivo challenges with bacteriophages may reduce mortalities in yellowtail (Seriola quinqueradiata Temminck & Schlegel), Ayu fish (Plecoglossus altivelis Temminck & Schlegel), abalone (Haliotis discus hannai Ino), loaches (Misgurnus anguillicaudatus Cantor), brook trout (Salvelinus fontinalis Mitchill) and eastern oysters (Crassostrea virginica Gmelin) (Wu et al. 1981, 1984, Li et al. 1999, Nakai et al. 1999, Tai-wu 2000, Pelon et al. 2005, Imbeault et al. 2006).

Two principal challenges in the use of bacteriophages as biological control agents are the selection for bacterial resistance to phage infection, and rapid clearance of phage by the fish reticuloendothelial system (Russell et al. 1976, Nakai & Park 2002, Levin & Bull 2004, Dabrowska et al. 2005). Bacterial resistance to phage infection may be lessened as a problem by using phage cocktails that include phages that target diverse host cell receptors. Furthermore, selection for phage-resistance may result in avirulent Edw. ictaluri phenotypes depending upon the mechanism of phage-resistance (i.e. whether the phage receptor is required for bacterial virulence). Such loss of bacterial virulence in a phage-resistant bacterial mutant has been demonstrated previously in a fish pathogen (Park et al. 2000). The problem of reticuloendothelial system clearance of phage within fish may be lessened by selecting for phage variants with reduced clearance rates, via serial passaging of phage within the animal host as has been demonstrated with long-circulating phage variants in a mouse model (Merril et al. 1996). Therefore, the ability to control an aquaculture pathogen through the use of bacteriophage therapy will depend upon several factors, including the route of pathogen infection into an animal host, having multiple phage types that infect diverse genomovars of the bacterial pathogen, the kinetics of phage infection of the bacterial host, burst size of the phage, and whether the phage can enter a lysogenic stage.

While ESC is in some respects an ideal bacterial disease for bacteriophage therapy (i.e. high-density of catfish in aquaculture ponds, fecal-oral route of infection, closed aquatic system), no phage that infects Edw. ictaluri has ever been reported. Clearly, not every phage isolated would be an attractive candidate for phage therapy of ESC. Hence, this study focused on isolating bacteriophages with Edw. ictaluri host-specificity, without evidence of lysogeny, and capable of producing clear plaques upon pathogenic strains of Edw. ictaluri.

### SUMMARY

Disclosed are isolated bacteriophage that have lytic activity for species of Edwardsiella bacteria including Edwardsiella ictaluri. The disclosed bacteriophage have been designated “ΦeiAU” and “ΦeiDWF.” Also disclosed are variants of ΦeiAU and ΦeiDWF bacteriophage, which variant bacteriophage share genotypic and phylogenetic characteristics with ΦeiAU and ΦeiDWF, including having lytic activity against Edw. ictaluri. Also disclosed are isolated Edwardsiella ictaluri bacteriophage polynucleotides, polypeptides, and compositions comprising the same.

The disclosed bacteriophage comprise a double-stranded circular DNA genome of about 40-45 kb (commonly 41-43 kb) which genome may comprise, for example, a polynucleotide sequence of one of SEQ ID NOs:1-3 or the reverse complement thereof. A variant bacteriophage may comprise a double-stranded circular DNA genome of about 40-45 kb (or about 41-43 kb) which variant genome comprises a variant polynucleotide sequence of one of SEQ ID NOs:1-3. In some embodiments, a variant bacteriophage comprises a full-length variant polynucleotide sequence of one of SEQ ID NOs:1-3 based on degeneracy of the genetic code, wherein the variant bacteriophage has lytic activity against Edw. ictaluri. In further embodiments, the variant bacteriophage has a genome comprising a polynucleotide sequence that is a full-length variant of one of SEQ ID NOs:1-3, having at least 95% sequence identity to one of SEQ ID NOs:1-3, respectively, (preferably at least 96%, 97%, 98%, or 99% sequence identity to one of SEQ ID NOs:1-3, respectively), wherein the variant bacteriophage has lytic activity against Edw. ictaluri.

The disclosed bacteriophage and variants thereof exhibit lytic activity in various species of Edwardsiella bacteria including Edwardsiella ictaluri. In some embodiments, the disclosed bacteriophage or variants thereof may be utilized in methods for killing Edw. ictaluri bacteria in which the bacteria are contacted with the disclosed bacteriophage. The methods may be utilized to control or prevent the infection or colonization of catfish (e.g., Ictaluri punctatus Rafinesque) by Edw. ictaluri, or colonization of environments in which catfish live or are raised (e.g., aquaculture ponds). The disclosed methods also may be utilized to detect the presence of Edw. ictaluri bacteria in a sample (e.g., a sample obtained from an infected catfish or a sample isolated from an environment in which catfish live or are raised). Also disclosed are methods of using Edw. ictaluri bacteriophage for removing Edw. ictaluri from environments or instruments used to raise catfish, thereby reducing the likelihood that the bacteria may be passed to the catfish. Also disclosed are methods of using Edw. ictaluri bacteriophage to treat or prevent diseases caused by Edw. ictaluri (e.g., treating or preventing enteric septicemia of catfish (ESC)). In further embodiments, in order to control or inhibit the growth of Edwardsiella ictaluri bacteria or to remove Edwardsiella ictaluri bacteria, the bacteriophage or variants thereof may be administered to an environment (e.g., a pond) or instrument, or the bacteriophage or variants thereof may be administered to a catfish (e.g., via a feed composition).

Also disclosed herein are isolated polynucleotides which may comprise a portion of the polynucleotide sequence of one of SEQ ID NOs:1-3, or a portion of a reverse complement of one of SEQ ID NOs:1-3. Contemplated polynucleotides include polynucleotides that hybridize to the polynucleotide sequence of one of SEQ ID NOs:1-3, or a portion of a reverse complement of one of SEQ ID NOs:1-3 (e.g., polynucleotide fragments of one of SEQ ID NOs:1-3, or polynucleotide fragments of a reverse complement of one of SEQ ID NOs:1-3, which fragment are at least about 10, 20, 30, 40, or 50 nucleotides in length). Contemplated polynucleotides may comprise contiguous fragments of the disclosed polynucleotide sequences of SEQ ID NOs:1-3 or a reverse complement of one of SEQ ID NOs:1-3. For example, a fragment may comprise at least about 10 contiguous nucleotides of one of SEQ ID NOs:1-3 or a reverse complement of one of SEQ ID NOs:1-3 (or at least about 20, 30, 40, 50, 100, 200, 500, or 1000 contiguous nucleotides of one of SEQ ID NOs:1-3 or a reverse complement of one of SEQ ID NOs:1-3).

In some embodiments, the isolated polynucleotides encode a polypeptide sequence selected from one of SEQ ID NOs:4-106 or a variant polypeptide sequence thereof having at least 95% polypeptide sequence identity to one of SEQ ID NOs:4-106, (e.g., a polypeptide having at least 96%, 97%, 98%, or 99% sequence identity to one of SEQ ID NOs:4-106, respectively, wherein the polynucleotide sequence encodes a polypeptide having a functional or structural activity selected from DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity). Compositions comprising one or more of the disclosed polynucleotides also are contemplated.

Contemplated polynucleotides may include recombinant polynucleotides, for example, recombinant polynucleotides comprising a promoter sequence operably linked to a polynucleotide encoding a polypeptide comprising an amino acid sequence of one of SEQ ID NOs:4-106, or a variant polypeptide sequence thereof. The recombinant polynucleotides optionally may be present in a vector. The recombinant polynucleotides, which optionally may be present in a vector, may be utilized to transform a cell. Further contemplated herein are isolated cells transformed with the recombinant polynucleotides as disclosed herein.

The disclosed polynucleotides may encode one or more polypeptides. Further contemplated herein are isolated polypeptides encoded by the disclosed polynucleotide sequences. For example, the isolated polypeptides may comprise a polypeptide sequence selected from one of SEQ ID NOs:4-106 or a variant polypeptide sequence thereof having at least 95% amino acid sequence identity to one of SEQ ID NOs:4-106, (preferably at least about 96%, 97%, 98%, or 99% amino acid sequence identity to one of SEQ ID NOs:4-106, wherein the polypeptide has a functional or structural activity selected from DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity). Compositions comprising one or more of the disclosed polypeptides also are contemplated herein.

The disclosed polynucleotides may be utilized in methods for producing the encoded polypeptides. The methods may include (a) culturing a cell under conditions suitable for expression of the polypeptide, wherein said cell is transformed with a recombinant polynucleotide, and the recombinant polynucleotide comprises a promoter sequence operably linked to an isolated polynucleotide as disclosed herein (e.g., a polynucleotide encoding a polypeptide comprising a polypeptide sequence of one or SEQ ID NOs:4-106 or a variant polypeptide sequence thereof); and (b) recovering the polypeptide so expressed.

### BRIEF DESCRIPTION OF THE FIGURES

FIG. 1. provides an electron micrograph of phage ΦeiAU, negatively stained with 2% phosphotungstic acid.

FIG. 2. provides a restriction fragment analysis of phages with EcoRI resolved by agarose gel electrophoresis. Arrows show presence of DNA fragments unique to phage ΦeiAU.

FIG. 3. illustrates the effects of CaCl₂ () and MgCl₂ (⋄) on titer of (A) phage ΦeiAU and (B) ΦeiDWF when added to broth cultures of Edw. ictaluri strain 219. Error bars indicate mean (±SD). Bacterial turbidity (X) determined spectrophotometrically at 600 nm

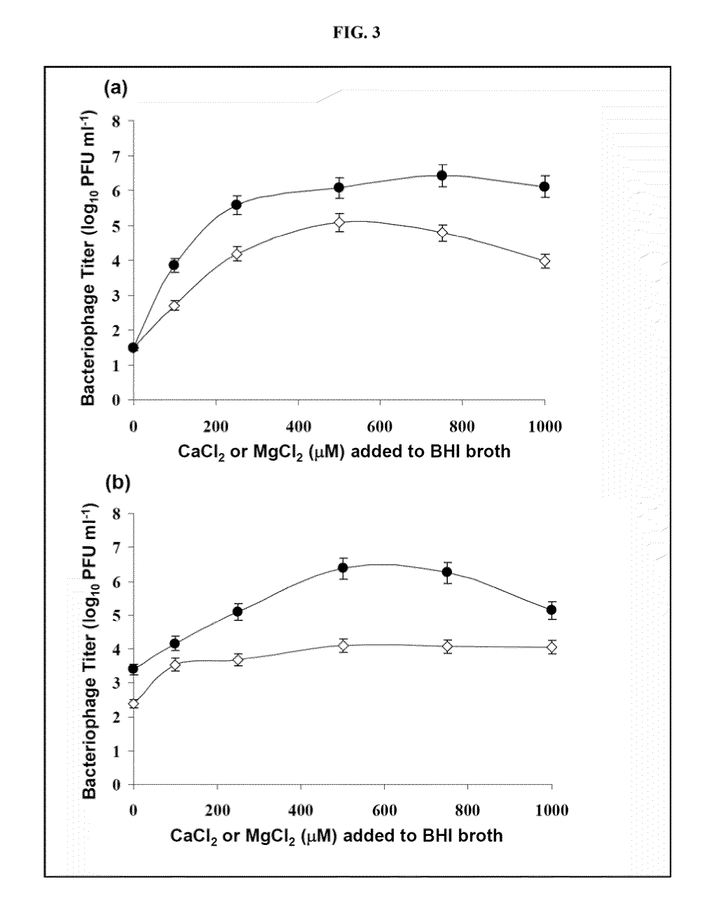

FIG. 4. illustrates the effects of inoculating phage ΦeiDWF into Edw. ictaluri strain 219 cultures in log phase (after 6 h) and stationary phase (after 19 h). Bacterial CFUs in the absence of phage (▪) are compared with the cultures inoculated with phage (O). Cultures were supplemented with 500 μM CaCl₂ and incubated at 30° C. Error bars indicate mean (±SD).

FIG. 5. provides a genomic map of ΦeiAU in comparison to ΦeiMLS and ΦKS7 (Salmonella).

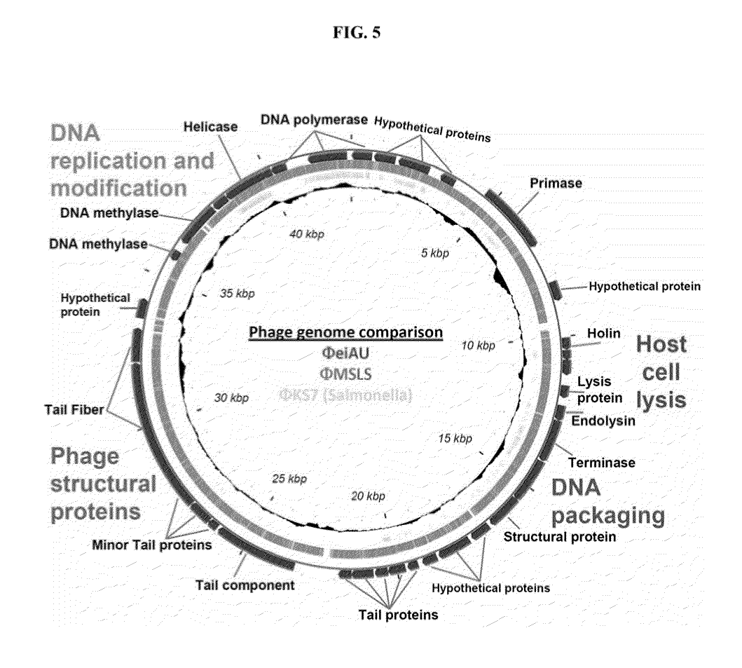

### DETAILED DESCRIPTION

The disclosed subject matter is further described below.

Unless otherwise specified or indicated by context, the terms “a”, “an”, and “the” mean “one or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.”

The term “catfish” refers to a fish belonging to the genus Ictaluri and includes the species Ictaluri punctatus Rafinesque.

The disclosed bacteriophage and variants thereof typically exhibit lytic activity for various species of bacteria, which include Edwardsiella spp. such as Edwardsiella ictaluri. The disclosed bacteriophage and variants thereof characteristically have a circular genome of double-stranded DNA of between 40-45 kb (commonly between 41-43 kb). The disclosed bacteriophage and variants thereof, for example, may have a genome comprising a polynucleotide sequence of one of SEQ ID NOs:1-3 or the reverse complement of a polynucleotide sequence of one of SEQ ID NOs:1-3. The disclosed bacteriophage and variants thereof may have a genome comprising a full-length variant polynucleotide sequence of one of SEQ ID NOs:1-3. The disclosed bacteriophage and variants thereof may include the bacteriophage designated as ΦeiAU and ΦeiDWF. The bacteriophage designated as ΦeiAU was deposited with the American Type Culture Collection (ATCC)®, located at 10801 University Boulevard, Manassas, Va., 20110-2209, USA, on Sep. 15, 2009, and received ATCC® Patent Deposit Designation: PTA-10342.

The term “sample” is used herein in its broadest sense. A sample may comprise a biological sample from an animal (e.g., a biological sample obtained from a catfish) or a sample taken from an environment (e.g., a water sample from a pond or a swabbed surface sample taken from a container or instrument).

As used herein, the term “polynucleotide” refers to a nucleotide polymer having a polynucleotide sequence. A polynucleotide is characterized by a “nucleic acid sequence” or a “polynucleotide sequence,” which terms may be used interchangeably. An “oligonucleotide” refers to a polynucleotide having a relatively short sequence, typically, no more than about 100 nucleotides (more typically no more than about 50 nucleotides, even more typically no more than 20 nucleotides or 10 nucleotides). A polynucleotide as disclosed herein may encode a peptide or polypeptide as disclosed herein. A polynucleotide may be operably linked to a heterologous promoter sequence as a recombinant polynucleotide. “Operably linked” refers to the situation in which a first nucleic acid sequence (e.g., comprising a promoter sequence) is placed in a functional relationship with a second nucleic acid sequence (e.g., encoding a polypeptide). For instance, a promoter is operably linked to a coding sequence if the promoter affects the transcription or expression of the coding sequence. Operably linked DNA sequences may be in close proximity or contiguous and, where necessary to join two protein coding regions, in the same reading frame. A recombinant polynucleotide comprising a polynucleotide operably linked to a promoter sequence may be present in a vector (e.g., a plasmid) which may be utilized to transform a host cell (e.g., where the vector further includes a selectable marker).

The peptides and polypeptides disclosed herein may be described or characterized via their “amino acid sequence.” As used herein, the term “amino acid sequence” refers to an oligopeptide, peptide, polypeptide, or protein sequence, or a fragment of any of these, and to naturally occurring or synthetic molecules. The term “amino acid sequence” may be used interchangeably with the term “polypeptide sequence.” The term “protein” may be used herein interchangeably with the term “polypeptide.” The term “peptide” also may be used herein interchangeably with the term “polypeptide,” however, the term “peptide” typically refers to an amino acid polymer having a relatively low number of amino acid residues (e.g., no more than about 50, 40, 30, 20, 15, or 10 amino acid residues). Generally, the term “polypeptide” refers to an amino acid polymer having a greater number of amino acid residues than a peptide.

The presently disclosed bacteriophage, polynucleotides, and polypeptides may be isolated or substantially purified. The terms “isolated” or “substantially purified” refers to bacteriophage, peptides, or polypeptides that are removed from their natural environment and are isolated or separated, and are at least 75% free, preferably at least 85% free, more preferably at least 95% free, and most preferably at least 99% free from other components with which they are naturally associated. Isolated material may be, for example, heterologous nucleic acid inserted in a vector, non-endogenous nucleic acid contained within a host cell, or any material (e.g., bacteriophage, polynucleotide, or polypeptide) which has been removed from its original environment. Isolated material further includes isolated Edw. ictaluri bacteriophage or particular Edw. ictaluri bacterial isolates, isolated and cultured separately from the environment in which they were originally obtained, where these isolates are present in purified compositions that do not contain any significant amount of other bacteriophage or bacteria. A substantially pure bacteriophage, polynucleotide, or polypeptide is essentially free of any other bacteriophage, polynucleotide, or polypeptide, respectively.

The presently disclosed polypeptides may be expressed by vectors, which may include plasmids, viral vectors, or bacterial vectors. A “plasmid” is an epigenomic circular double-stranded DNA molecule in which foreign nucleic acid encoding a polypeptide may be inserted. A “viral vector” refers to recombinant viral nucleic acid in which foreign nucleic acid may be inserted. Recombinant plasmids and viral vectors typically include cis-acting elements for replication or expression of a foreign nucleic acid encoding a polypeptide. Recombinant attenuated bacteria also may be utilized as vectors.

The present bacteriophage, polynucleotides, and polypeptides may be formulated in a composition which may include a suitable excipient, carrier, or diluent. The compositions may include additional agents such as stabilizers. Suitable stabilizers include, for example, glycerol/EDTA, carbohydrates (such as sorbitol, mannitol, trehalose, starch, sucrose, dextran or glucose), proteins (such as albumin or casein) and protein degradation products (e.g., partially hydrolyzed gelatin). If desired, the formulation may be buffered by methods known in the art, using reagents such as alkali metal phosphates, e.g., sodium hydrogen phosphate, sodium dihydrogen phosphate, potassium hydrogen phosphate and/or potassium dihydrogen phosphate. Further additives which can be used in the present formulation include conventional antioxidants and conventional chelating agents, such as ethylenediamine tetraacetic acid (EDTA).

Edwardsiella ictaluri Bacteriophage and Variants Thereof

The disclosed Edwardsiella ictaluri bacteriophage include, but are not limited to, Edw. ictaluri bacteriophage ΦeiAU and ΦeiDWF. The bacteriophage designated as ΦeiAU was deposited with the ATCC® and received ATCC® Patent Deposit Designation: PTA-10342. Unless otherwise indicated, use of the term “Edwardsiella ictaluri bacteriophage” in this application is intended to include each of these deposited bacteriophage, or mixtures of the two, as well as variant Edwardsiella ictaluri bacteriophage as disclosed herein, or mixtures thereof.

The disclosed Edwardsiella ictaluri bacteriophage exhibit specificity with respect to lysing Edw. ictaluri. The Edw. ictaluri bacteriophage disclosed herein have specific biological activity (e.g., the ability to lyse host Edw. ictaluri bacteria and the ability to produce phage progeny in Edw. ictaluri bacteria). Also contemplated herein are variant Edw. ictaluri bacteriophage, which typically are bacteriophage having minor variation(s) in their genomic sequence or the polypeptides encoded therein while retaining the same general genotypic and phenotypic characteristics as the parent Edw. ictaluri bacteriophage, including the ability to lyse Edw. ictaluri bacteria and produce clear plaques. Other shared phenotypic characteristics are icosahedral heads, non-rigid tails, and tentative classification in the phylogentic family Siphoviridae. Other shared characteristics include an approximate genome size between 40 and 45 kb (commonly between 41 kb and 43 kb), which genome may include open reading frames encoding polypeptides having one or more of the following functional or structural activities: DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity.

Variant Edwardsiella ictaluri bacteriophage may include one or more insertions, deletions, or substitutions in their genomes relative to wild-type Edw. ictaluri bacteriophage (e.g., relative to the genomes of ΦeiAU, ΦeiDWF, or both), while retaining the ability to lyse Edw. ictaluri bacteria. Preferably, variant Edw. ictaluri bacteriophage have a genome that has at least about 95% sequence identity to the genome of ΦeiAU, ΦeiDWF, or both (more preferably at least about 96%, 97%, 98%, or 99% sequence identity to the genome of ΦeiAU, ΦeiDWF, or both). A variant Edw. ictaluri bacteriophage may express variant polypeptides. Preferably, the variant polypeptides expressed by the variant Edw. ictaluri bacteriophage exhibit the biological activity associated with the corresponding wild-type polypeptide (e.g., one of DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity). A variant Edw. ictaluri bacteriophage may include one or more mutations that are silent with respect to a polypeptide encoded by a polynucleotide comprising the one or more mutations. For example, a variant Edw. ictaluri bacteriophage may have genome that is a full-length variant of the genome of ΦeiAU, ΦeiDWF, or both, but nonetheless expresses polypeptides that have identical amino acid sequences to the polypeptides of ΦeiAU, ΦeiDWF, or both, based on degeneracy of the genetic code Variants of Edw. ictaluri bacteriophage include polymorphic variants. Variants of Edw. ictaluri bacteriophage may include bacteriophage that have been passaged (e.g., ΦeiAU or ΦeiDWF which have been passaged on Edw. ictaluri bacteria or chosen strains thereof) and selected for specific phenotypic traits (e.g., modified lytic traits such as larger plaque production, rapid growth, and the like.)

Also contemplated herein are recombinant Edwardsiella ictaluri bacteriophage having modified genotypic or phenotypic characteristics relative to the deposited Edw. ictaluri bacteriophage ΦeiAU, ΦeiDWF, or both. For example, recombinant bacteriophage may include recombinantly designed Edw. ictaluri bacteriophage harboring genes encoding novel phenotypic traits. Such recombinant Edw. ictaluri bacteriophage may be engineered to contain heterologous genes having traits not found in wild-type Edw. ictaluri bacteriophage.

Polynucleotides disclosed herein may be utilized for producing derivative Edwardsiella ictaluri bacteriophage, particularly recombinant Edw. ictaluri bacteriophage. In one embodiment, homologous recombination techniques may be used to introduce homologous sequences encoding alternative proteins, non-functional proteins, or non-coding sequences into the Edw. ictaluri bacteriophage DNA sequence disclosed herein. Such techniques may be utilized to “knock-out” undesirable traits of the Edw. ictaluri bacteriophage or to introduce different and desirable traits. Homologous recombination further may be utilized to introduce or knock-out genes involved in burst size. In particular, homologous recombination may be used to introduce genes which increase the phage burst size.

Production of Edwardsiella ictaluri Bacteriophage

Edwardsiella ictaluri bacteriophage may be produced using a culture system. More specifically, host Edw. ictaluri bacteria may be cultured in batch culture, followed by inoculation of the Edw. ictaluri culture with an appropriate inoculum of Edw. ictaluri bacteriophage. After incubation, the Edw. ictaluri bacteriophage may be harvested and filtered to yield phage progeny suitable for further use. The bacteriophage obtained therefrom may be utilized to prepare compositions comprising active viral particles of Edw. ictaluri bacteriophage capable of lysing Edw. ictaluri bacteria.

The concentration of Edw. ictaluri bacteriophage in a composition may be determined using phage titration protocols. The final concentration of Edw. ictaluri bacteriophage may be adjusted by dilution with buffer to yield a desirable phage titer (e.g., in some embodiments 10⁹-10¹¹ PFU/ml). The resulting Edw. ictaluri bacteriophage composition may be stored (e.g., after freeze- or spray-drying). The stored composition may be reconstituted, and the reconstituted phage titer may be determined using phage titration protocols on host Edw. ictaluri bacteria.

Environmental Control of Edwardsiella ictaluri

Compositions comprising Edwardsiella ictaluri bacteriophage as disclosed herein may be administered to environments to control the growth or viability of Edw. ictaluri. Environments in which Edw. ictaluri bacteriophage is useful to control the growth or viability of Edw. ictaluri include, but are not limited to, aquaculture facilities, ponds, and the like, wherein catfish are raised, including but not limited to catfish otherwise named Ictaluri punctatus Rafinesque. Compositions comprising Edw. ictaluri bacteriophage as disclosed herein also may be administered or applied to instruments utilized in aquaculture facilities wherein catfish are raised in order to prevent the instruments from speading Edw. ictaluri bacteria.

Suitable modes of administration may include, but are not limited to, spraying, hosing, and any other reasonable means of dispersing Edw. ictaluri bacteriophage compositions (either liquid or dry compositions) within the aqueous medium of an aquaculture pond or instrument utilized in raising catfish, in an amount sufficiently high to inhibit the growth or viability of Edw. ictaluri. The administered compositions preferably are useful in preventing the growth or viability of Edw. ictaluri by infecting, lysing, or inactivating Edw. ictaluri present in the environment or present on the instrument. In some embodiments, the Edw. ictaluri bacteriophage may be present in a liquid composition (e.g., a buffered aqueous composition comprising phosphate buffered saline or chlorine-free water), a suspension, or a dry composition (e.g., a lyophilized composition or spray-dried composition).

Edwardsiella ictaluri bacteriophage may be administered at a concentration effective to inhibit the growth or viability of Edw. ictaluri in a particular environment or on a particular surface. In some embodiments, Edw. ictaluri bacteriophage may be administered at an effective concentration of about 10⁷ to 10¹¹ PFU/ml or about 10⁷ to 10¹¹ PFU/cm².

Prevention or Treatment of Infection by Edwardsiella ictaluri

The disclosed bacteriophage also may be utilized for treating or preventing illnesses caused by the bacterium Edwardsiella ictaluri. The methods may include administering an effective amount of an Edw. ictaluri bacteriophage composition for killing Edw. ictaluri or for controlling the growth of Edw. ictaluri to an animal infected by Edw. ictaluri or to an animal at risk for infection by Edw. ictaluri. The composition may be administered to the animal at the site of infection or at a site at risk for infection. The infected animal or animal at risk may be a catfish. The modes of contact include, but are not limited to, spraying or misting the Edw. ictaluri bacteriophage composition on the infected animal or by feeding the animal a composition containing a concentration of Edw. ictaluri bacteriophage sufficiently high to kill or inhibit the growth of Edw. ictaluri.

In some embodiments, the Edw. ictaluri bacteriophage may be present in a liquid composition (e.g., a buffered aqueous composition comprising phosphate buffered saline or chlorine-free water), a suspension, or a dry composition (e.g., a lyophilized composition or spray-dried composition). The composition may be applied to feed to prepare a catfish food composition comprising the bacteriophage (e.g., by spraying a liquid suspension of the bacteriophage on feed, by coating feed with a bacteriophage composition using a commercial feed coating method, or by formulating a feed composition comprising the bacteriophage using “OralJect™” technology, see, e.g., US Published Application Nos. US 2008-0226682 and US 2005-0175724, the contents of which are incorporated by reference in their entireties).

Edwardsiella ictaluri Polynucleotides and Variants Thereof.

Also disclosed herein are polynucleotide molecules of the Edwardsiella ictaluri bacteriophage ΦeiAU and ΦeiDWF. The bacteriophage designated as ΦeiAU was deposited with the ATCC® and received ATCC® Patent Deposit Designation: PTA-10342. Polynucleotide molecules contemplated herein include polydeoxyribonucleotide molecules as well as polyribonucleotide molecules, including modified or unmodified DNA or RNA, which may be double- or single-stranded. Polynucleotides contemplated herein also include modified polynucleotides, such as for example phosphorothioated DNAs or PNAs (Peptide Nucleic Acids). The polynucleotides disclosed herein may be labeled (e.g., by a radiolabel, biotin, fluorescent label, chemiluminescent or colorimetric label), which label may be utilized for diagnostic or tracking and monitoring purposes.

As disclosed herein, variants of Edwardsiella ictaluri bacteriophage polynucleotides may include polynucleotides having at least about 95%, 96%, 97%, 98%, or 99% nucleotide sequence identity relative to a reference polynucleotide molecule (e.g., relative to a polynucleotide having the nucleotide sequence of any of SEQ ID NOs:1-3 or relative to a polynucleotide having a portion of the nucleotide sequence of any of SEQ ID NOs:1-3). “Percentage sequence identity” may be determined by aligning two sequences using the Basic Local Alignment Search Tool available at the NBCI website (e.g., “bl2seq” as described in Tatiana A. Tatusova, Thomas L. Madden (1999), “Blast 2 sequences—a new tool for comparing protein and nucleotide sequences”, FEMS Microbiol Lett. 174:247-250)).

Variant polynucleotide molecules may include fragments of the full-length polynucleotides disclosed herein. Techniques for generating polynucleotide fragments may include, but are not limited to, chemical synthesis and restriction digests. A fragment comprises or consists of a contiguous portion of a nucleotide sequence of the full-length polynucleotide. For example, a fragment may comprise or consist of at least a 10, 20, 30, 40, 50, 60, 70, 80, 90, or 100 contiguous nucleotide sequence of a full-length polynucleotide. In some embodiments, a fragment of a full-length polynucleotide may comprise or consist of a 10-100 contiguous nucleotide sequence of any of SEQ ID NOs:1-3 or the reverse complement thereof. A fragment may include a 5′-terminal truncation, a 3′-terminal truncation, or both, with respect to a reference full-length polynucleotide.

Variants of Edwardsiella ictaluri bacteriophage polynucleotides described herein may encode polypeptides have one or more functional or structural activities exhibited by a polypeptide encoded by a reference polynucleotide (e.g., a functional or structural activity of a polypeptide encoded by a polynucleotide sequence present within one of SEQ ID NOs:1-3, such as DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity).

Edwardsiella ictaluri Polypeptides and Variants Thereof.

Also disclosed herein are polypeptides encoded by the genomes of the isolated Edwardsiella ictaluri bacteriophage ΦeiAU and ΦeiDWF. The bacteriophage designated as ΦeiAU was deposited with the ATCC® and received ATCC® Patent Deposit Designation: PTA-10342. Contemplated polypeptides may include polypeptides having a functional or structural activity selected from, but not limited to, DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity.

Contemplated polypeptides include molecules having an amino acid sequence encoded by the disclosed polynucleotides. The disclosed polypeptides included proteins, peptides and fragments thereof (functional or non-functional) encoded by Edw. ictaluri bacteriophage polynucleotides. Polypeptides may comprise or consist of, antigenic or immunogenic polypeptides, including antigenic or immunogenic polypeptide fragments.

Also contemplated are variant polypeptide molecules as disclosed herein. As used herein, a “variant polypeptide” is a polypeptide molecule having an amino acid sequence that differs from a reference polypeptide molecule. A variant may have one or more insertions, deletions, or substitutions of an amino acid residue relative to a reference polypeptide molecule. For example, a variant polypeptide may have one or more insertions, deletions, or substitutions of at least one amino acid residue relative to the presently disclosed DNA polymerase proteins, Primase protein, Holin protein, Lysis protein, Endolysin protein, Terminase protein, Structural proteins, Tail proteins, DNA methylase protein, and Helicase protein. (See, e.g., the polypeptides encoded by the polynucleotides of SEQ ID NOs:1-3, the polypeptides encoded by the reverse complement of the polynucleotides of SEQ ID NOs:1-3, and the polypeptides of SEQ ID NOs:4-106).

Variants of Edwardsiella ictaluri bacteriophage polypeptides may include polypeptides having at least about 95%, 96%, 97%, 98%, or 99%, amino acid sequence identity relative to a reference polypeptide molecule (e.g., relative to a polypeptide having the amino acid sequence of any of SEQ ID NOs:4-106). “Percentage sequence identity” may be determined by aligning two sequences using the Basic Local Alignment Search Tool available at the NBCI website (e.g., “bl2seq” as described in Tatiana A. Tatusova, Thomas L. Madden (1999), “Blast 2 sequences—a new tool for comparing protein and nucleotide sequences”, FEMS Microbiol Lett. 174:247-250)).

Variant polypeptide molecules may include fragments of the full-length polypeptides disclosed herein. Techniques for generating polypeptide fragments may include, but are not limited to, chemical synthesis and enzymatic digests. A fragment of a full-length reference polypeptide comprises or consists of a contiguous portion of an amino acid sequence of the full-length polypeptide. For example, a fragment may comprise or consist of at least a 10, 20, 30, 40, 50, 60, 70, 80, 90, or 100 contiguous amino acid sequence of a full-length polypeptide. In some embodiments, a fragment of a full-length polypeptide may comprise or consist of a 10-100 contiguous amino acid sequence of any of SEQ ID NOs:4-106. A fragment may include an N-terminal truncation, a C-terminal truncation, or both, with respect to a reference full-length polypeptide.

Variants of Edwardsiella ictaluri bacteriophage polypeptides described herein may have one or functional or structural activities exhibited by a reference polypeptide (e.g., DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity).

Antibodies Against Edwardsiella ictaluri Polypeptides

Antibodies and antigen-binding fragments thereof that bind to the disclosed Edwardsiella ictaluri polypeptides also are contemplated herein (e.g., Edw. ictaluri bacteriophage polypeptides as disclosed herein). The term “antibody” as used herein refers to an immunoglobulin molecule or an immunologically active portion thereof (i.e., an antigen-binding portion). As used herein, the term “antibody” refers to a protein comprising at least one, and preferably two, heavy (H) chain variable regions (abbreviated as VH), and at least one and preferably two light (L) chain variable regions (abbreviated as VL). The VH and VL regions can be further subdivided into regions of hypervariability, termed “complementarity determining regions” (“CDR”), interspersed with regions that are more conserved, termed “framework regions” (FR). “An antigen-binding” refers to one or more fragments of a full-length antibody that retain the ability to specifically bind to the antigen (e.g., Edw. ictaluri bacteriophage polypeptides as disclosed herein). Examples of antigen-binding fragments of the disclosed antibodies include, but are not limited to: (i) an Fab fragment or a monovalent fragment consisting of the VL, VH, CL and CH1 domains; (ii) an F(ab')₂ fragment or a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment (Ward e.g., (1989) Nature 341:544 546), which consists of a VH domain; and (vi) an isolated complementarity determining region (CDR). Even though the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or “scFv.”

The disclosed antibodies can be full-length (e.g., an IgG (e.g., an IgG1, IgG2, IgG3, IgG4), IgM, IgA (e.g., IgA1, IgA2), IgD, and IgE) or can include only an antigen-binding fragment (e.g., a Fab, F(ab′)₂ or scFV fragment, or one or more CDRs). The antibodies disclosed herein may be a polyclonal or monoclonal antibodies. The disclosed antibodies may be monospecific, (e.g., a monoclonal antibody, or an antigen-binding fragment thereof), or may be multispecific (e.g., bispecific recombinant diabodies). In some embodiments, the antibody can be recombinantly produced (e.g., produced by phage display or by combinatorial methods). In some embodiments, the antibodies (or fragments thereof) are recombinant or modified antibodies (e.g., a chimeric or an in vitro generated antibody).

Use of Edwardsiella ictaluri Polynucleotides, Polypeptides, and Antibodies

The Edwardsiella ictaluri polynucleotides and the encoded polypeptides disclosed herein may be utilized to prevent or inhibit the growth of Edw. ictaluri. For example, Edw. ictaluri bacteriophage lytic enzymes or the polynucleotides that encode these enzymes may be utilized to prevent or inhibit the growth of Edw. ictaluri through cell wall lysis. Compositions comprising Edw. ictaluri polynucleotides and the encoded polypeptides may be administered to environments colonized by Edw. ictaluri or at risk for colonization by Edw. ictaluri. Composition comprising Edw. ictaluri polynucleotides and the encoded polypeptides further may be administered to animals infected by Edw. ictaluri or at risk for infection by Edw. ictaluri in order to treat or prevent infection.

Edwardsiella ictaluri bacteriophage polynucleotides or antibodies against Edw. ictaluri bacteriophage polypeptides may be utilized to detect the presence of Edw. ictaluri bacteriophage. For example, a polynucleotide fragment of at least about 10, 15, or 20 nucleotides in length may be utilized as a probe for identifying the presence of Edw. ictaluri bacteriophage in a sample (e.g., using stringent hybridization techniques as known in the art). Pairs of polynucleotide fragments of at least about 10, 15, or 20 nucleotides in length may be utilized as primers for identifying the presence of Edw. ictaluri bacteriophage in a sample using PCR amplification techniques. Antibodies against Edw. ictaluri bacteriophage polypeptides further may be utilized in immunoassays for detecting Edw. ictaluri bacteriophage in a sample. Polynucleotide probes and antibodies may be conjugated to labels which include, but are not limited to, radiolabels, biotin, fluorescent labels, chemiluminescent or calorimetric labels.

Identifying Edwardsiella ictaluri in Samples

The Edwardsiella ictaluri bacteriophage disclosed herein further may be utilized for identifying Edw. ictaluri or isolates thereof in a sample. For example, the Edw. ictaluri bacteriophage disclosed herein may be contacted with a sample comprising unknown bacteria, whereby if the bacteriophage lyse the unknown bacteria in the sample, Edw. ictaluri or isolates thereof which are subject to lysis by the bacteriophage are identified. Edwardsiella ictaluri bacteriophage may be combined with other bacteriophage in the identification method to further identify or characterize bacteria in the sample.

### EXAMPLES

The following Examples are illustrative and are not intended to limit the scope of the claimed subject matter.

### Example 1

### Identification and Characterization of Bacteriophages Specific to the Catfish Pathogen, Edwardsiella ictaluri

Reference is made to Walakira et al., “Identification and characterization of bacteriophages specific to the catfish pathogen, Edwardsiella ictaluri,” J. Appl. Micro, 105(6):2133-2142, available online Oct. 21, 2008, the content of which is incorporated herein by reference in its entirety.

Summary

Two bacteriophages were isolated that infect Edwardsiella ictaluri and have been named ΦeiAU and ΦeiDWF. Both phage produce clear plaques, have icosahedral heads with a non-rigid tail, and are tentatively classified as Siphoviridae. Phages ΦeiAU and ΦeiDWF are dsDNA viruses with approximate genome sizes between 40 and 45 kb. The addition of 500 μM CaCl₂ enhanced phage titers. Both phages have a latent period of 40 min and an estimated burst size of 270. Every Edw. ictaluri strain tested was susceptible to phage infection with variable plaquing efficiencies and with no evidence of lysogeny, with no plaques detected on other bacterial species. This is the first report of bacteriophages specific to Edw. ictaluri, an important fish pathogen affecting farm-raised channel catfish. Initial characterization of these bacteriophages has demonstrated their potential use as biotherapeutic and diagnostic agents associated with ESC.

### Methods and Materials

Bacteria and media. Twenty five bacterial isolates from the Southern Cooperative Fish Disease laboratory with the Department of Fisheries and Allied Aquacultures, College of Veterinary Medicine Department of Pathobiology, Auburn University and ATCC collections were used in this study (Table 1).

TABLE 1
Efficiency of plaquing (EOP) of ΦeiAU and ΦeiDWF on Edw. ictaluri strains and
other bacterial species isolated and collected from different locations.

| | EOP¹ | | |
|---|---|---|---|
| Bacteria | ΦeiAU | ΦeiDWF | Source² |
| Edwardsiella ictaluri strains | | | |
| ATCC 33202 | 106 | 223.1 | Catfish, Mississippi |
| AL93-92 | 61.1 | 77.9 | Catfish, Alabama |
| AU98-25-42A | 76.4 | 157.4 | Catfish, Alabama |
| 195 | 27.3 | 33.8 | Catfish, Alabama |
| 196³ | 10⁻⁴ to 10⁻⁷ | 10⁻⁴ to 10⁻⁷ | Catfish, Alabama |
| 218 | 112.5 | 131.8 | Catfish, Mississippi |
| 219 | 100 | 100 | Catfish, Alabama |
| S97 773 | 106.9 | 66.8 | Catfish, Alabama |
| RE-33 | 150 | 306.1 | AUFDL |
| C91-162³ | 10⁻⁴ to 10⁻⁷ | 10⁻⁴ to 10⁻⁷ | AUCVM |
| R4383³ | 10⁻⁴ to 10⁻⁷ | 10⁻⁴ to 10⁻⁷ | AUCVM |
| Aeromonas hydrophila GA-06-05 | — | — | Catfish, Georgia |
| Citrobacter freundii ATCC 8090 | — | — | ATCC |
| Edwardsiella tarda AL 9338 | — | — | Catfish, Alabama |
| Enterobacter aerogenes CDC 65966 | — | — | ATCC |
| Flavobacterium columnare ALG 530 | — | — | Catfish, Alabama |
| Flavobacterium columnare AL-04-35 | — | — | Tilapia, Alabama |
| Flavobacterium columnare CR-04-02 | — | — | Tilapia, Costa Rica |
| Flavobacterium columnare SC-04-04 | — | — | Carp, South Carolina |
| Flavobacterium columnare TN-02-01 | — | — | Catfish, Tennessee |
| Klebsiella pneumoniae ATCC 25953 | — | — | ATCC |
| Proteus mirabilis | — | — | AUFDL |
| Salmonella enterica ATCC 12324 | — | — | ATCC |
| Yersinia ruckeri biotype I MO-06-08 | — | — | Trout, Missouri |
| Yersinia ruckeri biotype II SC-04-13 | — | — | Trout, South Carolina |

¹The EOP for each phage was determined as a ratio of PFU ml⁻¹ for each strain relative to tha obtained from Edw. ictaluri strain 219, determined after 12 h of incubation at 30° C.
²AUCVM, Auburn University College of Veterinary Medicine (Department of Pathobiology) AUFDL, Auburn University Fish Diagnostic Laboratory.
³Quantification of EOP was difficult in these strains due to a very small plaque size (<1 mm)

With the exception of Edw. ictaluri strain RE-33, Edw. ictaluri strain 84383, Edw. ictaluri strain C91-162, Citrobacter freundii strain ATCC 8090, Klebsiella pnuemoniae ATCC 25953, Proteus mirabilis and Salmonella enterica ATCC 12324, all isolates were obtained from disease cases submitted from farms in various geographical locations. The Edw. ictaluri strain 219 was used for the general characterization of the bacteriophages. The remaining isolates were used to test for host range of the phages.

Flavobacterium columnare isolates were grown in Hsu-Shotts medium (Bullock et al. 1986) and the remaining bacterial isolates were propagated on brain heart infusion (BHI) media (Difco, Sparks Md., USA) at 30° C., and stored in their respective broth at −80° C. in 10% glycerol. Biochemical tests were performed using protocols described by the AFS-FHS Blue Book (American Fishery Society-Fish Health Section, Bethesda, Md., USA). Various assays (e.g., Gram stain, cytochrome oxidase, indole production, hydrogen sulfide production, and motility) were performed on Edw. ictaluri strains grown on Remel BHI agar (Fisher Scientific, Lenexa, Kans., USA).

Enrichment and isolation of bacteriophages. Water samples were collected from eight commercial catfish ponds that had recently been diagnosed with ESC (at least 3 L were collected for processing from each pond). Algal cells and debris were pelleted by centrifugation at 3,600 g for 30 min. Following removal of most cells, viruses within the supernatant were concentrated using 30-100 kDa Amicon Centricon Plus-70 ultrafiltration membranes (Millipore, Billerica, Mass., USA) while centrifuging at 3,600 g for 15 min. Samples were subsequently sterilized through 0.22 μm PVDF filters (Millipore, Bedford, Mass., USA).

Bacteriophages specific to Edw. ictaluri were enriched as described by O'Flynn et al. (2004) with some modifications. Pond concentrates (˜5 ml) were added to 30 ml log-phase Edw. ictaluri strain 219 cultures (3.1×10⁷ CFU ml⁻¹) and grown overnight at 30° C. with shaking (150 rpm). One percent chloroform (Fisher Scientific, Sair Lawn, N.J., USA) was added to 1.5 ml of culture and subjected to centrifugation at 3,600 g for 10 min at 4° C. The supernatant (1 ml) was then concentrated down to 100 μl using ultrafiltration filters while centrifuging at 3,600 g for 10 min. The presence of lytic phages was tested by spotting 5 μl of filtrate onto a lawn of Edw. ictaluri grown at 30° C. on BHI agar.

In addition, samples from diseased catfish reared at E.W Shell Fisheries Center in Auburn, Ala., were also analyzed for presence of bacteriophages. Kidney and liver samples were homogenized and spread onto BHI agar for isolation Edw. ictaluri and identification of phage plaques. Identified plaques were inoculated into a log-phase culture of Edw. ictaluri, and the phage lysate stored at −80° C. until further analysis (J. Plumb, personal communication).

Bacteriophages were triple purified from isolated plaques using the soft agar overlay method (Adam 1959). A mixture of 100 μl of viral concentrate and 200 μl of log phase Edw. ictaluri strain 219 were added to 5 ml of molten 0.7% BHI agar (maintained at 35° C.) and then poured over BHI agar plates. Plates were incubated overnight at 30° C. to allow for plaque formation. Isolated plaques were picked using sterile wooden toothpicks into a 5 ml log-phase Edw. ictaluri broth culture and incubated at 30° C. with shaking (150 rpm) for 8 h. Purified phages were then stored in SM buffer [100 mM NaCl, 8 mM MgSO₄, 50 mM Tris-HCl (pH 7.5)], and 0.002% (w/v) gelatin at 4° C. with the addition of 7% dimethyl sulfoxide (DMSO) at −80° C.

Phage stocks used in this study were prepared using soft agar overlays as described previously (Su et al. 1998). A confluently lysed plate was flooded with 7 ml of SM buffer and incubated at 30° C. with shaking at 60 rpm for 4 h. Phage suspensions were then centrifuged at 3,600 g for 10 min to remove cells and debris, and the supernatant was filter-sterilized through a 0.22 μm PVDF filter. Plaque assays as described by Adams (1959) were performed to determine the titer of a phage stock. After a 10-fold dilution of the phage stock, 10 μl of each dilution were spotted on a lawn of Edw. ictaluri and then incubated overnight at 30° C. to determine the number of plaque forming units (PFU). Stock samples were stored at −80° C. in 7% DMSO for further studies.

Electron microscopy. Five microliters of CsCl-purified phage (10¹² PFU ml⁻¹) were applied to 300 mesh formvar- and carbon-coated copper grids (Electron Microscopy Services, Hatfield, Pa., USA). Excess liquid was removed after 15 min and each sample was negatively stained with 2% phosphotungstic acid. Using a Zeiss EM10 transmission electron microscope (Zeiss/LEO, Oberkochen, Germany), the grids were examined at various magnifications to determine the morphology and size of each phage.

Isolation and restriction of bacteriophage nucleic acids. Contaminating host chromosomal DNA was removed from a phage stock by adding 250 units of Benzonase® (Novagen, Inc., Madison, Wis., USA) and incubating overnight at 37° C. Benzonase was inactivated by addition of 10 mM EDTA and heating at 70° C. for 10 min. Phage protein coats were degraded using 1 mg ml⁻¹ proteinase K (Novagen, Inc., Madison, Wis., USA) and 1% sodium dodecyl sulphate and incubated at 37° C. for 2 h. Proteins were removed by phenol-chloroform extraction, and phage DNA was ethanol precipitated and resuspended in 75 μl nuclease free, deionized and distilled water. Bacteriophage DNA was digested with EcoRI for at least 3 h at 37° C., and resolved by agarose gel electrophoresis on 1% agarose gels at 70V for 3 h. Gels were stained with ethidium bromide and visualized with an AlphaImager® HP gel documentation system (Alpha Innotech Corporation, San Leandro, Calif., USA).

Effects of temperature, Ca and Mg on bacteriophage replication. The effects of calcium, magnesium and temperature were examined to determine optimal conditions for the infectivity of both phages. To monitor the effect of temperature on phage multiplication, a log-phase Edw. ictaluri strain 219 (10⁶ CFU ml⁻¹) culture in BHI broth was infected with approximately 10⁴ PFU ml⁻¹ and samples were incubated at temperatures between 17-37° C. for 5 h. Phage lysates were subjected to centrifugation at 16,100 g for 5 min, filter-sterilized through 0.22 μm PVDF filters and then quantified by spotting serial dilutions onto Edw. ictaluri lawns.

An overnight bacterial culture was sub-cultured into 50 ml BHI broth prior to adding phage at a multiplicity of infection (MOI) of 0.1 (phage:host). The effect of CaCl₂ and/or MgCl₂ (ranging from 0 to 1 mM added to BHI broth) on phage titers was determined. Samples were assayed to determine the PFU ml⁻¹ and the bacterial culture turbidity (OD₆₀₀) after eight hours of incubation at 30° C. Statistical analysis of the differences between treatment means for each phage was assessed using a one-way analysis of variation (ANOVA) at a 5% significant level.

One-step growth. A one-step growth experiment was conducted based on methods described by Adams (1959) with modifications. Duplicates of ΦeiDWF and ΦeiAU were separately added to Edw. ictaluri strain 219 broth cultures with 1 mM potassium cyanide (KCN), at a MOI of 0.1. Samples were then incubated at 30° C. for 10 min to allow phage-bacteria adsorption. Cells were pelleted by centrifugation (20,000 g, for 2 min at 4° C.), resuspended in fresh BHI broth, diluted 10⁵-fold and incubated at 30° C. while shaking. Aliquots were removed at 5 min intervals and PFU determined by the soft agar overlay method described above.

Phage lysis of host cells. A time course experiment was used to determine the phage-induced lysis of host cells as described by O'Flynn et al. (2004) with slight modifications. An overnight culture of Edw. ictaluri strain 219 was inoculated (1% v/v) into BHI broth media with 500 μM CaCl₂ then incubated at 30° C. while shaking. After 7 h, triplicate samples of ΦeiDWF and ΦeiAU were separately introduced into log phase Edw. ictaluri strain 219 cultures (approx. 10⁶ CFU ml⁻¹) at a MOI of 0.1, and none in the control cultures. Samples were drawn every hour and plated for CFU ml⁻¹. Both phages were also added to stationary phase Edw. ictaluri strain 219 cultures (approx. 10¹⁰ CFU ml⁻¹) at a MOI of 0.1 and incubated at 30° C.

Host range determination. The host range of both phages was assessed on a range of Gram-negative bacteria (Table 1). Susceptibility of various bacterial isolates was tested using the drop-on-lawn technique (Zimmer et al. 2002). The efficiency of plaquing (EOP) was then determined using Edw. ictaluri strain 219 as a reference strain. The EOP of a phage on a given strain of Edw. ictaluri was expressed as the ratio of the PFU ml⁻¹ of a given host strain relative to that observed on Edw. ictaluri strain 219.

Prophage induction. All isolates of Edw. ictaluri used in the host range study were tested for lysogenic phage using a method described by Fortier and Moineau (2007) with modifications. An overnight culture of Edw. ictaluri was sub-cultured (3% v/v) in fresh BHI broth and incubated at 30° C. with shaking until cultures reached an OD₆₀₀ of 0.100. To a 5 ml of Edw. ictaluri culture, Mitomycin C (Sigma-Aldrich, St Louis, Mo., USA) was added to a final concentration of 1 μg ml⁻¹ and then incubated for 30 min. Cells were pelleted by centrifugation at 3,700 g for 5 min, resuspended in fresh BHI broth and incubated for 5 h at 30° C. with shaking (150 rpm). Samples were then centrifuged at 3,700 g for 5 min and 10 μl of supernatant spot assayed for presence of phage against all tested strains.

Results

Isolation of bacteriophages. From aquaculture pond enrichments, one out of eight pond enrichments had evidence of Edw. ictaluri phage plaques. Sixteen phages were double purified from samples collected from Dean Wilson Farms in western Alabama, and six phages were double purified from samples obtained from an infected catfish kidney tissue from the E.W Shell Fisheries Center in Auburn, Ala. Phages isolated from the aquaculture pond had plaques ranging from 0.5 to 11 mm in size and those isolated from infected catfish kidney tissue ranged from 4 to 7 mm. Both phages produced clear plaques on a lawn of host bacteria. No differences were observed in the restriction fragment profiles between the 16 separate phage isolates from the aquaculture pond, or between the six phage isolates from the catfish kidney tissue (data not shown), and one representative phage was chosen from the aquaculture pond enrichment (ΦeiDWF) and the catfish kidney tissue (ΦeiAU) for further study.

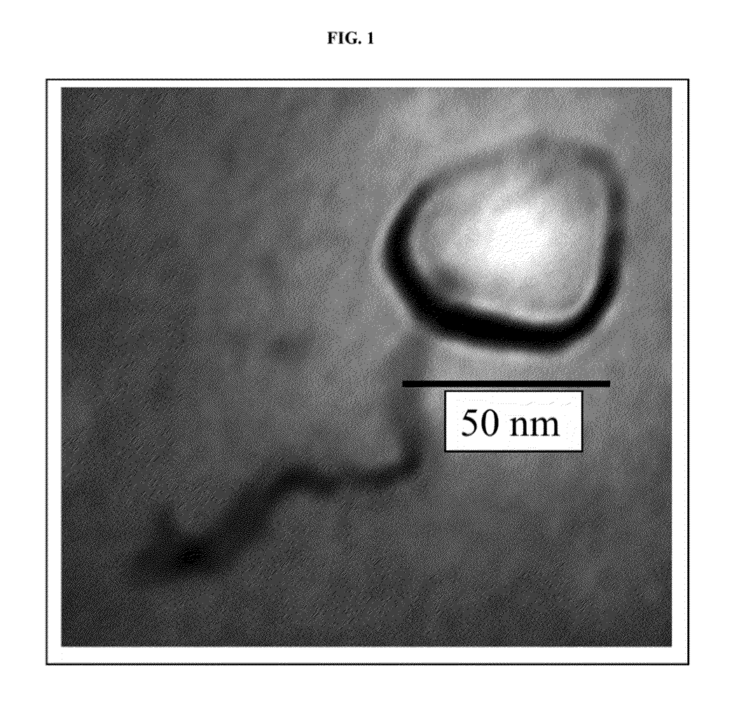

Size and morphology of bacteriophages. Electron microscopy revealed similarity in morphology between ΦeiAU and ΦeiDWF (ΦeiAU shown in FIG. 1). Both have an icosahedral shaped head, 50 nm in diameter, and a non-rigid tail. Tail lengths of ΦeiAU and ΦeiDWF are both approximately 100 nm. Based on the morphology and the rules provided by International Committee on Taxonomy of Viruses (ICTV, Bethesda Md., USA) both phages are tentatively placed in the Siphoviridae family (Murphy et al. 1995, Nelson 2004).

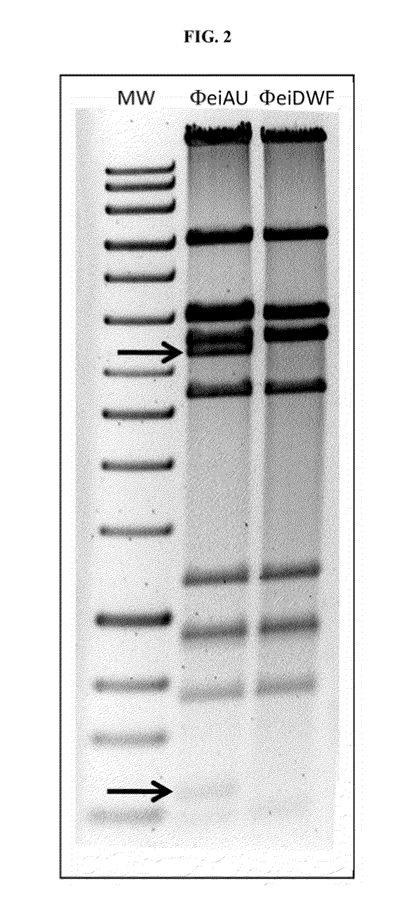

Bacteriophage nucleic acid restriction fragment analysis. Phage nucleic acids were not digested by exonuclease I, indicating that the phages are double-stranded DNA phages. Restriction endonuclease digestion of ΦeiAU and ΦeiDWF with EcoRI showed many bands in common (FIG. 2); however, phage ΦeiAU had two additional restriction fragments compared to ΦeiDWF (FIG. 2). Their dsDNA genome sizes are approximately 40 kb (ΦeiDWF) and 45 kb (ΦeiAU).

Effects of temperature and metal cations on phage titer. Infection of Edw. ictaluri by ΦeiAU and ΦeiDWF is dependent upon temperature and the presence of calcium and magnesium salts. The optimal temperature for growth of Edw. ictaluri (25-30° C.) also supports rapid replication of these phages. Over three orders of magnitude decrease were observed in PFU ml⁻¹ when the temperature was lowered to 20° C. Similarly low phage titers were obtained at temperatures higher than of 30° C. (data not shown).

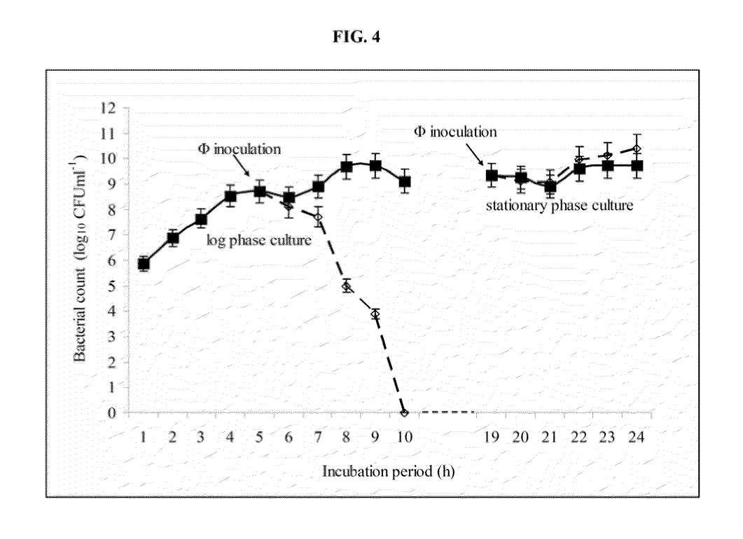

Phage titers of both ΦeiAU and ΦeiDWF are increased by the addition of calcium and magnesium salts to BHI broth. The addition of calcium to BHI broth increased phage titers for both ΦeiAU and ΦeiDWF by several orders of magnitude in a dose-dependent manner (FIG. 4). It is important to note that the initial phage inoculum in these experiments was approximately identical (˜1×10⁴ PFU ml⁻¹) for ΦeiAU and ΦeiDWF, yet in the absence of supplemental calcium or magnesium the phage titer of ΦeiAU decreased substantially during the five hours of incubation. The optimal range observed for calcium and magnesium is 500-750 μM at which a substantial decrease in bacterial turbidity was observed with a corresponding increase in phage titers. The effects of supplementing CaCl₂ and MgCl₂ (both standardized at 500 μM) showed a significant increase (P<0.05: Dunnet's test) of approximately one to two orders of magnitude relative to the titers obtained with addition of CaCl₂ alone for ΦeiAU and ΦeiDWF, respectively (data not shown).

Burst size and latent period. The one-step growth curve was performed for both ΦeiAU and ΦeiDWF, revealing an identical latent period for these bacteriophages of approximately 40 min and with an average burst size estimated to be 270 viral particles (ΦeiAU and ΦeiDWF) per host cell. These calculations were based on the ratio of mean yield of phage particles liberated to the mean phage particles that infected the bacterial cells in the latent period.

Kinetics of phage-induced lysis. Within six hours of incubation of either phage into a log-phase Edw. ictaluri strain 219 culture (about 10⁶ CFU ml⁻¹ at the time of inoculation) the CFU were reduced to below detectable levels (ΦeiDWF shown in FIG. 4). During this six hour period, bacterial cultures with phage rapidly cleared while the controls remained turbid. The loss of turbidity and drop in CFU ml⁻¹ due to both phages was attained within the same incubation period. Furthermore, when ΦeiDWF was inoculated into stationary-phase Edw. ictaluri strain 219 cultures, no clearance of the bacterial culture was observed throughout the incubation period (FIG. 4). However, when the phage inoculated, stationary phase culture of Edw. ictaluri was pelleted by centrifugation and resuspended in fresh medium, the culture turbidity rapidly cleared and the phage titers increased by several orders of magnitude (data not shown).

Host specificity of phages. Both ΦeiAU and ΦeiDWF infected every Edw. ictaluri strain that was tested (Table 1). Clear plaques were produced on all strains except on Edw. ictaluri strain AL93-92 and AL98-25-42A which had a mixture of opaque and clear plaques. Plaque size ranged from 0.5 to 4 mm. However, small pin-point plaques were produced on Edw. ictaluri strains 196, C91-162 and R4383 that appeared only when high phage titers (>10⁶ PFU ml⁻¹) were used. Variable ranges in EOP (˜10′ to 300% relative to strain 219) were observed among Edw. ictaluri strains. Both phages produced high EOP values (>50% relative to strain 219) with Edw. ictaluri strains 218, S97-773, RE-33, AL93-92 AU-98-25-42A and 195 while low values (EOP<10⁻⁴) where observed with Edw. ictaluri strains 196, C91-162, and R4383. None of the other bacterial species tested were observed to have any evidence of phage plaques including the closely related Edw. tarda.

Prophage induction. Mitomycin C was added to cultures of 11 different Edw. ictaluri strains in log-phase to induce any prophage(s) existing in the host cells (Goh et al. 2005). An increase in turbidity was observed in all cultures tested during the 5 h of incubation. No plaques were observed on any strain of Edw. ictaluri indicating the absence of temperate phages in the Edw. ictaluri isolates used in this study.

Discussion

Bacteriophages specific to Edw. ictaluri were isolated from aquaculture ponds with outbreaks of ESC. This finding suggests that Edw. ictaluri-specific phages exist in aquaculture ponds and may contribute to some degree in lessening the severity or persistence of ESC outbreaks. Since Edw. ictaluri is also reported to survive in water and pond bottom sediments for several hours (Inglis et al. 1993, Hawke et al. 1998, Plumb 1999) there is reason to suspect that both Edw. ictaluri and its respective phages may persist in aquaculture ponds. This finding is in accordance with the idea that bacteriophages are ubiquitous in the environments inhabited by their respective host(s) (d'Herelle 1926, Adams 1959). Therefore, catfish pond waters and diseased fish are a good source for discovery of phages specific to Edw. ictaluri. In addition, the gut microbiota of channel catfish with ESC is an as-yet-unexplored environment in which to identify bacteriophages specific to Edw. ictaluri.

The phages described in this study were isolated from samples that differed both temporally and spatially, however electron microscopy revealed similar morphotypes, classified as Siphoviridae. Furthermore, restriction digests using EcoRI and EcoRV showed similar but unique patterns, suggesting that ΦeiAU and ΦeiDWF may have genetic loci in common. Another Edw. ictaluri-infective phage, ΦMSLS-1, has been recently isolated from aquaculture ponds in Mississippi with a history of ESC infection (Dr T. Welch and Dr G. Waldbeiser, USDA, personal communication). A comparison of the EcoRV restriction profiles of ΦDMSLS-1, ΦeiAU, and ΦeiDWF showed a majority of restriction fragments in common with only a few unique restriction fragments (data not shown). Preliminary genome sequences from ΦMSLS-1, ΦeiAU, and ΦeiDW also support this conclusion (data not shown).

The primary factors influencing in vitro phage infectivity for Edw. ictaluri were temperature (optimal 22-33° C.), metal cations (especially calcium), and the host growth stage. Phage reproduction is dependent on the physiological state of the bacterial host (Adams 1959, Taddei & Paepe 2006, Poranen et al. 2006). Normally, ESC epizootics occur when temperatures range from 22 to 28° C. and are characterized by acute infections and high mortalities within young-of-the-year catfish fingerlings (Francis-Floyd et al. 1987, Tucker & Robinson 1990, Durborrow et al. 1991, Inglis et al. 1993). Temperature influences the metabolic activities of the host but also accelerates the adsorption rate of phage (Adams 1959, Fujimura & Kaesberg 1962, Moldovan et al. 2007). Moldovan et al. (2007) demonstrated an increase in adsorption rate (approx. 30 times) when the temperature rose from 4 to 40° C. when λ phage was incubated with E. coli strain Ymel. The role of Ca²⁺ and Mg²⁺ ions in phage-host interaction may be in the adsorption, penetration processes or in other growth stages of phage (d'Herrelle 1926, Luria & Steiner 1954, Adams 1959, Moldovan et al. 2007). It is also postulated that Ca²⁺ ions may increase the concentration of phage particles at the host surface or alter the structure of a cell surface receptor thereby increasing accessibility to the receptor molecules or transfer of phage nucleic acids (Watanabe & Takesue 1972, Russell et al. 1988). The observation that ΦeiAU had a substantial decrease (˜1000-fold) in titer after incubation with Edw. ictaluri in the absence of supplemental calcium or magnesium, yet could productively infect Edw. ictaluri when calcium or magnesium were added to the medium, supports the hypothesis that ΦeiAU (and to a lesser degree, ΦeiDWF) adsorbs to an Edw. ictaluri surface receptor that permits productive infection (e.g., phage nucleic acid transfer) in the presence of metal cations. Alternatively, divalent metal cations could be integral to the structural integrity of the bacteriophage(s). Interestingly, results show that the optimal calcium concentration for phage replication (500 μM) is equivalent to 50 ppm Ca²⁺ recommended in commercial catfish ponds (Tucker and Robinson, 1990). Incidentally, pond environments have varying degrees of Ca²⁺ hence phage infectivity in aquaculture ponds might be influenced by water hardness. Future studies will address the mechanism(s) of metal cation-induced increases in phage titers, and the role of metal cations in phage biological control of ESC in aquaculture ponds.

Both phages are specific to Edw. ictaluri strains without generating plaques on any other bacterial species. Although Edw. tarda is reported to be closely related to Edw. ictaluri (Zhang & Arias, 2006), it was not susceptible to phages evaluated in this study. Because of their specificity, both phages will have the potential to help control Edw. ictaluri infections in aquaculture raised catfish without infecting beneficial bacteria that could contribute to the biological control of ESC. Interestingly, Edw. ictaluri strain RE-33 (a vaccine strain) was observed to be the most susceptible host among the isolates tested. This could be attributed to changes in the receptor site or absence of the O-side chain LPS reported in strain RE-33 (Klesius & Shoemaker 1999, Arias et al. 2003). Since the efficacy of the vaccine may be affected when both strain RE-33 and bacteriophages are used to control ESC, the vaccine strain should be applied before any bacteriophage application.

Additionally, these phages may also be used as diagnostic tools in fish disease laboratories for detection of Edw. ictaluri strains. It is reported that homogeneity exists among Edw. ictaluri strains (Plumb & Vinitnantharat, 1989, Arias et al. 2003, Panangala et al. 2006) which explains the susceptibility of all Edw. ictaluri strains (tested to date) to phage infection. No other bacterial phenotypes are known that correlate with the lower EOP for the three less phage-susceptible Edw. ictaluri strains. Variation in susceptibility among host strains may be largely due to differences in host receptor sites, modification or loss of receptor molecules, or other host resistant mechanisms such as abortive infection (Zorzopulos et al. 1979, Duckworth et al. 1981). Compared to chemotherapeutants that have a broad spectrum activity on different species (Nelson 2004), an individual phage may not effectively control aquatic pathogens, yet a “cocktail” of Edw. ictaluri specific phages may have better efficiency as a biological control strategy (O'Flynn et al. 2004, Skurnik & Strauch 2006, VernerJefferys et al. 2007). For effective biological control of ESC, additional bacteriophages would need to be identified with good infectivity for Edw. ictaluri strains 196, C91-162, and R4383; alternatively, serial passage of ΦeiAU and/or ΦeiDWF in the less-susceptible strains of Edw. ictaluri may be an effective means of enhancing the infectivity of these bacteriophages.

In vitro phage infection of Edw. ictaluri demonstrates that both phages have the potential to control ESC infections. The observations that these phages are specific to Edw. ictaluri strains, occur naturally in aquaculture ponds, and are not lysogenic encourages further work to evaluate their use as biocontrol agents for ESC. Future studies include molecular characterization of phages specific to Edw. ictaluri and evaluating the protective effects of these phages in ESC disease challenge models.

### Example 2

### Analysis of the Genomes of Edwardsiella ictaluri Bacteriophage ΦeiADWF and ΦeiAU

The double-stranded, circular genomes of Edwardsiella ictaluri bacteriophage ΦeiAU and ΦeiADWF were sequenced and are presented in single-strand, linear form as SEQ ID NO:1 and SEQ ID NO:2, respectively. The genome of ΦeiAU has 42808 nucleotides and the genome of ΦeiADWF has 42013 nucleotides. The two genomes were aligned using the Basic Local Alignment Search Tool (BLAST) available at the NBCI website (e.g., “bl2seq” as described in Tatiana A. Tatusova, Thomas L. Madden (1999), “Blast 2 sequences—a new tool for comparing protein and nucleotide sequences”, FEMS Microbiol Lett. 174:247-250)). Based on the BLAST alignment, the two genomes illustrate ˜97% sequence identity.

Open reading frames (ORFs) in the genomes of ΦeiAU and ΦeiADWF were identified, and putative functional or structural activities for the polypeptides encoded within the ORFs were identified using BLAST, Glimmer (Gene Locator and Interpolated Markov ModelER), GeneMark, and ORF Finder software. Based on the analyses, the two genomes include open reading frames (ORFS) that encode polypeptides having putative functional or structural activities as follows: SEQ ID NO:4 (HNH endonuclease [Serratia proteamaculans 568]); SEQ ID NO:5 (HNH endonuclease [Serratia proteamaculans 568]); SEQ ID NO:6 (Helicase); SEQ ID NO:7 (Helicase); SEQ ID NO:8 (Methyltransferase); SEQ ID NO:9 (N-6-adenine-methyltransferase); SEQ ID NO:10 (N-6-adenine-methyltransferase); SEQ ID NO:11 (Caudovirales tail fiber assembly protein); SEQ ID NO:12 (Caudovirales tail fiber assembly protein); SEQ ID NO:13 (Phage tail protein); SEQ ID NO:14 (Phage tail protein); SEQ ID NO:15 (Phage tail protein); SEQ ID NO:16 (Phage tail protein); SEQ ID NO:17 (Phage tail protein/phage tail assembly protein); SEQ ID NO:18 (Phage tail protein/phage tail assembly protein); SEQ ID NO:19 (Phage minor tail protein); SEQ ID NO:20 (Phage minor tail protein L); SEQ ID NO:21 (Phage minor tail protein); SEQ ID NO:22 (Phage minor tail protein); SEQ ID NO:23 (Bacteriophage tail tape measure protein); SEQ ID NO:24 (Phage protein [Proteus mirabilis HI4320]); SEQ ID NO:25 (Phage protein [Proteus mirabilis HI4320]); SEQ ID NO:26 (Protein EpSSL_gp28 [Enterobacteria phage SSL-2009a]); SEQ ID NO:27 (Major tail protein); SEQ ID NO:28 (Protein EpSSL_gp30 [Enterobacteria phage SSL-2009a]; SEQ ID NO:29 (Protein EpSSL_gp30 [Enterobacteria phage SSL-2009a]; SEQ ID NO:30 (Protein EpSSL_gp31 [Enterobacteria phage SSL-2009a]); SEQ ID NO:31 (Protein EpSSL_gp31 [Enterobacteria phage SSL-2009a]); SEQ ID NO:32 (Phage structural protein); SEQ ID NO:33 (Protein EpSSL_gp33 [Enterobacteria phage SSL-2009a]); SEQ ID NO:34 (Phage structural protein); SEQ ID NO:35 (Phage structural protein); SEQ ID NO:36 (Protein EpSSL_gp36 [Enterobacteria phage SSL-2009a]); SEQ ID NO:37 (Phage head morphogenesis protein); SEQ ID NO:38 (Phage structural protein); SEQ ID NO:39 (Phage terminase large subunit); SEQ ID NO:40 (Protein EpSSL_gp44 [Enterobacteria phage SSL-2009a]); SEQ ID NO:41 (Endolysin); SEQ ID NO:42 (Endolysin); SEQ ID NO:43 (gp119 [Lactococcus phage KSY1]); SEQ ID NO:44 (gp119 [Lactococcus phage KSY1]); SEQ ID NO:45 (Rz-like protein/phage lysis accessory protein); SEQ ID NO:46 (Phage replicative helicase/primase); SEQ ID NO:47 (Phage replicative helicase/primase); SEQ ID NO:48 (Protein EpSSL_gp14 [Enterobacteria phage SSL-2009a]); SEQ ID NO:49 (Protein EpSSL_gp14 [Enterobacteria phage SSL-2009a]); SEQ ID NO:50 (Protein EpSSL_gp11 [Enterobacteria phage SSL-2009a]); SEQ ID NO:51 (Protein EpSSL_gp11 [Enterobacteria phage SSL-2009a]); SEQ ID NO:52 (Protein BPKS7gp38 [Salmonella phage KS7]); SEQ ID NO:53 (Protein BPKS7gp38 [Salmonella phage KS7]); SEQ ID NO:54 (Protein EpSSL_gp09 [Enterobacteria phage SSL-2009a]); SEQ ID NO:55 (DNA polymerase I); SEQ ID NO:56 (Protein SPSV3_gp08 [Salmonella phage SETP3]); SEQ ID NO:57 (Holin protein); SEQ ID NO:58 (Holin protein); SEQ ID NO:59 (HNH endonuclease); SEQ ID NO:60 (HNH endonuclease); SEQ ID NO:61 (Helicase); SEQ ID NO:62; (Helicase); SEQ ID NO:63 (N-6-adenine-methyltransferase); SEQ ID NO:64 (N-6-adenine-methyltransferase); SEQ ID NO:65 (Protein T5.077 [Enterobacteria phage T5]); SEQ ID NO:66 (Protein T5.077 [Enterobacteria phage T5]); SEQ ID NO:67 (Phage tail fiber assembly protein); SEQ ID NO:68 (Phage tail protein); SEQ ID NO:69 (Phage host specificity protein); SEQ ID NO:70 (Phage host specificity protein); SEQ ID NO:71 (Phage tail protein); SEQ ID NO:72 (Phage tail protein); SEQ ID NO:73 (Phage minor tail protein); SEQ ID NO:74 (Phage minor tail protein L); SEQ ID NO:75 (Phage minor tail family protein); SEQ ID NO:76 (Phage minor tail protein precursor H); SEQ ID NO:77 (Phage minor tail protein precursor H); SEQ ID NO:78 (Phage protein [Proteus mirabilis HI4320]); SEQ ID NO:79 (Phage protein [Proteus mirabilis HI4320]); SEQ ID NO:80 (Protein EpSSL_gp28 [Enterobacteria phage SSL-2009a]); SEQ ID NO:81 (Major tail protein); SEQ ID NO:82 (Phage protein [Proteus mirabilis HI4320]); SEQ ID NO:83 (Protein EpSSL_gp31 [Enterobacteria phage SSL-2009a]; SEQ ID NO:84 (Protein EpSSL_gp31 [Enterobacteria phage SSL-2009a]); SEQ ID NO:85 (Phage structural protein); SEQ ID NO:86 (Phage structural protein); SEQ ID NO:87 (Protein EpSSL_gp33 [Enterobacteria phage SSL-2009a]); SEQ ID NO:88 (Phage structural protein); SEQ ID NO:89 (Protein EpSSL_gp36 [Enterobacteria phage SSL-2009a]); SEQ ID NO:90 (Phage head morphogenesis protein); SEQ ID NO:91 (Phage structural protein); SEQ ID NO:92 (Phage terminase large subunit); SEQ ID NO:93 (Protein EpSSL_gp44 [Enterobacteria phage SSL-2009a]); SEQ ID NO:94 (Endolysin); SEQ ID NO:95 (Endolysin); SEQ ID NO:96 (Rz-like protein/phage lysis accessory protein); SEQ ID NO:97 (Phage replicative helicase/primase); SEQ ID NO:98 (Phage replicative helicase/primase); SEQ ID NO:99 (Protein EpSSL_gp14 [Enterobacteria phage SSL-2009a]); SEQ ID NO:100 (Protein BPKS7gp38 [Salmonella phage KS7]); SEQ ID NO:101 (Protein BPKS7gp38 [Salmonella phage KS7]); SEQ ID NO:102 (Protein EpSSL_gp09 [Enterobacteria phage SSL-2009a]); SEQ ID NO:103 (DNA polymerase I); SEQ ID NO:104 (Protein SPSV3_gp08 [Salmonella phage SETP3]); SEQ ID NO:105 (Holin protein); and SEQ ID NO:106 (Holin protein).

### Example 3

### Passage of Edwardsiella ictaluri Bacteriophage ΦeiAU on Edwardsiella ictaluri Strain C91-162

As discussed in Example 1, Edwardsiella ictaluri bacteriophage ΦeiAU produced small pin-point plaques on Edwardsiella ictaluri strain C91-162 (i.e., plaques less than about 0.5 mm in size). As such, bacteriophage ΦeiAU was passaged on Edw. ictaluri strain C91-162 until an increase in plaque size was observed (i.e., until a plaque size of between about 0.5-4 mm was observed). After which, a single phage was cloned and termed “bacteriophage C91-162,” in view of its passage on the strain C91-162 and its capability to produce larger plaques than the parent bacteriophage ΦeiAU. The genome of bacteriophage C91-162 was sequenced and is presented in single strand, linear form as SEQ ID NO:3. The genome of bacteriophage C91-162 is 42923 nucleotides in length and illustrates approximately 97% sequence identity with the genome of bacteriophage ΦeiAU.

### REFERENCES

Adams, M. H. (1959) Bacteriophages. Interscience, New York, N.Y., USA.
Arias, C. R., Shoemaker, C. A., Evans, J. J., Klesius, P. H. (2003) Comparative study of Edw. ictaluri parent (EILO) and E. ictaluri rifampicin-mutant (RE-33) isolates using lipopolysaccharides, outer membrane proteins, fatty acids, Biolog, API 20E and genomic analyses. J Fish Dis 26, 415-421.
Barrow, P. A. (2001) Review The use of bacteriophages for treatment and prevention of bacterial disease in animals and animal models of human infection. J Chem Technol Biotechnol 76, 677-682.
Barrow, P. A., Soothhill, J. S. (1997) Bacteriophage therapy and prophylaxis: rediscovery and renewed assessment of potential. Trend Microbiol 5, 268-271.
Bullock, G. L., Hsu, T. C., and Shotts, E. B., Jr. (1986) Columnaris disease of fishes. U.S. Fish and Wildlife Service, Fish Disease Leaflet 72.
Dabrowska, K., Switala-Jelen, K., Opolski, A., Weber-Dabrowska, B., Gorski, A. (2005) A Review Bacteriophage penetration in vertebrates. J Appl Microbiol 98, 7-13.
DePaola, A., Peeler, J. T., Rodrick, G. E. (1995) Oxytetracycline-medicated feed on antibiotic resistance of gram-Negative bacteria in Catfish Ponds. Appl Environ Microbiol 61, 2335-2340.
Durborrow, R. M., Taylor, P. W., Crosby, M. D., Santucii, T. D. (1991) Fish mortality in the Mississippi Catfish Farming Industry in 1988: Causes and treatment. J Wildlife Dis 27, 144-147.
d'Herelle, M. D. (1926) The Bacteriophage and its behaviour. The Williams and Wilkins company, Baltimore, Md., USA.
Duckworth, D. H., Glenn, J., McCorquodale, D. J. (1981) Inhibition of bacteriophage replication by extrachromosal genetic elements. Microbial Rev 45, 52-71.
Fortier, L. C., Moineau, S. (2007) Morphological and genetic diversity of temperate phages in Clostridium difficile. Appl Environ Microbiol 73, 7358-7366.
Francis-Floyd, H., Beleau, M. H., Waterstrat, P., Bowser, P. R. (1987) Effect of temperature on clinical outcome of infection with Edw. ictaluri in channel catfish. J Am Vet Med Assoc 191, 1413-416.
Fujimura, R., Keasberg, P. (1962) The adsorption of bacteriophage {phi} X174 to its host. Biophys J 2, 433-449.
Goh, S., Riley, T. V., Chang, B. J. (2005) Isolation and characterization of temperate bacteriophages of Clostridium difficile. Appl Environ Microbiol 71, 1079-1083.
Hawke, J. P., McWhorter, A. C., Steigerwalt, C., Brenner, D. J. (1981) Edw. ictaluri sp. nov, the causative agent of enteric septicemia of catfish. Int J Syst Bact 31, 396-400.
Hawke, J. P., Durborow, R. M., Thune, R. L., Camus, A. C. (1998) Enteric Septicemia of Catfish. SRAC Publication No. 477.
Hawke J P, Khoo L H (2004) Infectious diseases. In: Biology and culture of channel catfish (ed. by C S Tucker & J A Hargreaves) Elsevier, Amsterdam, the Netherlands. pp 387-443.
Imbeault, S., Parent, S., Legace, M., Uhland, C. F., Blais, J. F. (2006) Using bacteriophages to prevent furunculosis caused by Aeromonas salmonicida in farmed brook trout. J Aquat Anim Health 18, 203-214.
Inglis, V., Roberts, R. J., Bromage, N. R. (1993) Enteric septicemia of catfish. In: Bacterial Diseases of Fish. Blackwell Science Ltd. Osney Mead, Oxford, London, p 67-79.
Johnson, M. R. (1991) Bacterial resistance to antibiotics: a growing problem in the channel catfish industry. In: Reigh RC(ed) Proceedings of Louisiana Aquaculture Conference. Louisiana State University Agricultural Center, Baton Rouge, La., p 22-23.
Klesius, P. H., Shoemaker, C. A. (1999) Development and use of modified live Edw. ictaluri vaccine against enteric septicemia of catfish. In: Schultz RD(ed), Advances in Veterinary Medicine 41, 523-537.
Li, T., Xiang, J., Liu, R., Ding, M., Shi, P., Wang, S. (1999) Studies on bacteriophage control pustule disease of abalone Haliotis discus hannai. Oceanologia et Limnologia Sinica, 30, 374-380.
Luria, S. E., Steiner, D. I. (1954) The role of calcium in the penetration of bacteriophage T5 into its host. J Bacteriol 67, 635-639.
Merril, C. R., Biswas, B., Carlton, R., Jensen, N. C., Creed, G. J., Zullo, S., Adhya, S. (1996) Long-circulating bacteriophage as antibacterial agents. Microbiol 93, 3188-3192.
Moldovan, R., Chapman-McQuiston, E., Wu, X. L. (2007) On kinetics of phage adsorption. Biophys J 93, 303-315.
Murphy, F. A., Fauquet, C. M., Bishop, D. H. L., Ghabrial, S. A., Jarvis, A. W., Martelli, G. P, Mayo, M. A. and Summers, M. D. (1995) Virus taxonomy-6^{th} report of ICTV.
Virology Division, International Union of Microbiological Societies, SpringerVerlag, New York, p 1-586.
Nakai, T., Sugimoto, R., Park, K. H., Matsuoka, S., Mori, K., Nishioka, T., Maruyama, K. (1999) Protective effects of bacteriophage on experimental Lactococcus garvieae infection in yellowtail. Dis Aquat Org 37, 33-41.
Nakai, T., Park, S. C. (2002) Bacteriophage therapy of infectious diseases in aquaculture. Res Microbiol 153, 13-18.
Nelson, D. (2004) Phage taxonomy: We agree to disagree. J Bacterial 186, 7029-7031.
O'Flynn, G., Ross, R. P., Fitzgerald, G. F., Coffey, A. (2004) Evaluation of a cocktail of three bacteriophage for biocontrol of Escherichia coli 0157: H7. Appl Environ Microbiol 70, 3417-3424.
Panangala, V. S., Shoemaker, C. A., McNulty, S. T., Arias, C. R., Klesius, P. H. (2006) Intra- and interspecific phenotypic characteristics of fish-pathogenic Edw. ictaluri and Edw. tarda. Aquaculture Res 37, 49-60.
Park, S. C., Shimarura, I., Fukunaga, M., Mori, K., Nakai, T. (2000) Isolation of bacteriophages specific to a fish pathogen, Pseudomonas plecoglossicida, as a candidate for disease control. Appl Environ Microbiol 66, 1416-1422.
Pelon, W., Luftig, R. B., Johnston, K. H. (2005) Vibrio vulnificus load reduction in Oysters after combined exposure to V. vulnificus-specific bacteriophage and an oyster extract component. J Food Prot 68, 1188-1191.
Plumb, J. A., Vinitnantharat, S. (1989) Biochemical, Biophysical, and Serological Homogeneity of Edw. ictaluri. J Aquat Anim Health 1, 51-56.
Plumb, J. A., Sheifinger, C. C., Shryock, T. R., Goldsby, T. (1995) Susceptibility of six bacterial pathogens of channel catfish to six antibiotics. J Aquat Anim Health 7, 211-217.
Plumb, J. A. (1999) Catfish bacterial diseases. In: Health maintenance and principle microbial diseases of cultured fishes. Iowa State University Press, Ames, Iowa, p 187-194.
Poranen, M. M., Ravantii, J. J., Grahn, A. M., Gupta, R., Auvinen, P., Bamford, D. H. (2006) Global changes in cellular gene expression during bacteriophage PRD1 infection. J Viral 80, 8081-8088.
Russell, W. J., Taylor, S. A., Sigel, M. M. (1976) Clearance of bacteriophage in poikilothermic vertebrates and the effect of temperature. J Reticuloendothelial Soc 19, 91.
Russell, M., Whirlow, H., Sun, T., Webster, R. E. (1988) Low-frequency infection of F-bacteria by transducing particles of filamentous bacteriophages. J Bacteriol 170, 5312-5316.
Shoemaker, C. A., Klesius. P. H., Bricker, J. M. (1999) Efficacy of a modified live Edw. ictaluri vaccine in channel catfish as young as seven days post hatch. Aquaculture 176, 189-193.
Skurnik, M., Strauch, E. (2006) Review Phage therapy: facts and fiction. Int J Med Microbiol 296, 5-14.
Su, M. T., Venkatesh., T. V., Bodmer, R. (1998) Large- and small-scale preparation of bacteriophage lysate and DNA. BioTechniques 25, 44-45.
Taddei, F., Paepe, M. (2006) Viruses' life history: Towards a mechanistic basis of a trade-off between survival and reproduction among phages. PLoS Biol 4, e273.
Tai-wu, L (2000) Studies on phage control of pustule disease in abalone Haliotis discus hannai. J Shellfish Res 19, 535.
Thune, R. L., Hawke, J. P., Johnson, M. C. (1994) Studies on vaccination of channel catfish, Ictalurus punctatus, against Edw. Ictaluri. J Appl Aquaculture 3, 11-23.
Tucker, C. S., Robinson, E. H. (1990) Channel catfish farming handbook. Van Noslrand Reinhold, New York, N.Y.
US Department of Agriculture, USDA (2003a) Part I: Reference of fingerling catfish health and production practices in the United States. National health monitoring system, 2003a, Fort Collins, Colo., USA.
US Department of Agriculture, USDA (2003b) Part II: Reference of foodsize catfish health and production practices in the United States. National health monitoring system, 2003b, Fort Collins, Colo., USA.
Verner-Jefferys, D. W., Aligoet, M., Pond, M. J., Virdee, H. K., Bagwell, N. J., Robert, E. G. (2007) Furunculosis in Atlantic salmon (Salmo salar L.) is not readily controllable by bacteriophage therapy. Aquaculture 270, 475-484.
Wagner, B. A., Wise, D. J., Khoo, L. H., Terhune, J. S. (2002) The epidemiology of bacterial diseases in food-size channel catfish. J Aquat Anim Health 14, 263-272.
Watanabe, K., Takesue, S. (1972) The requirement for Calcium in infection with Lactobacillus phage. J gen Virol 17, 19-30.
Wise D J, Johnson M J (1998) Effects of feeding frequency and Romet-medicated feed on survival, antibody response, and weight gain of fingerling channel catfish Ictalurus punctatus after natural exposure to Edw. ictaluri. J World Aquaculture Society 29: 169-175.
Wise, D. J., Terhune, J. S. (2001) The relationship between vaccine dose and efficacy in channel catfish Ictalurus punctatus vaccinated as fry with a live attenuated strain of Edw. ictaluri (RE-33) J World Aquaculture Society 32, 177-183.
Wu J, Lin H, Jan L, Hsu Y, Chang L (1981) Biological control of fish bacterial pathogen, Aeromonas hydrophila, by bacteriophage AH1. Fish Pathol 15: 271-276.
Wu, J., Chao, W. J. (1984) The epizootic of milkfish vibriosis and its biological control by bacteriophage AS10. COA Fisheries No. 10 Fish Dis Res 6, 34-46.
Zhang, Y., Arias, C. R. (2006) Identification and characterization of an intervening sequence within the 23R ribosomal RNA genes of Edw. ictaluri. Syst. Appl. Microbiol. 27, 573-580.
Zimmer, M, Scherer, S., Loessner, M. J. (2002) Genomic analysis of Clostridium perfringens bacteriophage Φ3626, which integrates into guaA and possibly affects sporulation. J Bacteriol. 184, 4359-4368.
Zorzopulos, J., Kozloff, L. M, Chapman, V., Delong, S. (1979) Bacteriophage T4D receptors and the Escherichia coli cell wall structure: Role of spherical particles and protein b of the cell wall in bacteriophage infection. J Bacteriol 137, 545-555.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member, any subgroup of members of the Markush group or other group, or the totality of members of the Markush group or other group.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. An isolated bacteriophage selected from a group consisting of ΦeiAU, ΦeiDWF, and variant bacteriophage thereof, wherein the bacteriophage has lytic activity against Edwardsiella ictaluri.

2. The isolated bacteriophage of claim 1, wherein the isolated bacteriophage is ΦeiAU.

3. The isolated bacteriophage of claim 1, wherein the isolated bacteriophage is ΦeiDWF.

4. The isolated bacteriophage of claim 1, wherein the isolated bacteriophage has a genome comprising a polynucleotide sequence of SEQ ID NO:1.

5. The isolated bacteriophage of claim 1, wherein the isolated bacteriophage has a genome comprising a polynucleotide sequence of SEQ ID NO:2.

6. The isolated bacteriophage of claim 1, wherein the isolated bacteriophage has a genome comprising a polynucleotide sequence of SEQ ID NO:3.

7. The isolated bacteriophage of claim 1, wherein the variant bacteriophage has a genome comprising a polynucleotide sequence that is a full-length variant of SEQ ID NO:1 based on degeneracy of the genetic code.

8. The isolated bacteriophage of claim 1, wherein the variant bacteriophage has a genome comprising a polynucleotide sequence that is a full-length variant of SEQ ID NO:2 based on degeneracy of the genetic code.

9. The isolated bacteriophage of claim 1, wherein the variant bacteriophage has a genome comprising a polynucleotide sequence that is a full-length variant of SEQ ID NO:3 based on degeneracy of the genetic code.

10. The isolated bacteriophage of claim 1, wherein the variant bacteriophage has a genome comprising a polynucleotide sequence that is a variant of SEQ ID NO:1 having at least 95% sequence identity to SEQ ID NO:1.

11. The isolated bacteriophage of claim 1, wherein the variant bacteriophage has a genome comprising a polynucleotide sequence that is a variant of SEQ ID NO:1 having at least 95% sequence identity to SEQ ID NO:2.

12. The isolated bacteriophage of claim 1, wherein the variant bacteriophage has a genome comprising a polynucleotide sequence that is a variant of SEQ ID NO:1 having at least 95% sequence identity to SEQ ID NO:3.

13. A method for killing Edwardsiella ictaluri bacteria comprising contacting the bacteria with a bacteriophage of claim 1.

14. The method of claim 13, wherein the bacteria are present in a pond.

15. The method of claim 14, wherein the pond comprises catfish.

16. A method for replicating the bacteriophage of claim 1, comprising infecting Edwardsiella ictaluri bacteria with the bacteriophage and incubating the infected bacteria.

17. An isolated polynucleotide comprising a polynucleotide sequence selected from one of SEQ ID NOs:1-3 or a variant polynucleotide thereof having at least 95% polynucleotide sequence identity to one of SEQ ID NOs:1-3.

18. An isolated polynucleotide encoding a polypeptide comprising an amino acid sequence of one of SEQ ID NOs:4-106 or a variant amino acid sequence thereof wherein the polypeptide has at least 95% amino acid sequence identity to one of SEQ ID NOs:4-106 and the polypeptide has a functional or structural activity selected from DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity.

19. A recombinant polynucleotide comprising a promoter sequence operably linked to the polynucleotide of claim 18.

20. An isolated cell transformed with the recombinant polynucleotide of claim 19.

21. A method of producing a polypeptide encoded by the polynucleotide of claim 18, the method comprising:
a) culturing a cell under conditions suitable for expression of the polypeptide, wherein said cell is transformed with a recombinant polynucleotide, and said recombinant polynucleotide comprises a promoter sequence operably linked to the polynucleotide of claim 18; and
b) recovering the polypeptide so expressed.

22. A vector comprising the recombinant polynucleotide of claim 19.

23. An isolated polypeptide comprising an amino acid sequence selected from one of SEQ ID NOs:4-106 or a variant polypeptide thereof having at least 95%'amino acid sequence identity to one of SEQ ID NOs:4-106, wherein the polypeptide or the variant polypeptide has a functional activity selected from DNA polymerase protein activity, Primase protein activity, Holin protein activity, Lysis protein activity, Endolysin protein activity, Terminase protein activity, Structural protein activity, Tail protein activity, DNA methylase protein activity, and Helicase protein activity.
